(19) United States
(12) Patent Application Publication
(10) Pub. No.: US 2018/0016244 A1
(43) Pub. Date: Jan. 18, 2018

(54) DEUTERIUM-SUBSTITUTED OXADIAZOLES

(71) Applicant: Auspex Pharmaceuticals, Inc., La Jolla, CA (US)

(72) Inventors: Chengzhi Zhang, San Diego, CA (US); Justin Chakma, San Diego, CA (US)

(21) Appl. No.: 15/032,881

(22) PCT Filed: Mar. 25, 2016
(86) PCT No.: PCT/US2016/024143

§ 371 Date: Apr. 28, 2016

Related U.S. Application Data
(60) Provisional application No. 62/143,489, filed on Apr. 6, 2015.

(51) Int. Cl. C07D 271/06; C07B 59/00

(52) U.S. Cl. CPC C07D 271/06; C07B 59/002; C07B 2200/05

(57) ABSTRACT

Described are deuterated modulators of S1P1 receptors, pharmaceutical compositions thereof, and methods of use thereof.

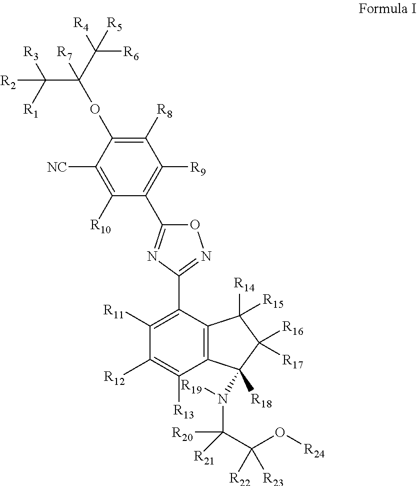

### CROSS-REFERENCE TO RELATED APPLICATIONS

This application claims the benefit of U.S. Provisional Application No. 62/143,489, filed on Apr. 6, 2015, the disclosure of which is hereby incorporated by reference in its entirety.

### TECHNICAL FIELD

Disclosed herein are new oxadiazole compounds and compositions and their application as pharmaceuticals for the treatment or prevention of disorders. Methods of modulation of sphingosine-1-phosphate subtype 1 receptor (S1P1 receptor) activity in a subject are also provided for the treatment or prevention of disorders such as multiple sclerosis, inflammatory bowel disease, transplant rejection, adult respiratory syndrome, ulcerative colitis, influenza, and Crohn's disease.

### BACKGROUND

RCP1063 (ozanimod) (5-[3-[(1S)-2,3-dihydro-1-[(2-hydroxyethyl)amino]-1H-inden-4-yl]-1,2,4-oxadiazol-5-yl]-2-(1-methylethoxy)-benzonitrile, CAS #1306760-87-1), is a S1P1 receptor modulator. RCP1063 is currently under investigation for the treatment of relapsing multiple sclerosis and inflammatory bowel disease. RCP1063 has also shown promise in the treatment of transplant rejection, adult respiratory syndrome, ulcerative colitis, influenza, and Crohn's disease. (U.S. Pat. No. 8,466,183; U.S. Pat. No. 8,481,573; WO 2011060392)

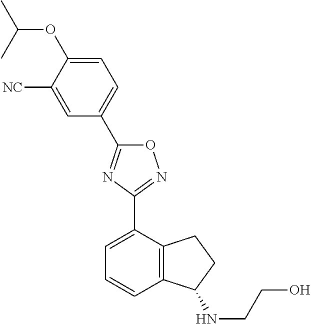 [SMILES: CC(C)Oc1ccc(-c2nc(-c3cccc4c3CC[C@@H]4NCCO)no2)cc1C#N]

### RCP1063

RCP1063 is likely subject to extensive CYP450-mediated oxidative metabolism. These, as well as other metabolic transformations, may occur in part through polymorphically-expressed enzymes, exacerbating interpatient variability. In order to overcome its short half-life, the drug likely must be taken several times per day, which increases the probability of patient incompliance and discontinuance. Additionally, some metabolites of RCP1063 may have undesirable side effects.

### Deuterium Kinetic Isotope Effect

In order to eliminate foreign substances such as therapeutic agents, the animal body expresses various enzymes, such as the cytochrome P450 enzymes (CYPs), esterases, proteases, reductases, dehydrogenases, and monoamine oxidases, to react with and convert these foreign substances to more polar intermediates or metabolites for renal excretion. Such metabolic reactions frequently involve the oxidation of a carbon-hydrogen (C—H) bond to either a carbon-oxygen (C—O) or a carbon-carbon (C—C) π-bond. The resultant metabolites may be stable or unstable under physiological conditions, and can have substantially different pharmacokinetic, pharmacodynamic, and acute and long-term toxicity profiles relative to the parent compounds. For most drugs, such oxidations are generally rapid and ultimately lead to administration of multiple or high daily doses.

The relationship between the activation energy and the rate of reaction may be quantified by the Arrhenius equation, k=Ae^{-Eact/RT}. The Arrhenius equation states that, at a given temperature, the rate of a chemical reaction depends exponentially on the activation energy (Eact).

The transition state in a reaction is a short lived state along the reaction pathway during which the original bonds have stretched to their limit. By definition, the activation energy Eact for a reaction is the energy required to reach the transition state of that reaction. Once the transition state is reached, the molecules can either revert to the original reactants, or form new bonds giving rise to reaction products. A catalyst facilitates a reaction process by lowering the activation energy leading to a transition state. Enzymes are examples of biological catalysts.

Carbon-hydrogen bond strength is directly proportional to the absolute value of the ground-state vibrational energy of the bond. This vibrational energy depends on the mass of the atoms that form the bond, and increases as the mass of one or both of the atoms making the bond increases. Since deuterium (D) has twice the mass of protium (¹H), a C-D bond is stronger than the corresponding C-¹H bond. If a C-¹H bond is broken during a rate-determining step in a chemical reaction (i.e. the step with the highest transition state energy), then substituting a deuterium for that protium will cause a decrease in the reaction rate. This phenomenon is known as the Deuterium Kinetic Isotope Effect (DKIE). The magnitude of the DKIE can be expressed as the ratio between the rates of a given reaction in which a C-¹H bond is broken, and the same reaction where deuterium is substituted for protium. The DKIE can range from about 1 (no isotope effect) to very large numbers, such as 50 or more. Substitution of tritium for hydrogen results in yet a stronger bond than deuterium and gives numerically larger isotope effects

Deuterium (²H or D) is a stable and non-radioactive isotope of hydrogen which has approximately twice the mass of protium (¹H), the most common isotope of hydrogen. Deuterium oxide (D₂O or “heavy water”) looks and tastes like H₂O, but has different physical properties.

When pure D₂O is given to rodents, it is readily absorbed. The quantity of deuterium required to induce toxicity is extremely high. When about 0-15% of the body water has been replaced by D₂O, animals are healthy but are unable to gain weight as fast as the control (untreated) group. When about 15-20% of the body water has been replaced with D₂O, the animals become excitable. When about 20-25% of the body water has been replaced with D₂O, the animals become so excitable that they go into frequent convulsions when stimulated. Skin lesions, ulcers on the paws and muzzles, and necrosis of the tails appear. The animals also become very aggressive. When about 30% of the body water has been replaced with D₂O, the animals refuse to eat and become comatose. Their body weight drops sharply and their metabolic rates drop far below normal, with death occurring at about 30 to about 35% replacement with D₂O. The effects are reversible unless more than thirty percent of the previous body weight has been lost due to D₂O. Studies have also shown that the use of D₂O can delay the growth of cancer cells and enhance the cytotoxicity of certain antineoplastic agents.

Deuteration of pharmaceuticals to improve pharmacokinetics (PK), pharmacodynamics (PD), and toxicity profiles has been demonstrated previously with some classes of drugs. For example, the DKIE was used to decrease the hepatotoxicity of halothane, presumably by limiting the production of reactive species such as trifluoroacetyl chloride. However, this method may not be applicable to all drug classes. For example, deuterium incorporation can lead to metabolic switching. Metabolic switching occurs when xenogens, sequestered by Phase I enzymes, bind transiently and re-bind in a variety of conformations prior to the chemical reaction (e.g., oxidation). Metabolic switching is enabled by the relatively vast size of binding pockets in many Phase I enzymes and the promiscuous nature of many metabolic reactions. Metabolic switching can lead to different proportions of known metabolites as well as altogether new metabolites. This new metabolic profile may impart more or less toxicity. Such pitfalls are non-obvious and are not predictable a priori for any drug class.

RCP1063 is a S1P1 receptor modulator. The carbon-hydrogen bonds of RCP1063 contain a naturally occurring distribution of hydrogen isotopes, namely ¹H or protium (about 99.9844%), ²H or deuterium (about 0.0156%), and ³H or tritium (in the range between about 0.5 and 67 tritium atoms per 10¹⁸ protium atoms). Increased levels of deuterium incorporation may produce a detectable Deuterium Kinetic Isotope Effect (DKIE) that could affect the pharmacokinetic, pharmacologic and/or toxicologic profiles of such RCP1063 in comparison with the compound having naturally occurring levels of deuterium.

Based on discoveries made in our laboratory, as well as considering the literature, RCP1063 is likely metabolized in humans at the hydroxyethyl group, the isopropyl group, and the indenyl methylene and N-methine groups. The current approach has the potential to prevent metabolism at these sites. Other sites on the molecule may also undergo transformations leading to metabolites with as-yet-unknown pharmacology/toxicology. Limiting the production of these metabolites has the potential to decrease the danger of the administration of such drugs and may even allow increased dosage and/or increased efficacy. All of these transformations can occur through polymorphically-expressed enzymes, exacerbating interpatient variability. Further, some disorders are best treated when the subject is medicated around the clock or for an extended period of time. For all of the foregoing reasons, a medicine with a longer half-life may result in greater efficacy and cost savings. Various deuteration patterns can be used to (a) reduce or eliminate unwanted metabolites, (b) increase the half-life of the parent drug, (c) decrease the number of doses needed to achieve a desired effect, (d) decrease the amount of a dose needed to achieve a desired effect, (e) increase the formation of active metabolites, if any are formed, (0 decrease the production of deleterious metabolites in specific tissues, and/or (g) create a more effective drug and/or a safer drug for polypharmacy, whether the polypharmacy be intentional or not. The deuteration approach has the strong potential to slow the metabolism of RCP1063 and attenuate interpatient variability.

### SUMMARY

Novel compounds and pharmaceutical compositions, certain of which have been found to modulate S1P1 receptor have been discovered, together with methods of synthesizing and using the compounds, including methods for the treatment or prevention of S1P1 receptor-mediated disorders in a patient by administering the compounds.

### DETAILED DESCRIPTION

In certain embodiments of the present invention, compounds have structural Formula I:

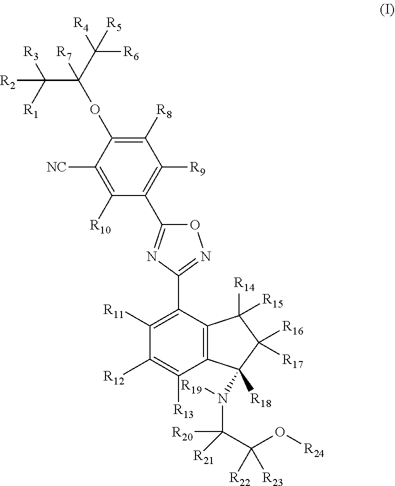

or a salt thereof, wherein:

R₁-R₂₄ are independently selected from the group consisting of hydrogen and deuterium; and

at least one of R₁-R₂₄ is deuterium or contains deuterium.

In certain embodiments, R₇ is deuterium.

In certain embodiments, R₁-R₆ are deuterium.

In certain embodiments, R₁-R₇ are deuterium.

In certain embodiments, R₁₈ is deuterium.

In certain embodiments, R₇ and R₁₈ are deuterium.

In certain embodiments, R₁-R₆ and R₁₈ are deuterium.

In certain embodiments, R₁-R₇ and R₁₈ are deuterium.

In certain embodiments, R₂₀-R₂₁ are deuterium.

In certain embodiments, R₇ and R₂₀-R₂₁ are deuterium.

In certain embodiments, R₁-R₆ and R₂₀-R₂₁ are deuterium.

In certain embodiments, R₁-R₇ and R₂₀-R₂₁ are deuterium.

In certain embodiments, R₁₈ and R₂₀-R₂₁ are deuterium.

In certain embodiments, R₇, R₁₈, and R₂₀-R₂₁ are deuterium.

In certain embodiments, R₁-R₆, R₁₈, and R₂₀-R₂₁ are deuterium.

In certain embodiments, R₁-R₇, R₁₈, and R₂₀-R₂₁ are deuterium.

In certain embodiments, R₂₂-R₂₃ are deuterium.

In certain embodiments, R₇ and R₂₂-R₂₃ are deuterium.

In certain embodiments, R₁-R₆ and R₂₂-R₂₃ are deuterium.

In certain embodiments, R₁-R₇ and R₂₂-R₂₃ are deuterium.

In certain embodiments, R₁₈ and R₂₂-R₂₃ are deuterium.

In certain embodiments, R₇, R₁₈, and R₂₂-R₂₃ are deuterium.

In certain embodiments, R₁-R₆, R₁₈, and R₂₂-R₂₃ are deuterium.

In certain embodiments, R₁-R₇, R₁₈, and R₂₂-R₂₃ are deuterium.

In certain embodiments, R₂₀-R₂₃ are deuterium.

In certain embodiments, R₇ and R₂₀-R₂₃ are deuterium.

In certain embodiments, R₁-R₆ and R₂₀-R₂₃ are deuterium.

In certain embodiments, R₁-R₇ and R₂₀-R₂₃ are deuterium.

In certain embodiments, R₁₈ and R₂₀-R₂₃ are deuterium.

In certain embodiments, R₇ and R₁₈ are deuterium.

In certain embodiments, R₁-R₆, R₁₈, and R₂₀-R₂₃ are deuterium.

In certain embodiments, R₁-R₇, R₁₈, and R₂₀-R₂₃ are deuterium.

Also provided herein are embodiments according to each of the embodiments above, wherein R₁₄-R₁₅ are deuterium.

Also provided herein are embodiments according to each of the embodiments above, wherein R₁₆-R₁₇ are deuterium.

Also provided herein are embodiments according to each of the embodiments above, wherein R₁₄-R₁₇ are deuterium.

Also provided herein are embodiments according to each of the embodiments above, wherein R₁₉ is hydrogen.

Also provided herein are embodiments according to each of the embodiments above, wherein R₂₄ is hydrogen.

Also provided herein are embodiments according to each of the embodiments above, wherein every other substituent among R₁-R₂₄ not specified as deuterium is hydrogen.

In certain embodiments are provided compounds as disclosed herein, wherein at least one of R₁-R₂₄ independently has deuterium enrichment of no less than about 1%. In certain embodiments are provided compounds as disclosed herein, wherein at least one of R₁-R₂₄ independently has deuterium enrichment of no less than about 10%. In certain embodiments are provided compounds as disclosed herein, wherein at least one of R₁-R₂₄ independently has deuterium enrichment of no less than about 50%. In certain embodiments are provided compounds as disclosed herein, wherein at least one of R₁-R₂₄ independently has deuterium enrichment of no less than about 90%. In certain embodiments are provided compounds as disclosed herein, wherein at least one of R₁-R₂₄ independently has deuterium enrichment of no less than about 95%. In certain embodiments are provided compounds as disclosed herein, wherein at least one of R₁-R₂₄ independently has deuterium enrichment of no less than about 98%.

Certain compounds disclosed herein may possess useful S1P1 receptor modulating activity, and may be used in the treatment or prophylaxis of a disorder in which S1P1 receptors play an active role. Thus, certain embodiments also provide pharmaceutical compositions comprising one or more compounds disclosed herein together with a pharmaceutically acceptable carrier, as well as methods of making and using the compounds and compositions. Certain embodiments provide methods for modulating S1P1 receptor. Other embodiments provide methods for treating a S1P1 receptor-mediated disorder in a patient in need of such treatment, comprising administering to said patient a therapeutically effective amount of a compound or composition according to the present invention. Also provided is the use of certain compounds disclosed herein for use in the manufacture of a medicament for the prevention or treatment of a disorder ameliorated by the modulation of S1P1 receptors.

The compounds as disclosed herein may also contain less prevalent isotopes for other elements, including, but not limited to, ¹³C or ¹⁴C for carbon, ³³S, ³⁴S, or ³⁶S for sulfur, ¹⁵N for nitrogen, and ¹⁷O or ¹⁸O for oxygen.

In certain embodiments, the compound disclosed herein may expose a patient to a maximum of about 0.000005% D₂O or about 0.00001% DHO, assuming that all of the C-D bonds in the compound as disclosed herein are metabolized and released as D₂O or DHO. In certain embodiments, the levels of D₂O shown to cause toxicity in animals is much greater than even the maximum limit of exposure caused by administration of the deuterium enriched compound as disclosed herein. Thus, in certain embodiments, the deuterium-enriched compound disclosed herein should not cause any additional toxicity due to the formation of D₂O or DHO upon drug metabolism.

In certain embodiments are provided compounds as disclosed herein, wherein each position represented as D has deuterium enrichment of no less than about 1%. In certain embodiments are provided compounds as disclosed herein, wherein each position represented as D has deuterium enrichment of no less than about 10%. In certain embodiments are provided compounds as disclosed herein, wherein each position represented as D has deuterium enrichment of no less than about 50%. In certain embodiments are provided compounds as disclosed herein, wherein each position represented as D has deuterium enrichment of no less than about 90%. In certain embodiments are provided compounds as disclosed herein, wherein each position represented as D has deuterium enrichment of no less than about 95%. In certain embodiments are provided compounds as disclosed herein, wherein each position represented as D has deuterium enrichment of no less than about 98%.

In certain embodiments, the deuterated compounds disclosed herein maintain the beneficial aspects of the corresponding non-isotopically enriched molecules while substantially increasing the maximum tolerated dose, decreasing toxicity, increasing the half-life (T_{1/2}), lowering the maximum plasma concentration (Cₘₐₓ) of the minimum efficacious dose (MED), lowering the efficacious dose and thus decreasing the non-mechanism-related toxicity, and/or lowering the probability of drug-drug interactions.

All publications and references cited herein are expressly incorporated herein by reference in their entirety. However, with respect to any similar or identical terms found in both the incorporated publications or references and those explicitly put forth or defined in this document, then those terms definitions or meanings explicitly put forth in this document shall control in all respects.

As used herein, the terms below have the meanings indicated.

The singular forms “a,” “an,” and “the” may refer to plural articles unless specifically stated otherwise.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

When ranges of values are disclosed, and the notation “from n₁ . . . to n₂” or “n₁-n₂” is used, where n₁ and n₂ are the numbers, then unless otherwise specified, this notation is intended to include the numbers themselves and the range between them. This range may be integral or continuous between and including the end values.

The term “deuterium enrichment” refers to the percentage of incorporation of deuterium at a given position in a molecule in the place of hydrogen. For example, deuterium enrichment of 1% at a given position means that 1% of molecules in a given sample contain deuterium at the specified position. Because the naturally occurring distribution of deuterium is about 0.0156%, deuterium enrichment at any position in a compound synthesized using non-enriched starting materials is about 0.0156%. The deuterium enrichment can be determined using conventional analytical methods known to one of ordinary skill in the art, including mass spectrometry and nuclear magnetic resonance spectroscopy.

The term “is/are deuterium,” when used to describe a given position in a molecule such as R₁-R₂₄ or the symbol “D”, when used to represent a given position in a drawing of a molecular structure, means that the specified position is enriched with deuterium above the naturally occurring distribution of deuterium. In one embodiment deuterium enrichment is no less than about 1%, in another no less than about 5%, in another no less than about 10%, in another no less than about 20%, in another no less than about 50%, in another no less than about 70%, in another no less than about 80%, in another no less than about 90%, or in another no less than about 98% of deuterium at the specified position.

The term “isotopic enrichment” refers to the percentage of incorporation of a less prevalent isotope of an element at a given position in a molecule in the place of the more prevalent isotope of the element.

The term “non-isotopically enriched” refers to a molecule in which the percentages of the various isotopes are substantially the same as the naturally occurring percentages.

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disorder” as used herein is intended to be generally synonymous, and is used interchangeably with, the terms “disease” and “condition” (as in medical condition), in that all reflect an abnormal condition of the human or animal body or of one of its parts that impairs normal functioning, is typically manifested by distinguishing signs and symptoms.

The terms “treat,” “treating,” and “treatment” are meant to include alleviating or abrogating a disorder or one or more of the symptoms associated with a disorder; or alleviating or eradicating the cause(s) of the disorder itself.

The terms “prevent,” “preventing,” and “prevention” refer to a method of delaying or precluding the onset of a disorder; and/or its attendant symptoms, barring a subject from acquiring a disorder or reducing a subject's risk of acquiring a disorder.

The term “therapeutically effective amount” refers to the amount of a compound that, when administered, is sufficient to prevent development of, or alleviate to some extent, one or more of the symptoms of the disorder being treated. The term “therapeutically effective amount” also refers to the amount of a compound that is sufficient to elicit the biological or medical response of a cell, tissue, system, animal, or human that is being sought by a researcher, veterinarian, medical doctor, or clinician.

The term “subject” refers to an animal, including, but not limited to, a primate (e.g., human, monkey, chimpanzee, gorilla, and the like), rodents (e.g., rats, mice, gerbils, hamsters, ferrets, and the like), lagomorphs, swine (e.g., pig, miniature pig), equine, canine, feline, and the like. The terms “subject” and “patient” are used interchangeably herein in reference, for example, to a mammalian subject, such as a human patient.

The term “combination therapy” means the administration of two or more therapeutic agents to treat (or prevent) a therapeutic disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment (or prevention) regimen will provide beneficial effects of the drug combination in treating the disorders described herein.

The term “sphingosine-1-phosphate subtype 1 receptor” or “S1P1 receptor” refers to a G-protein coupled receptor (GPCR) and is a member of the endothelial cell differentiation gene (EDG) receptor family. Endogenous ligands for EDG receptors include lysophospholipids, such as sphingosine-1-phosphate (S1P). Like all GPCRs, ligation of the receptor propagates second messenger signals via activation of G-proteins (alpha, beta and gamma). Agonism of the S1P1 receptor perturbs lymphocyte trafficking, sequestering them in lymph nodes and other secondary lymphoid tissue. This leads to rapid and reversible lymphopenia, and is probably due to receptor ligation on both lymphatic endothelial cells and lymphocytes themselves (Rosen et al, Immunol. Rev., 195:160-177, 2003). A clinically valuable consequence of lymphocyte sequestration is exclusion of them from sights of inflammation and/or autoimmune reactivity in peripheral tissues. Agonism of S 1P1 has also been reported to promote survival of oligodendrocyte progenitors (Miron et al, Ann. Neurol., 63:61-71, 2008). This activity, in conjunction with lymphocyte sequestration would be useful in treating inflammatory and autoimmune conditions of the central nervous system.

The term “S1P1 receptor-mediated disorder,” refers to a disorder that is characterized by abnormal S1P1 receptor activity or S1P1 receptor activity that, when modulated, leads to the amelioration of other abnormal biological processes. A S1P1 receptor-mediated disorder may be completely or partially mediated by modulating S1P1 receptors. In particular, a S1P1 receptor-mediated disorder is one in which modulation of S1P1 receptors results in some effect on the underlying disorder e.g., administration of a S1P1 receptor modulator results in some improvement in at least some of the patients being treated.

A modulator may activate the activity of a S1P1 receptor, may activate or inhibit the activity of a S1P1 receptor depending on the concentration of the compound exposed to the S1P1 receptor, or may inhibit the activity of a S1P1 receptor. Such activation or inhibition may be contingent on the occurrence of a specific event, such as activation of a signal transduction pathway, and/or may be manifest only in particular cell types. The term “S1P1 receptor modulator” or “modulation of S1P1 receptors” also refers to altering the function of an S1P1 receptor by increasing or decreasing the probability that a complex forms between a S1P1 receptor and a natural binding partner. A S1P1 receptor modulator may increase the probability that such a complex forms between the S1P1 receptor and the natural binding partner, may increase or decrease the probability that a complex forms between the S1P1 receptor and the natural binding partner depending on the concentration of the compound exposed to the S1P1 receptor, and or may decrease the probability that a complex forms between the S1P1 receptor and the natural binding partner. In some embodiments, modulation of the S1P1 receptor may be assessed using the procedures described in U.S. Pat. No. 8,466,183, U.S. Pat. No. 8,481,573, and WO 2011060392, the disclosures of which are incorporated herein by reference in their entireties.

The term “therapeutically acceptable” refers to those compounds (or salts, prodrugs, tautomers, zwitterionic forms, etc.) which are suitable for use in contact with the tissues of patients without excessive toxicity, irritation, allergic response, immunogenicity, are commensurate with a reasonable benefit/risk ratio, and are effective for their intended use.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenicity, or other problems or complications, commensurate with a reasonable benefit/risk ratio.

The terms “active ingredient,” “active compound,” and “active substance” refer to a compound, which is administered, alone or in combination with one or more pharmaceutically acceptable excipients or carriers, to a subject for treating, preventing, or ameliorating one or more symptoms of a disorder.

The terms “drug,” “therapeutic agent,” and “chemotherapeutic agent” refer to a compound, or a pharmaceutical composition thereof, which is administered to a subject for treating, preventing, or ameliorating one or more symptoms of a disorder.

The term “release controlling excipient” refers to an excipient whose primary function is to modify the duration or place of release of the active substance from a dosage form as compared with a conventional immediate release dosage form.

The term “nonrelease controlling excipient” refers to an excipient whose primary function do not include modifying the duration or place of release of the active substance from a dosage form as compared with a conventional immediate release dosage form.

The term “prodrug” refers to a compound functional derivative of the compound as disclosed herein and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis.

The compounds disclosed herein can exist as therapeutically acceptable salts. The term “therapeutically acceptable salt,” as used herein, represents salts or zwitterionic forms of the compounds disclosed herein which are therapeutically acceptable as defined herein. The salts can be prepared during the final isolation and purification of the compounds or separately by reacting the appropriate compound with a suitable acid or base. Therapeutically acceptable salts include acid and basic addition salts.

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxo-glutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

While it may be possible for the compounds of the subject invention to be administered as the raw chemical, it is also possible to present them as a pharmaceutical composition. Accordingly, provided herein are pharmaceutical compositions which comprise one or more of certain compounds disclosed herein, or one or more pharmaceutically acceptable salts, prodrugs, or solvates thereof, together with one or more pharmaceutically acceptable carriers thereof and optionally one or more other therapeutic ingredients. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes. The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art.

The compositions include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The compositions may conveniently be presented in unit dosage form and may be prepared by any of the methods well known in the art of pharmacy. Typically, these methods include the step of bringing into association a compound of the subject invention or a pharmaceutically salt, prodrug, or solvate thereof (“active ingredient”) with the carrier which constitutes one or more accessory ingredients. In general, the compositions are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose.

For administration by inhalation, compounds may be delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the disorder being treated. Also, the route of administration may vary depending on the disorder and its severity.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disorder.

In the case wherein the patient's status does improve, upon the doctor's discretion the administration of the compounds may be given continuously or temporarily suspended for a certain length of time (i.e., a “drug holiday”).

Once improvement of the patient's conditions has occurred, a maintenance dose is administered if necessary. Subsequently, the dosage or the frequency of administration, or both, can be reduced, as a function of the symptoms, to a level at which the improved disorder is retained. Patients can, however, require intermittent treatment (i.e., administration) on a long-term basis upon any recurrence of symptoms.

Disclosed herein are methods of treating a S1P1 receptor-mediated disorder comprising administering to a subject having or suspected to have such a disorder, a therapeutically effective amount of a compound as disclosed herein or a pharmaceutically acceptable salt, solvate, or prodrug thereof.

S1P1 receptor-mediated disorders, include, but are not limited to, multiple sclerosis, inflammatory bowel disease, transplant rejection, adult respiratory syndrome, ulcerative colitis, influenza, and Crohn's disease, and/or any disorder which can lessened, alleviated, or prevented by administering a S1P1 receptor modulator.

In certain embodiments, a method of treating a S1P1 receptor-mediated disorder comprises administering to the subject a therapeutically effective amount of a compound of as disclosed herein, or a pharmaceutically acceptable salt, solvate, or prodrug thereof, so as to affect: (1) decreased inter-individual variation in plasma levels of the compound or a metabolite thereof; (2) increased average plasma levels of the compound or decreased average plasma levels of at least one metabolite of the compound per dosage unit; (3) decreased inhibition of, and/or metabolism by at least one cytochrome P450 or monoamine oxidase isoform in the subject; (4) decreased metabolism via at least one polymorphically-expressed cytochrome P450 isoform in the subject; (5) at least one statistically-significantly improved disorder-control and/or disorder-eradication endpoint; (6) an improved clinical effect during the treatment of the disorder, (7) prevention of recurrence, or delay of decline or appearance, of abnormal alimentary or hepatic parameters as the primary clinical benefit, or (8) reduction or elimination of deleterious changes in any diagnostic hepatobiliary function endpoints, as compared to the corresponding non-isotopically enriched compound.

In certain embodiments, inter-individual variation in plasma levels of the compounds as disclosed herein, or metabolites thereof, is decreased; average plasma levels of the compound as disclosed herein are increased; average plasma levels of a metabolite of the compound as disclosed herein are decreased; inhibition of a cytochrome P450 or monoamine oxidase isoform by a compound as disclosed herein is decreased; or metabolism of the compound as disclosed herein by at least one polymorphically-expressed cytochrome P450 isoform is decreased; by greater than about 5%, greater than about 10%, greater than about 20%, greater than about 30%, greater than about 40%, or by greater than about 50% as compared to the corresponding non-isotopically enriched compound.

Plasma levels of the compound as disclosed herein, or metabolites thereof, may be measured using the methods described the art.

Examples of cytochrome P450 isoforms in a mammalian subject include, but are not limited to, CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R₁, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, and CYP51.

Examples of monoamine oxidase isoforms in a mammalian subject include, but are not limited to, MAO_{A}, and MAO_{B}.

The inhibition of the cytochrome P450 isoform is measured by the method of Ko et al. (British Journal of Clinical Pharmacology, 2000, 49, 343-351). The inhibition of the MAO_{A} isoform is measured by the method of Weyler et al. (J. Biol Chem. 1985, 260, 13199-13207). The inhibition of the MAO_{B} isoform is measured by the method of Uebelhack et al. (Pharmacopsychiatry, 1998, 31, 187-192).

Examples of polymorphically-expressed cytochrome P450 isoforms in a mammalian subject include, but are not limited to, CYP2C8, CYP2C9, CYP2C19, and CYP2D6.

The metabolic activities of liver microsomes, cytochrome P450 isoforms, and monoamine oxidase isoforms are measured by the methods described herein.

Examples of diagnostic hepatobiliary function endpoints include, but are not limited to, alanine aminotransferase (“ALT”), serum glutamic-pyruvic transaminase (“SGPT”), aspartate aminotransferase (“AST” or “SGOT”), ALT/AST ratios, serum aldolase, alkaline phosphatase (“ALP”), ammonia levels, bilirubin, gamma-glutamyl transpeptidase (“GGTP,” “γ-GTP,” or “GGT”), leucine aminopeptidase (“LAP”), liver biopsy, liver ultrasonography, liver nuclear scan, 5′-nucleotidase, and blood protein. Hepatobiliary endpoints are compared to the stated normal levels as given in “Diagnostic and Laboratory Test Reference”, 4^{th} edition, Mosby, 1999. These assays are run by accredited laboratories according to standard protocol.

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

### Combination Therapy

The compounds disclosed herein may also be combined or used in combination with other agents useful in the treatment or prevention of S1P1 receptor-mediated disorders. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced).

Such other agents, adjuvants, or drugs, may be administered, by a route and in an amount commonly used therefor, simultaneously or sequentially with a compound as disclosed herein. When a compound as disclosed herein is used contemporaneously with one or more other drugs, a pharmaceutical composition containing such other drugs in addition to the compound disclosed herein may be utilized, but is not required.

In certain embodiments, the compounds disclosed herein can be combined with one or more H+, K+ ATPase inhibitors, alimentary motility modulator, non-steroidal anti-inflammatory agents, anilide analgesics, anti-rheumatic agents, glucocorticoids, and immunosuppressants.

In certain embodiments, the compounds disclosed herein can be combined with one or more H+, K+ ATPase inhibitors, including, but not limited to, esomeprazole, lansoprazole, omeprazole, pantoprazole, rabeprazole, and tenatoprazole.

In certain embodiments, the compounds disclosed herein can be combined with one or more alimentary motility modulators, including, but not limited to, solabegron, tegaserod, alosetron, cilansetron, domperidone, metoclopramide, itopride, cisapride, renzapride, zacopride, octreotide, naloxone, erythromycin, and bethanechol.

In certain embodiments, the compounds disclosed herein can be combined with one or more non-steroidal anti-inflammatory agents, including, but not limited to, aceclofenac, acemetacin, amoxiprin, aspirin, azapropazone, benorilate, bromfenac, carprofen, celecoxib, choline magnesium salicylate, diclofenac, diflunisal, etodolac, etoracoxib, faislamine, fenbuten, fenoprofen, flurbiprofen, ibuprofen, indometacin, ketoprofen, ketorolac, lornoxicam, loxoprofen, lumiracoxib, meloxicam, meclofenamic acid, mefenamic acid, meloxicam, metamizole, methyl salicylate, magnesium salicylate, nabumetone, naproxen, nimesulide, oxyphenbutazone, parecoxib, phenylbutazone, piroxicam, salicyl salicylate, sulindac, sulfinprazone, suprofen, tenoxicam, tiaprofenic acid, and tolmetin.

In certain embodiments, the compounds disclosed herein can be combined with one or more anilide analgesics, including, but not limited to, acetaminophen and phenacetin.

In certain embodiments, the compounds disclosed herein can be combined with one or more disease-modifying anti-rheumatic agents, including, but not limited to, azathioprine, cyclosporine A, D-penicillamine, gold salts, hydroxychloroquine, leflunomide, methotrexate, minocycline, sulfasalazine, cyclophosphamide, etanercept, infliximab, adalimumab, anakinra, rituximab, and abatacept.

In certain embodiments, the compounds disclosed herein can be combined with one or more glucocorticoids, including, but not limited to, beclometasone, budesonide, flunisolide, betamethasone, fluticasone, triamcinolone, mometasone, ciclesonide, hydrocortisone, cortisone acetate, prednisone, prednisolone, methylprednisolone, and dexamethasone.

In certain embodiments, the compounds disclosed herein can be combined with one or more immunosuppressants, including, but not limited to, fingolimod, cyclosporine A, Azathioprine, dexamethasone, tacrolimus, sirolimus, pimecrolimus, mycophenolate salts, everolimus, basiliximab, daclizumab, anti-thymocyte globulin, anti-lymphocyte globulin, and CTLA4IgG.

The compounds disclosed herein can also be administered in combination with other classes of compounds, including, but not limited to, norepinephrine reuptake inhibitors (NRIs) such as atomoxetine; dopamine reuptake inhibitors (DARIs), such as methylphenidate; serotonin-norepinephrine reuptake inhibitors (SNRIs), such as milnacipran; sedatives, such as diazepham; norepinephrine-dopamine reuptake inhibitor (NDRIs), such as bupropion; serotonin-norepinephrine-dopamine-reuptake-inhibitors (SNDRIs), such as venlafaxine; monoamine oxidase inhibitors, such as selegiline; hypothalamic phospholipids; endothelin converting enzyme (ECE) inhibitors, such as phosphoramidon; opioids, such as tramadol; thromboxane receptor antagonists, such as ifetroban; potassium channel openers; thrombin inhibitors, such as hirudin; hypothalamic phospholipids; growth factor inhibitors, such as modulators of PDGF activity; platelet activating factor (PAF) antagonists; anti-platelet agents, such as GPIIb/IIIa blockers (e.g., abdximab, eptifibatide, and tirofiban), P2Y(AC) antagonists (e.g., clopidogrel, ticlopidine and CS-747), and aspirin; anticoagulants, such as warfarin; low molecular weight heparins, such as enoxaparin; Factor VIIa Inhibitors and Factor Xa Inhibitors; renin inhibitors; neutral endopeptidase (NEP) inhibitors; vasopepsidase inhibitors (dual NEP-ACE inhibitors), such as omapatrilat and gemopatrilat; HMG CoA reductase inhibitors, such as pravastatin, lovastatin, atorvastatin, simvastatin, NK-104 (a.k.a. itavastatin, nisvastatin, or nisbastatin), and ZD-4522 (also known as rosuvastatin, or atavastatin or visastatin); squalene synthetase inhibitors; fibrates; bile acid sequestrants, such as questran; niacin; anti-atherosclerotic agents, such as ACAT inhibitors; MTP Inhibitors; calcium channel blockers, such as amlodipine besylate; potassium channel activators; alpha-muscarinic agents; beta-muscarinic agents, such as carvedilol and metoprolol; antiarrhythmic agents; diuretics, such as chlorothlazide, hydrochiorothiazide, flumethiazide, hydroflumethiazide, bendroflumethiazide, methylchlorothiazide, trichioromethiazide, polythiazide, benzothlazide, ethacrynic acid, tricrynafen, chlorthalidone, furosenilde, musolimine, bumetanide, triamterene, amiloride, and spironolactone; thrombolytic agents, such as tissue plasminogen activator (tPA), recombinant tPA, streptokinase, urokinase, prourokinase, and anisoylated plasminogen streptokinase activator complex (APSAC); anti-diabetic agents, such as biguanides (e.g. metformin), glucosidase inhibitors (e.g., acarbose), insulins, meglitinides (e.g., repaglinide), sulfonylureas (e.g., glimepiride, glyburide, and glipizide), thiozolidinediones (e.g. troglitazone, rosiglitazone and pioglitazone), and PPAR-gamma agonists; mineralocorticoid receptor antagonists, such as spironolactone and eplerenone; growth hormone secretagogues; aP2 inhibitors; phosphodiesterase inhibitors, such as PDE III inhibitors (e.g., cilostazol) and PDE V inhibitors (e.g., sildenafil, tadalafil, vardenafil); protein tyrosine kinase inhibitors; antiinflammatories; antiproliferatives, such as methotrexate, FK506 (tacrolimus, Prograf), mycophenolate mofetil; chemotherapeutic agents; immunosuppressants; anticancer agents and cytotoxic agents (e.g., alkylating agents, such as nitrogen mustards, alkyl sulfonates, nitrosoureas, ethylenimines, and triazenes); antimetabolites, such as folate antagonists, purine analogues, and pyrridine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids (e.g., cortisone), estrogens/antiestrogens, androgens/antiandrogens, progestins, and luteinizing hormone-releasing hormone anatagonists, and octreotide acetate; microtubule-disruptor agents, such as ecteinascidins; microtubule-stablizing agents, such as pacitaxel, docetaxel, and epothilones A-F; plant-derived products, such as vinca alkaloids, epipodophyllotoxins, and taxanes; and topoisomerase inhibitors; prenyl-protein transferase inhibitors; and cyclosporins; steroids, such as prednisone and dexamethasone; cytotoxic drugs, such as azathiprine and cyclophosphamide; TNF-alpha inhibitors, such as tenidap; anti-TNF antibodies or soluble TNF receptor, such as etanercept, rapamycin, and leflunimide; and cyclooxygenase-2 (COX-2) inhibitors, such as celecoxib and rofecoxib; and miscellaneous agents such as, hydroxyurea, procarbazine, mitotane, hexamethylmelamine, gold compounds, platinum coordination complexes, such as cisplatin, satraplatin, and carboplatin.

Thus, in another aspect, certain embodiments provide methods for treating or preventing S1P1 receptor-mediated disorders in a human or animal subject in need thereof comprising administering to said subject an amount of a compound disclosed herein effective to reduce or prevent said disorder in the subject, in combination with at least one additional agent for the treatment or prevention of said disorder that is known in the art. In a related aspect, certain embodiments provide therapeutic compositions comprising at least one compound disclosed herein in combination with one or more additional agents for the treatment or prevention of S1P1 receptor-mediated disorders.

### General Synthetic Methods for Preparing Compounds

Isotopic hydrogen can be introduced into a compound as disclosed herein by synthetic techniques that employ deuterated reagents, whereby incorporation rates are predetermined; and/or by exchange techniques, wherein incorporation rates are determined by equilibrium conditions, and may be highly variable depending on the reaction conditions. Synthetic techniques, where tritium or deuterium is directly and specifically inserted by tritiated or deuterated reagents of known isotopic content, may yield high tritium or deuterium abundance, but can be limited by the chemistry required. Exchange techniques, on the other hand, may yield lower tritium or deuterium incorporation, often with the isotope being distributed over many sites on the molecule.

The compounds as disclosed herein can be prepared by methods known to one of skill in the art and routine modifications thereof, and/or following procedures similar to those described in the Example section herein and routine modifications thereof, and/or procedures found in U.S. Pat. No. 8,466,183, U.S. Pat. No. 8,481,573, and WO 2011060392, which are hereby incorporated in their entirety, and references cited therein and routine modifications thereof. Compounds as disclosed herein can also be prepared as shown in any of the following schemes and routine modifications thereof.

The following schemes can be used to practice the present invention. Any position shown as hydrogen may optionally be replaced with deuterium.

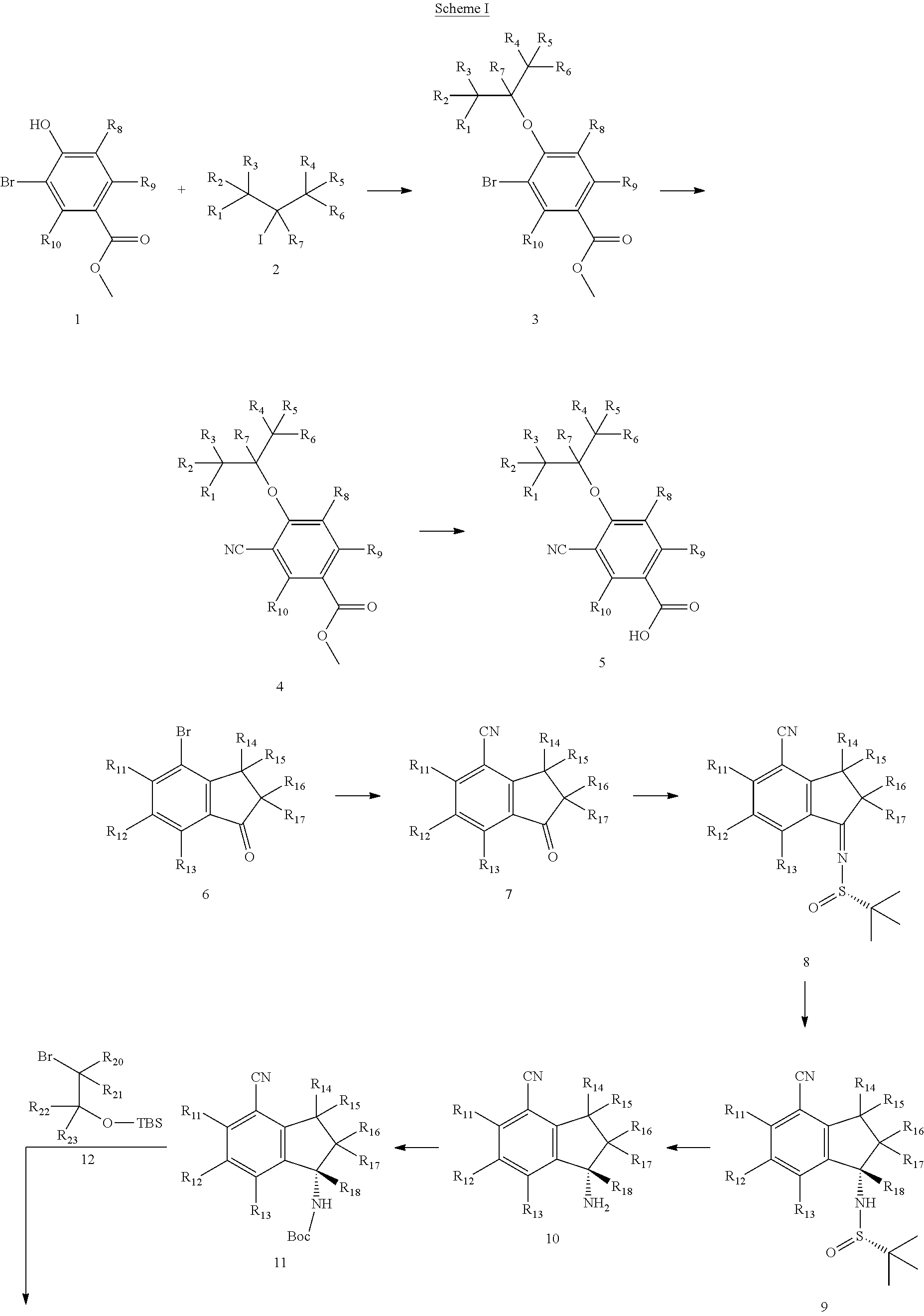

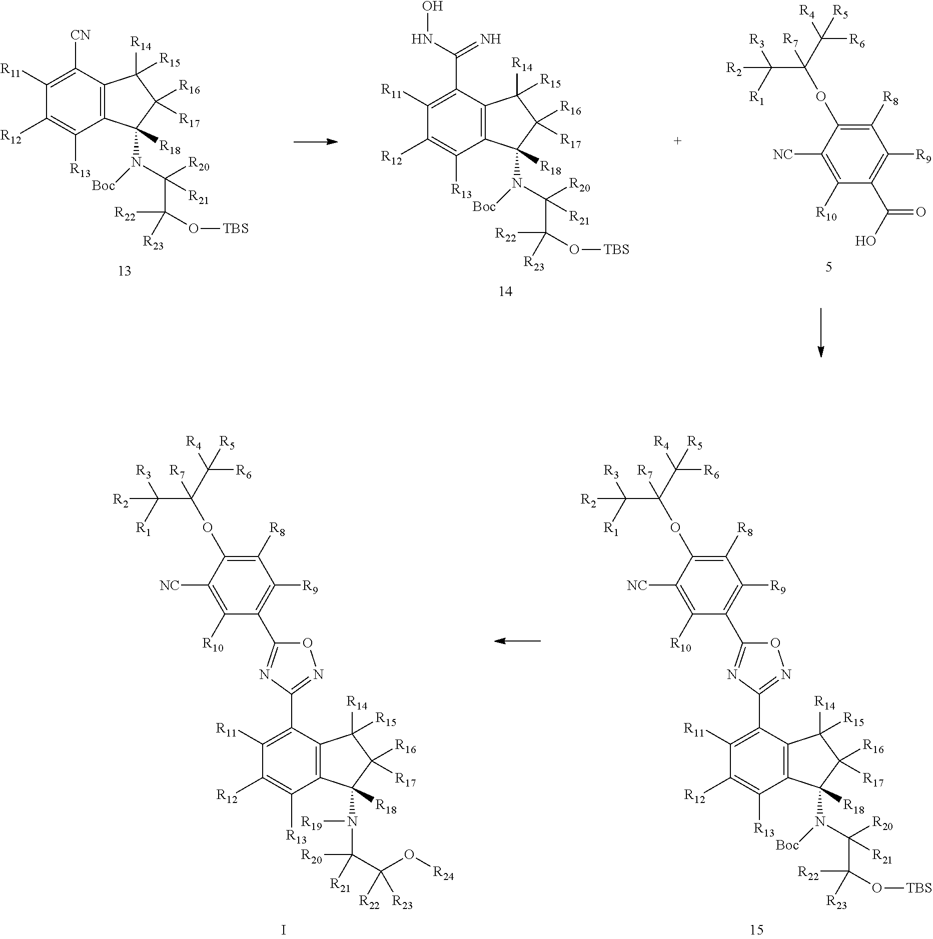

Compound 1 is reacted with compound 2 in the presence of an appropriate base, such as potassium carbonate, in an appropriate solvent, such as dimethylformamide, at an elevated temperature, to give compound 3. Compound 3 is treated with an appropriate cyanide salt, such as zinc chloride, in the presence of an appropriate catalyst, such as palladium (tetrakis) triphenylphosphine, in an appropriate solvent, such as N-methylpyrrolidine, at an elevated temperature, to give compound 4. Compound 4 is reacted with an appropriate base, such as sodium hydroxide, in an appropriate solvent, such as ethanol, to give compound 5. Compound 6 is treated with an appropriate cyanide salt, such as zinc chloride, in the presence of an appropriate catalyst, such as palladium (tetrakis) triphenylphosphine, in an appropriate solvent, such as N-methylpyrrolidine, at an elevated temperature, to give compound 7. Compound 7 is reacted with an appropriate hydroxylamine salt, such as hydroxylamine hydrochloride, in an appropriate solvent, such as ethanol, to give compound 7. Compound 7 is reacted with an appropriate chiral sulfinamide, such as (S)-2-methylpropane-2-sulfinamide, in the presence of an appropriate dehydrating agent, such as titanium tetraethoxide, in an appropriate solvent, such as toluene, to give compound 8. Compound 8 is treated with an appropriate reducing agent, such as sodium borohydride, in an appropriate solvent, such as tetrahydrofuran, at a reduced temperature, to give compound 9. Compound 9 is treated with an appropriate deprotecting agent, such as hydrogen chloride, in an appropriate solvent, such as a mixture of methanol and 1,4-dioxane, to give compound 10. Compound 10 is treated with an appropriate protecting agent, such as di-tert-butyl dicarbonate, in the presence of an appropriate base, such as trimethylamine, in an appropriate solvent, such as dichloromethane, to give compound 11 (where the abbreviation “Boc” refers to a tert-butylcarboxy group). Compound 11 is reacted with compound 12 (where the abbreviation “TBS” refers to a tert-butyldimethylsilyl group) in the presence of an appropriate base, such as sodium hydride, in an appropriate solvent, such as dimethylformamide, to give compound 13. Compound 13 is reacted with an appropriate hydroxylamine salt, such as hydroxylamine hydrochloride, in the presence of an appropriate base, such as triethylamine, in an appropriate solvent, such as ethanol, at an elevated temperature, to give compound 14. Compound 14 is reacted with compound 5 in the presence of an appropriate coupling agent, such as 1-ethyl-3-(3-dimethylaminopropyl)carbodiimide, in the presence of an appropriate additive, such as hydroxybenzotriazole, in an appropriate solvent, such as dimethylformamide, at an elevated temperature, to give compound 15. Compound 15 is treated with an appropriate deprotecting agent, such as hydrogen chloride, in an appropriate solvent, such as 1,4-dioxane, to give a compound of formula I.

Deuterium can be incorporated to different positions synthetically, according to the synthetic procedures as shown in Scheme I, by using appropriate deuterated intermediates. For example, to introduce deuterium at one or more positions of R₈-R₁₀, compound 1 with the corresponding deuterium substitutions can be used. To introduce deuterium at one or more positions of R₁-R₇, compound 2 with the corresponding deuterium substitutions can be used. To introduce deuterium at one or more positions of R₁₁-R₁₇, compound 6 with the corresponding deuterium substitutions can be used. To introduce deuterium at R₁₈, sodium cyanoborodeuteride can be used. To introduce deuterium at R₂₀-R₂₃, compound 5 with the corresponding deuterium substitutions can be used.

Deuterium can be incorporated to various positions having an exchangeable proton, such as the amine N—H and hydroxy O—H, via proton-deuterium equilibrium exchange. For example, to introduce deuterium at R₁₉ or R₂₄, these protons may be replaced with deuterium selectively or non-selectively through a proton-deuterium exchange method known in the art.

The invention is further illustrated by the following examples. All IUPAC names were generated using CambridgeSoft's ChemDraw.

### EXAMPLES

### Example 1

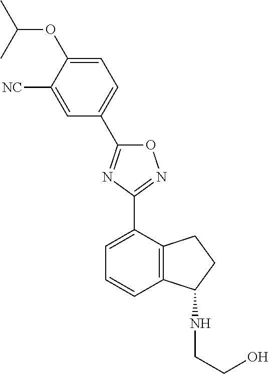 [SMILES: CC(C)Oc1ccc(-c2nc(-c3cccc4c3CC[C@@H]4NCCO)no2)cc1C#N]

Step A

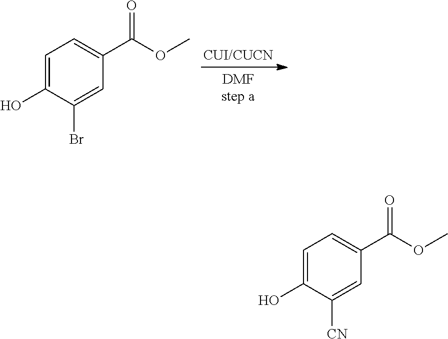

Methyl 3-cyano-4-hydroxybenzoate: To a solution of methyl 3-bromo-4-hydroxybenzoate (26 g, 112.53 mmol, 1.00 equiv) in DMF (160 mL) was added CuI (2.1 g, 11.11 mmol, 0.10 equiv) and CuCN (30 g, 337.08 mmol, 3.00 equiv). The resulting solution was stirred overnight at 120° C. The reaction mixture was cooled and diluted with water (300 mL). The resulting solution was extracted with dichloromethane (3×100 mL), and the organic layers were combined. The reaction mixture was dried over anhydrous sodium sulfate and concentrated under vacuum. The residue was purified by a silica gel column eluting with ethyl acetate/petroleum ether (1:1) to afford 13 g of methyl 3-cyano-4-hydroxybenzoate as a white solid.

Step B

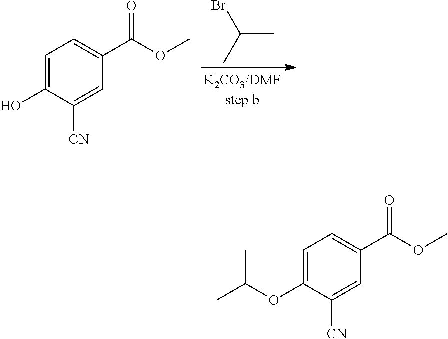

Methyl 3-cyano-4-(propan-2-yloxy)benzoate: To a solution of methyl 3-cyano-4-hydroxybenzoate (2 g, 11.29 mmol, 1.00 equiv) in DMF (20 mL) was added potassium carbonate (4.67 g, 33.79 mmol, 3.00 equiv). The resulting solution was stirred overnight at 80° C. The reaction mixture was cooled. The solids were filtered out. The pH value of the solution was adjusted to 9 with sodium hydroxide (0.5 Mol/L). The resulting solution was extracted with ether (2×25 mL), and the organic layers were combined. HCl (1 moL/L) was employed to adjust the pH value to 3. The resulting solution was extracted with ethyl acetate (2×25 mL), and the organic layers were combined, dried over anhydrous sodium sulfate and concentrated under vacuum to afford 1.6 g of methyl 3-cyano-4-(propan-2-yloxy)benzoate as white oil.

Step C

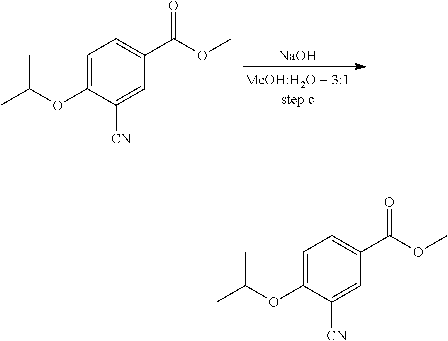

3-cyano-4-(propan-2-yloxy) benzoic acid: To a solution of methyl 3-cyano-4-(propan-2-yloxy)benzoate (5 g, 22.81 mmol, 1.00 equiv) in methanol/water (30:10 mL) was added sodium hydroxide (1.77 g, 2.30 equiv). The resulting solution was stirred for 2 h at 25° C. The pH value of the solution was adjusted to 3 with hydrogen chloride (1 Mol/L). The resulting solution was extracted with ethyl acetate (3×20 mL), and the organic layers were combined, dried over anhydrous sodium sulfate, concentrated under vacuum to afford 4.4 g of 3-cyano-4-(propan-2-yloxy) benzoic acid as a white solid.

Step 1

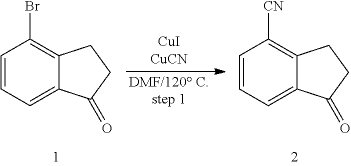 [SMILES: O=C1CCc2c(Br)cccc21 ; N#Cc1cccc2c1CCC2=O]

1-oxo-2,3-dihydro-1H-indene-4-carbonitrile: To a solution of 4-bromo-2,3-dihydro-1H-inden-1-one (50 g, 236.91 mmol, 1.00 equiv) in N,N-dimethylformamide (250 mL) was added CuCN (63.67 g, 710.92 mmol, 3.00 equiv), CuI (4.5 g, 23.63 mmol, 0.10 equiv). The resulting solution was stirred for 16 h at 120° C. The reaction mixture was cooled. The resulting solution was diluted with water (700 mL). The solids were filtered out. The resulting solution was extracted with ethyl acetate (4×300 mL) and the organic layers were combined, dried over anhydrous sodium sulfate and concentrated under vacuum. The residue was purified by a silica gel column eluting with ethyl acetate/petroleum ether (1:2) to afford 24 g (64%) of 1-oxo-2,3-dihydro-1H-indene-4-carbonitrile as a light brown solid.

Step 2

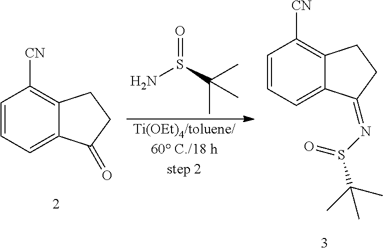

(S)—N-[(1Z)-4-cyano-2,3-dihydro-1H-inden-1-ylidene]-2-methylpropane-2-sulfinamide: To a solution of 1-oxo-2,3-dihydro-1H-indene-4-carbonitrile (5 g, 31.81 mmol, 1.00 equiv) in Toluene (60 mL) was added (S)-2-methylpropane-2-sulfinamide (4.24 g, 34.98 mmol, 1.10 equiv), Ti(OEt)₄ (10.75 g, 47.15 mmol, 1.48 equiv). The resulting solution was stirred for 16 h at 60° C. The resulting solution was used directly in the next step without further purification. LC-MS: m/z=261 [M+H]⁺.

Step 3

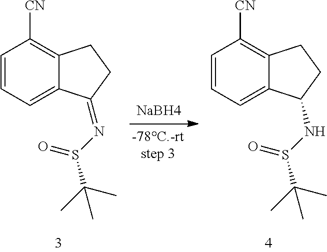 [SMILES: CC(C)(C)[S@](=O)/N=C1/CCc2c(C#N)cccc21 ; "NaBH4" ; CC(C)(C)[S@](=O)N[C@H]1CCc2c(C#N)cccc21]

(S)—N-[(1S)-4-cyano-2,3-dihydro-1H-inden-1-yl]-2-methylpropane-2-sulfinamide: A solution of (S)—N-[(1Z)-4-cyano-2,3-dihydro-1H-inden-1-ylidene]-2-methylpropane-2-sulfinamide (used directly from step 2) in tetrahydrofuran (80 mL) was cooled to −78° C. To this solution was added NaBH₄ (4.841 g, 127.97 mmol, 4.00 equiv) in portions over 30 min (the internal temperature did not rise during the addition). The resulting solution was stirred at −78° C. for 30 min and then warmed to 0° C. over 1 h. The reaction was placed in an ice bath and was quenched with brine (13 mL) and saturated sodium potassium tartrate (55 ml). The reaction mixture was diluted with ethyl acetate (200 ml) and was stirred at room temperature overnight. The organic layers were decanted and washed successively with saturated NH₄Cl, water, and brine. The organic layers were dried over MgSO₄ and filtered through a pad of MgSO₄. The filtrate was concentrated under vacuum to afford 4.56 g (55%) of (S)—N-[(1S)-4-cyano-2,3-dihydro-1H-inden-1-yl]-2-methylpropane-2-sulfinamide as a brown solid. LC-MS: m/z=263 [M+H]⁺.

Step 4

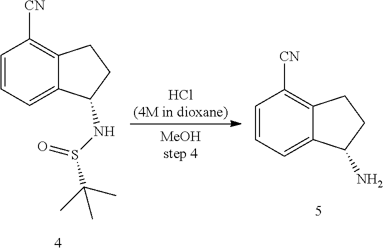 [SMILES: CC(C)(C)[S@](=O)N[C@H]1CCc2c(C#N)cccc21 ; "HCl(4M in dioxane)" ; N#Cc1cccc2c1CC[C@@H]2N]

(1S)-1-amino-2,3-dihydro-1H-indene-4-carbonitrile: To a solution of (S)—N-[(1S)-4-cyano-2,3-dihydro-1H-inden-1-yl]-2-methylpropane-2-sulfinamide (4.56 g, 17.38 mmol, 1.00 equiv) in methanol (18 mL) was added HCl (4 N in dioxane) (14 mL). The resulting solution was stirred for 1.5 h at room temperature. The resulting solution was diluted with methanol (40 mL). The solids were filtered out. The resulting mixture was concentrated under vacuum. The resulting solid was refluxed in acetonitrile (40 mL) and then cooled to room temperature. The solids was collected by filtration to afford 2.56 g (93%) of (1S)-1-amino-2,3-dihydro-1H-indene-4-carbonitrile as a light brown solid. LC-MS: m/z=159 [M+H]⁺.

Step 5

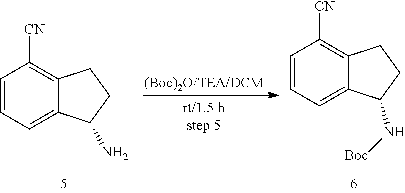 [SMILES: N#Cc1cccc2c1CC[C@@H]2N ; "(Boc)2O/TEA/DCM" ; CC(C)(C)OC(=O)N[C@H]1CCc2c(C#N)cccc21]

Tert-butyl N-[(1S)-4-cyano-2,3-dihydro-1H-inden-1-yl]carbamate: To a solution of (1S)-1-amino-2,3-dihydro-1H-indene-4-carbonitrile (2.56 g, 16.18 mmol, 1.00 equiv) in dichloromethane (20 mL) at 0° C. was added TEA (3.6 g, 35.58 mmol, 2.20 equiv), (BOc)₂O (3.89 g, 17.82 mmol, 1.10 equiv). The resulting solution was stirred for 1.5 h at room temperature. The resulting mixture was washed with brine, dried over anhydrous magnesium sulfate, filtered and concentrated. The residue was purified by a silica gel column eluting with ethyl acetate/petroleum ether (1:9) to afford 2.4 g (57%) of tert-butyl N-[(1S)-4-cyano-2,3-dihydro-1H-inden-1-yl]carbamate as an off-white solid. LC-MS: m/z=259 [M+H]⁺.

Step 6

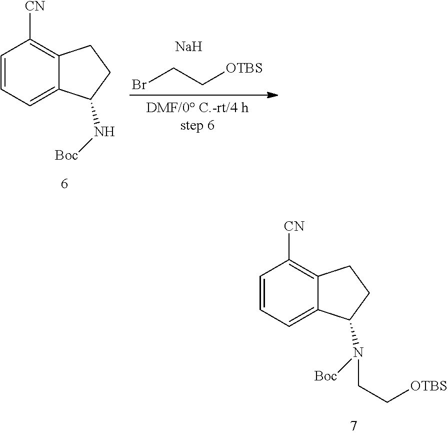

(1S)-4-cyano-2,3-dihydro-1H-inden-1-ylN-[2-[(tert-butyldimethylsilyl)oxy] ethyl] carbamate: To a solution of tert-butyl N-[(1S)-4-cyano-2,3-dihydro-1H-inden-1-yl]carbamate (1.7 g, 6.58 mmol, 1.00 equiv) in N,N-dimethylformamide (20 mL) was added sodium hydride (790 mg, 32.92 mmol, 3.00 equiv) at 0° C. The resulting solution was stirred at room temperature for 2 h. To this was added (2-bromoethoxy)(tert-butyl)dimethylsilane (3.14 g, 13.13 mmol, 2.00 equiv). The resulting solution was stirred for 3 h at room temperature. The reaction was then quenched by the addition of water/ice, extracted with ethyl acetate (3×50 mL) and the organic layers were combined. The resulting mixture was washed with brine (2×100 mL), dried over anhydrous sodium sulfate and concentrated under vacuum. The residue was purified by a silica gel column eluting with ethyl acetate/petroleum ether (1:20) to afford 1.47 g (53%) of tert-butyl (1S)-4-cyano-2,3-dihydro-1H-inden-1-yl N-[2-[(tert-butyldimethylsilyl)oxy]ethyl]carbamate as a light brown oil. LC-MS: m/z=417 [M+H]⁺.

Step 7

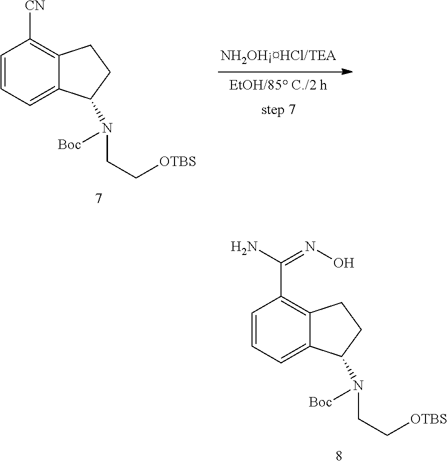 [SMILES: CC(C)(C)OC(=O)N(CCO[Si](C)(C)C(C)(C)C)[C@H]1CCc2c(C#N)cccc21 ; CC(C)(C)OC(=O)N(CCO[Si](C)(C)C(C)(C)C)[C@H]1CCc2c(/C(N)=N\O)cccc21]

Tert-butyl (1S)-4-[(E)-N′-hydroxycarbamimidoyl]-2,3-dihydro-1H-inden-1-yl N-[2-[(tert-butyldimethylsilyl)oxy]ethyl]carbamate: To a solution of tert-butyl (1S)-4-cyano-2,3-dihydro-1H-inden-1-yl N-[2-[(tert-butyldimethylsilyl)oxy]ethyl]carbamate (2.17 g, 5.20 mmol, 1.00 equiv) in ethanol (20 mL) was added NH₂OHHCl (1.08 g, 15.65 mmol, 3.00 equiv), TEA (1.58 g, 15.61 mmol, 3.00 equiv). The resulting solution was stirred for 2 h at 85° C. The reaction mixture was cooled. The resulting mixture was concentrated under vacuum. The resulting solution was diluted with water (40 mL), extracted with dichloromethane (3×40 mL). The organic layers were combined, dried over anhydrous sodium sulfate and concentrated under vacuum to afford 1.9 g (81%) of tert-butyl (1S)-4-[(E)-N′-hydroxycarbamimidoyl]-2,3-dihydro-1H-inden-1-yl N-[2-[(tert-butyldimethylsilyl)oxy]ethyl]carbamate as an off-white solid. LC-MS: m/z=450 [M+H]⁺.

Step 8

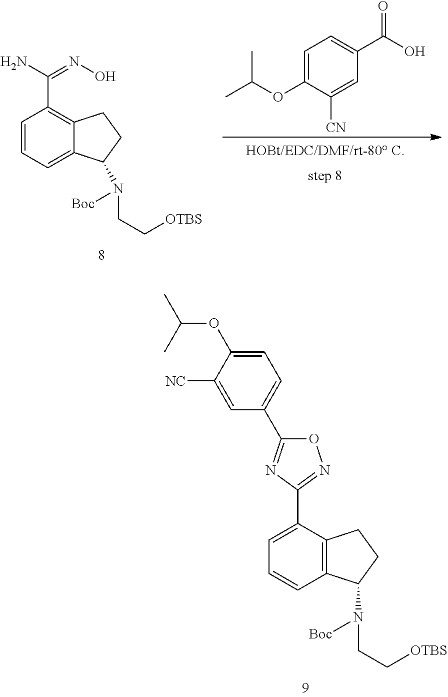 [SMILES: CC(C)(C)OC(=O)N(CCO[Si](C)(C)C(C)(C)C)[C@H]1CCc2c(/C(N)=N\O)cccc21 ; CC(C)Oc1ccc(C(=O)O)cc1C#N ; CC(C)Oc1ccc(-c2nc(-c3cccc4c3CC[C@@H]4N(CCO[Si](C)(C)C(C)(C)C)C(=O)OC(C)(C)C)no2)cc1C#N]

### tert-butyl (1S)-4-[5-[3-cyano-4-(propan-2-yloxy)phenyl]-1,2,4-oxadiazol-3-yl]-2,3-dihydro-1H-inden-1-yl-N-[2-[(tert-butyldimethylsilyl)oxy]ethyl]carbamate

To a solution of 3-cyano-4-(propan-2-yloxy)benzoic acid (787 mg, 3.84 mmol, 1.00 equiv) in N,N-dimethylformamide (20 mL) was added HOBT (674 mg, 4.99 mmol, 1.30 equiv), EDC (957 mg, 4.99 mmol, 1.30 equiv). The resulting solution was stirred at room temperature for 0.5 h. To this was added tert-butyl (1S)-4-[(E)-N′-hydroxycarbamimidoyl]-2,3-dihydro-1H-inden-1-yl-N-[2-[(tert-butyldimethylsilyl) oxy]ethyl]carbamate (1.9 g, 4.22 mmol, 1.10 equiv). The resulting solution was stirred at room temperature for 1 h then stirred overnight at 85° C. The reaction mixture was cooled and diluted with sodium bicarbonate. The resulting solution was extracted with ethyl acetate (3×100) and the organic layers were combined, dried over anhydrous sodium sulfate and concentrated under vacuum. The residue was purified by a silica gel column eluting with ethyl acetate/petroleum ether (1:5-1:3) to afford 1.6 g (67%) of tert-butyl (1S)-4-[5-[3-cyano-4-(propan-2-yloxy)phenyl]-1,2,4-oxadiazol-3-yl]-2,3-dihydro-1H-inden-1-yl-N-[2-[(tert-butyldimethylsilyl)oxy]ethyl]carbamate as light brown oil. LC-MS: m/z=619 [M+H]⁺.

Step 9

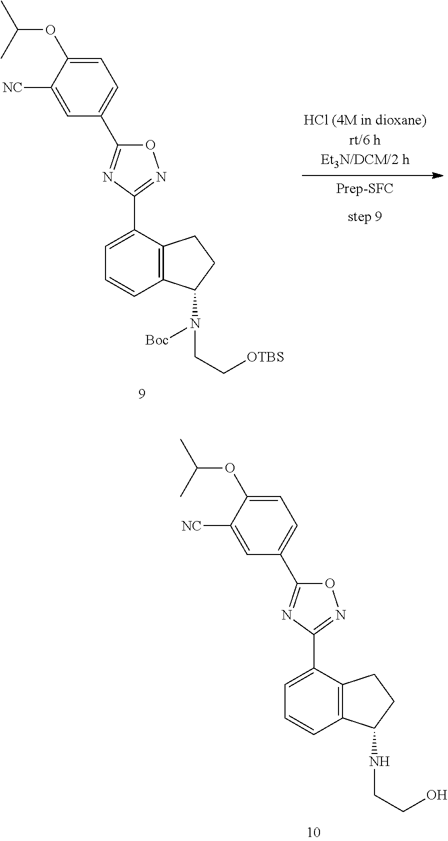 [SMILES: CC(C)Oc1ccc(-c2nc(-c3cccc4c3CC[C@@H]4N(CCO[Si](C)(C)C(C)(C)C)C(=O)OC(C)(C)C)no2)cc1C#N ; CC(C)Oc1ccc(-c2nc(-c3cccc4c3CC[C@@H]4NCCO)no2)cc1C#N]

5-[3-[(1S)-1-[(2-hydroxyethyl)amino]-2,3-dihydro-1H-inden-4-yl]-1,2,4-oxadiazol-5-yl]-2-(propan-2-yloxy)benzonitrile: To a solution of tert-butyl N-[2-[(tert-butyldimethylsilyl)oxy]ethyl]-N-[(1S)-4-[5-[3-cyano-4-(propan-2-yloxy)phenyl]-1,2,4-oxadiazol-3-yl]-2,3-dihydro-1H-inden-1-yl]carbamate (500 mg, 0.81 mmol, 1.00 equiv) was added HCl (4M in dioxane)(10 mL). The resulting solution was stirred at room temperature for 6 h. The solid was filtered out and dissolved in DCM (10 mL). To this was added triethylamine (245 mg, 2.43 mmol, 3.00 equiv). The resulting solution was stirred for 2 h at room temperature and then washed by water (2×20 mL). The organic layers were dried over anhydrous sodium sulfate and concentrated under vacuum. The crude product was purified by Prep-SFC with the following conditions: Column: Phenomenex Lux 5u Cellulose-4, AXIA Packed, 250*21.2 mm, Sum; Mobile Phase A: CO₂:50, Mobile Phase B: MeOH (0.2% DEA):50; Flow rate: 50 mL/min; 220 nm; RT: 6.12 to afford 133.6 mg (41%) of 5-[3-[(1S)-1-[(2-hydroxyethyl)amino]-2,3-dihydro-1H-inden-4-yl]-1,2,4-oxadiazol-5-yl]-2-(propan-2-yloxy)benzonitrile as a white solid.

¹H NMR (300 MHz, CDCl₃) δ 8.43-8.27 (m, 2H), 8.11-8.01 (m, 1H), 7.52 (d, J=7.5 Hz, 1H), 7.37 (t, J=7.6 Hz, 1H), 7.12 (d, J=9.0 Hz, 1H), 4.80 (m, 1H), 4.33 (m, 1H), 3.77-3.60 (m, 2H), 3.44 (m, 1H), 3.26-3.09 (m, 1H), 3.01-2.83 (m, 2H), 2.51 (m, 1H), 2.21 (brs, 2H), 1.91 (m, 1H), 1.48 (d, J=6.0 Hz, 6H). LC-MS: m/z=405[M+H]⁺.

### Example 2

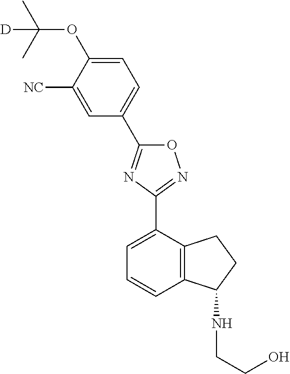 [SMILES: [2H]C(C)(C)Oc1ccc(-c2nc(-c3cccc4c3CC[C@@H]4NCCO)no2)cc1C#N]

Step 1

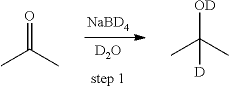

(2-²H)propan-2-(²H)ol: To a solution of propan-2-one (15 g, 258.27 mmol, 1.00 equiv) in D₂O (50 mL) was added NaBD₄ (5.4 g, 128.57 mmol, 0.50 equiv) in portions at 0° C. in 20 min. To this solution was added AcCl (5.2 g, 66.67 mmol, 0.26 equiv). The resulting solution was stirred for 2 h at 25° C. The reaction progress was monitored by GCMS. The reaction was then quenched by the addition of AcCl (5.2 g) dropwise at 0° C. in 20 min. The crude product was purified by distillation under reduced pressure (760 mm Hg) and the fraction was collected at 75-90° C. to afford 22 g (crude) of (2-²H)propan-2-(²H)ol as a colorless liquid.

Step 2

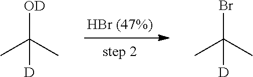

2-bromo(2-²H)propane: A solution of (2-²H)propan-2-(²H)ol (22 g, 354.23 mmol, 1.00 equiv) in HBr (47% in H₂O) (50 mL) was stirred for 3 h at 80° C. The reaction progress was monitored by GCMS. The crude product was purified by distillation under normal pressure and the fraction was collected at 70-80° C. to afford 22 g (50%) of 2-bromo(2-²H)propane as a colorless liquid.

Step 3

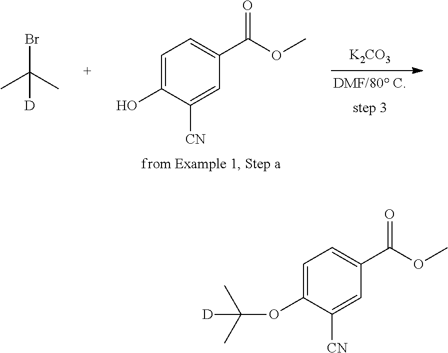

3-cyano-4-[(2-²H)propan-2-yloxy]benzoate: To a solution of methyl 3-cyano-4-hydroxybenzoate (5.5 g, 31.05 mmol, 1.00 equiv) in N,N-dimethylformamide (60 mL) were added 2-bromo(2-²H)propane (7.7 g, 62.10 mmol, 2.00 equiv), potassium carbonate (12.77 g, 92.40 mmol, 3.00 equiv). The resulting solution was stirred overnight at 80° C. The reaction progress was monitored by HNMR. The reaction mixture was cooled. The solids were filtered out. The pH value of the solution was adjusted to 9 with sodium hydroxide (0.5 mol/L). The resulting solution was extracted with ethyl acetate (3×30 mL) and the organic layers were combined. HCl (1 mol/L) was employed to adjust the pH to 3. The resulting solution was extracted with ethyl acetate (2×30 mL) and the organic layers were combined, dried over anhydrous sodium sulfate and concentrated under vacuum to afford 6.0 g of methyl 3-cyano-4-[(2-²H)propan-2-yloxy]benzoate as light yellow oil.

Step 4

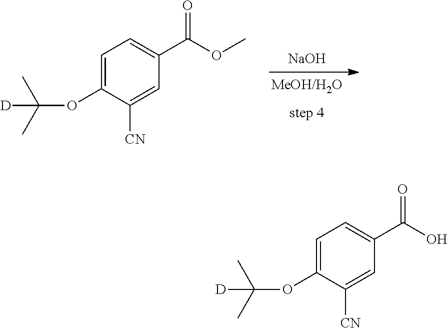

3-cyano-4-[(2-²H)propan-2-yloxy]benzoic acid: To a solution of methyl 3-cyano-4-[(2-²H)propan-2-yloxy]benzoate (6.1 g, 27.70 mmol, 1.00 equiv) in methanol/water (30:10 mL) was added sodium hydroxide (2.22 g, 55.50 mmol, 2.00 equiv). The resulting solution was stirred for 2 h at 25° C. The reaction progress was monitored by H NMR. The pH value of the solution was adjusted to 3 with hydrogen chloride (1 mol/L). The solids were collected by filtration to afford 5.0 g of 3-cyano-4-[(2-²H)propan-2-yloxy]benzoic acid as a white solid.

Step 5

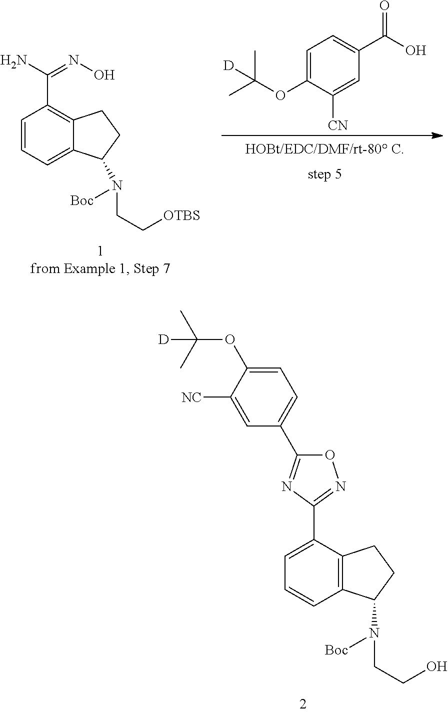 [SMILES: CC(C)(C)OC(=O)N(CCO[Si](C)(C)C(C)(C)C)[C@H]1CCc2c(/C(N)=N\O)cccc21 ; [2H]C(C)(C)Oc1ccc(C(=O)O)cc1C#N ; [2H]C(C)(C)Oc1ccc(-c2nc(-c3cccc4c3CC[C@@H]4N(CCO)C(=O)OC(C)(C)C)no2)cc1C#N]

tert-butyl (1S)-4-(5-[3-cyano-4-[(2-²H)propan-2-yloxy]phenyl]-1,2,4-oxadiazol-3-yl)-2,3-dihydro-1H-inden-1-ylN-(2-hydroxyethyl)carbamate: To a solution of 3-cyano-4-[(2-²H)propan-2-yloxy]benzoic acid (460 mg, 2.23 mmol, 1.00 equiv) in N,N-dimethylformamide (10 mL) was added HOBT (390 mg, 2.89 mmol, 1.30 equiv), EDC(HCl) (560 mg, 2.92 mmol, 1.30 equiv). The resulting solution was stirred at room temperature for 0.5 h and then tert-butyl (1S)-4-[(E)-N′-hydroxycarbamimidoyl]-2,3-dihydro-1H-inden-1-yl-N-[2-[(tert-butyldimethylsilyl) oxy]ethyl]carbamate (1 g, 2.22 mmol, 1.00 equiv) was added. The resulting solution was stirred at room temperature for 1 h then stirred overnight at 80° C. The reaction mixture was cooled and diluted with water, extracted with ethyl acetate (3×50 mL). The organic layers were combined and dried over anhydrous sodium sulfate and concentrated under vacuum. The residue was purified by a silica gel column eluting with ethyl acetate/petroleum ether (1:5-1:3) to afford 0.83 g (73%) of tert-butyl (1S)-4-(5-[3-cyano-4-[(2-²H)propan-2-yloxy]phenyl]-1,2,4-oxadiazol-3-yl)-2,3-dihydro-1H-inden-1-yl N-(2-hydroxyethyl)carbamate as light brown oil.

Step 6

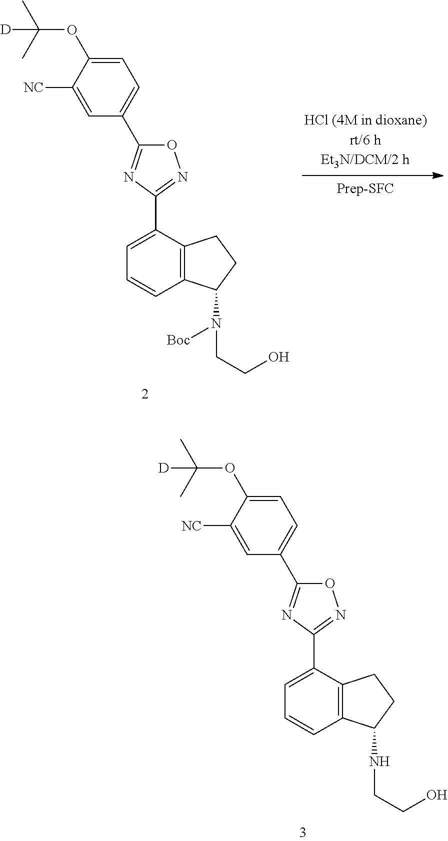 [SMILES: [2H]C(C)(C)Oc1ccc(-c2nc(-c3cccc4c3CC[C@@H]4N(CCO)C(=O)OC(C)(C)C)no2)cc1C#N ; [2H]C(C)(C)Oc1ccc(-c2nc(-c3cccc4c3CC[C@@H]4NCCO)no2)cc1C#N]

5-[3-[(1S)-1-[(2-hydroxyethyl)amino]-2,3-dihydro-1H-inden-4-yl]-1,2,4-oxadiazol-5-yl]-2-[(2-²H)propan-2-yloxy]benzonitrile: To a solution of tert-butyl N-[(1S)-4-(5-[3-cyano-4-[(2-²H)propan-2-yloxy]phenyl]-1,2,4-oxadiazol-3-yl)-2,3-dihydro-1H-inden-1-yl]-N-(2-hydroxyethyl)carbamate (500 mg, 0.99 mmol, 1.00 equiv) was added hydrogen chloride (4M in dioxane) (10 mL). The resulting solution was stirred at room temperature for 6 h. The solid was filtered out and dissolved in DCM (10 mL). Then triethylamine (300 mg, 2.97 mmol, 3.00 equiv) was added. The resulting solution was stirred for 2 h at room temperature. The resulting solution was washed by water (2×20 mL), dried over anhydrous sodium sulfate and concentrated under vacuum. The crude product was purified by Prep-SFC with the following conditions: Column: Phenomenex Lux 5u Cellulose-4, AXIA Packed, 250*21.2 mm, Sum; Mobile Phase A: CO₂:50, Mobile Phase B: MeOH (0.2% DEA):50; Flow rate: 50 mL/min; 220 nm; RT: 6.12 to afford 107.2 mg (27%) of 5-[3-[(1S)-1-[(2-hydroxyethyl)amino]-2,3-dihydro-1H-inden-4-yl]-1,2,4-oxadiazol-5-yl]-2-[(2-²H)propan-2-yloxy]benzonitrile as a white solid.

¹H NMR (300 MHz, CDCl₃) δ 8.45-8.28 (m, 2H), 8.16-8.03 (m, 1H), 7.55 (d, J=7.5 Hz, 1H), 7.38 (t, J=7.6 Hz, 1H), 7.12 (d, J=8.9 Hz, 1H), 4.35 (m, 1H), 3.70 (m, 2H), 3.46 (m, 1H), 3.28-3.10 (m, 1H), 3.03-2.83 (m, 2H), 2.52 (m, 1H), 2.21 (brs, 2H), 1.92 (m, 1H), 1.47 (s, 6H). LC-MS: m/z=406[M+H]⁺.

### Example 3

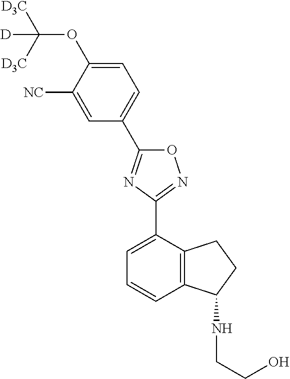 [SMILES: [2H]C([2H])([2H])C([2H])(Oc1ccc(-c2nc(-c3cccc4c3CC[C@@H]4NCCO)no2)cc1C#N)C([2H])([2H])[2H]]

Step 1

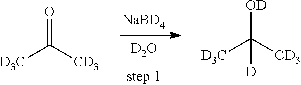

(²H₇)propan-2-(²H)ol: To a solution of (²H₆)propan-2-one (15 g, 233.95 mmol, 1.00 equiv) in D₂O (30 mL) was added NaBD₄ (4.9 g, 116.67 mmol, 0.50 equiv) in portions at 0° C. in 20 min. The resulting solution was stirred for 2 h at 25° C. The reaction progress was monitored by GCMS. The reaction was then quenched by the addition of AcCl (4.75 g, 60.90 mmol, 0.26 equiv) dropwise with stirring at 0° C. in 20 min. The crude product was distilled under normal pressure and the fraction was collected at 75-95° C. to afford 20 g (crude) of (²H₇)propan-2-(²H)ol as colorless oil.

Step 2

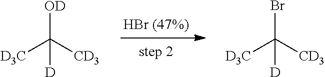

2-bromo(²H₇)propane: To a solution of (²H₇)propan-2-(²H)ol (20 g, 293.49 mmol, 1.00 equiv), HBr (47% in H₂O) (50 mL). The resulting solution was stirred for 3 h at 80° C. in an oil bath. The reaction progress was monitored by GCMS. The crude product was purified by distillation under normal pressure (760 mm Hg) and the fraction was collected at 70-80° C. to afford 19 g (50%) of 2-bromo(²H₇)propane as a colorless liquid.

Step 3

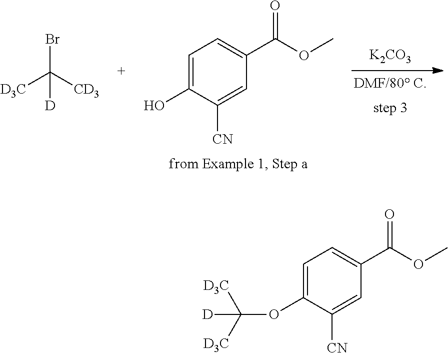

Methyl 3-cyano-4-[(²H₇)propan-2-yloxy]benzoate: To a solution of methyl 3-cyano-4-hydroxybenzoate (2.0 g, 11.29 mmol, 1.00 equiv) in N,N-dimethylformamide (20 mL) were added 2-bromo(²H₇)propane (3.0 g, 23.07 mmol, 2.00 equiv), potassium carbonate (4.67 g, 3.00 equiv). The resulting solution was stirred overnight at 80° C. The reaction progress was monitored by HNMR. The reaction mixture was cooled. The solids were filtered out. The pH value of the solution was adjusted to 9 with sodium hydroxide (0.5 mol/L). The resulting solution was extracted with ethyl acetate (3×30 mL) and the organic layers were combined. Hydrogen chloride (1 mol/L) was employed to adjust the pH to 3. The resulting solution was extracted with ethyl acetate (2×30 mL) and the organic layers were combined, dried over anhydrous sodium sulfate and concentrated under vacuum to afford 1.8 g (70%) of methyl 3-cyano-4-[(²H₇)propan-2-yloxy]benzoate as light yellow oil.

Step 4

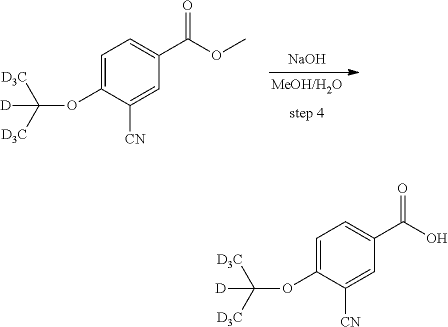

3-cyano-4-[(²H₇)propan-2-yloxy]benzoic acid: To a solution of methyl 3-cyano-4-[(²H₇)propan-2-yloxy]benzoate (5.5 g, 24.31 mmol, 1.00 equiv) in methanol/water (30:10 mL) was added sodium hydroxide (1.94 g, 2.00 equiv). The resulting solution was stirred for 2 h at 25° C. The reaction progress was monitored by HNMR. The pH value of the solution was adjusted to 3 with hydrogen chloride (1 mol/L). The solids were collected by filtration to afford 4.9 g of 3-cyano-4-[(²H₇)propan-2-yloxy]benzoic acid as a white solid.

Step 5

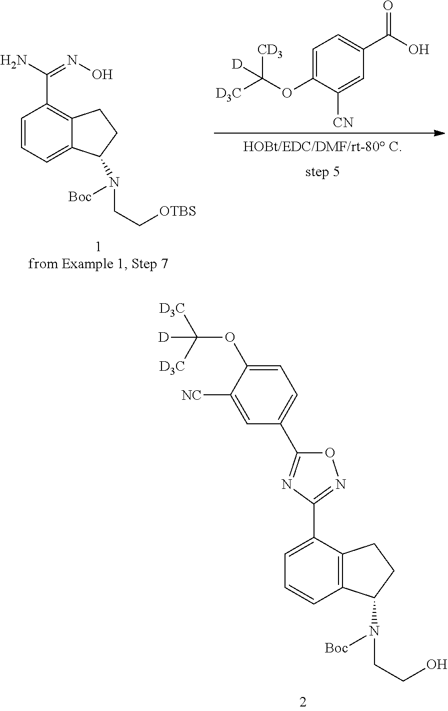 [SMILES: CC(C)(C)OC(=O)N(CCO[Si](C)(C)C(C)(C)C)[C@H]1CCc2c(/C(N)=N\O)cccc21 ; [2H]C([2H])([2H])C([2H])(Oc1ccc(C(=O)O)cc1C#N)C([2H])([2H])[2H] ; [2H]C([2H])([2H])C([2H])(Oc1ccc(-c2nc(-c3cccc4c3CC[C@@H]4N(CCO)C(=O)OC(C)(C)C)no2)cc1C#N)C([2H])([2H])[2H]]

Tert-butyl (1S)-4-(5-[3-cyano-4-[(²H₇)propan-2-yloxy]phenyl]-1,2,4-oxadiazol-3-yl)-2,3-dihydro-1H-inden-1-yl N-(2-hydroxyethyl)carbamate: To a solution of 3-cyano-4-[(²H₇)propan-2-yloxy]benzoic acid (470 mg, 2.21 mmol, 1.00 equiv) in N,N-dimethylformamide (10 mL) were added HOBt (390 mg, 2.89 mmol, 1.30 equiv), EDC(HCl) (550 mg, 2.87 mmol, 1.30 equiv). The resulting solution was stirred at room temperature for 0.5 h. To this was added tert-butyl (1S)-4-[(E)-N′-hydroxycarbamimidoyl]-2,3-dihydro-1H-inden-1-yl N-[2-[(tert-butyldimethylsilyl)oxy]ethyl]carbamate (1 g, 2.22 mmol, 1.00 equiv). The resulting solution was stirred at room temperature for 1 h then stirred overnight at 80° C. The reaction mixture was cooled and diluted with water, extracted with ethyl acetate (3×50 mL) and the organic layers were combined, dried over anhydrous sodium sulfate and concentrated under vacuum. The residue was purified by a silica gel column eluting with ethyl acetate/petroleum ether (1:5-1:3) to afford 0.83 g (73%) of tert-butyl (1S)-4-(5-[3-cyano-4-[(²H₇)propan-2-yloxy]phenyl]-1,2,4-oxadiazol-3-yl)-2,3-dihydro-1H-inden-1-yl N-(2-hydroxyethyl)carbamate as light brown oil. LC-MS: m/z=512[M+H]⁺.

Step 6

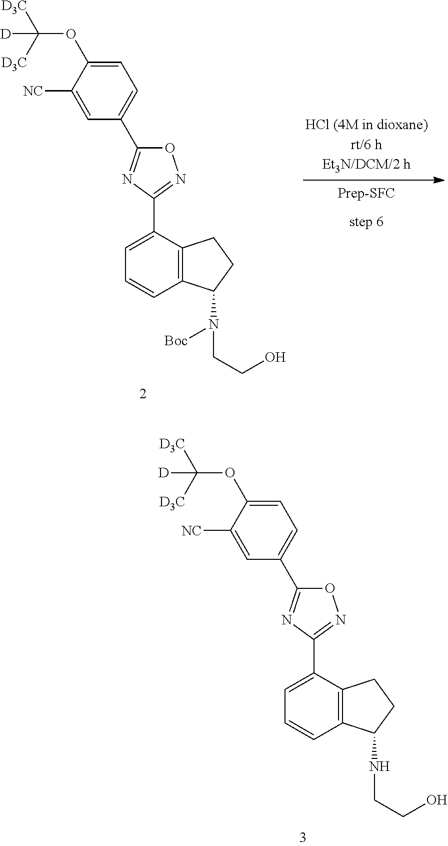 [SMILES: [2H]C([2H])([2H])C([2H])(Oc1ccc(-c2nc(-c3cccc4c3CC[C@@H]4N(CCO)C(=O)OC(C)(C)C)no2)cc1C#N)C([2H])([2H])[2H] ; [2H]C([2H])([2H])C([2H])(Oc1ccc(-c2nc(-c3cccc4c3CC[C@@H]4NCCO)no2)cc1C#N)C([2H])([2H])[2H]]

5-[3-[(1S)-1-[(2-hydroxyethyl)amino]-2,3-dihydro-1H-inden-4-yl]-1,2,4-oxadiazol-5-yl]-2-[(²H₇)propan-2-yloxy]benzonitrile: To a solution of tert-butyl N-[(1S)-4-(5-[3-cyano-4-[(²H₇)propan-2-yloxy]phenyl]-1,2,4-oxadiazol-3-yl)-2,3-dihydro-1H-inden-1-yl]-N-(2-hydroxyethyl)carbamate (500 mg, 0.98 mmol, 1.00 equiv) was added HCl (4M in dioxane) (10 mL). The resulting solution was stirred at room temperature for 6 h. The solid was collected by filtration and dissolved in DCM (10 mL). Triethylamine (296 mg, 2.93 mmol, 3.00 equiv) was added and the resulting solution was stirred for 2 h at room temperature. The resulting solution was washed with water (2×20 mL), dried over anhydrous sodium sulfate and concentrated under vacuum. The crude product was purified by Prep-SFC with the following conditions: Column: Phenomenex Lux 5u Cellulose-4, AXIA Packed, 250*21.2 mm, Sum; Mobile Phase A: CO₂:50, Mobile Phase B: MeOH (0.2% DEA):50; Flow rate: 50 mL/min; 220 nm; RT: 6.12. This resulted in 111.9 mg (28%) of 5-[3-[(1S)-1-[(2-hydroxyethyl)amino]-2,3-dihydro-1H-inden-4-yl]-1,2,4-oxadiazol-5-yl]-2-[(²H₇)propan-2-yloxy]benzonitrile as a white solid. 1H NMR (300 MHz, CDCl₃) δ 8.44-8.27 (m, 2H), 8.11-8.02 (m, 1H), 7.53 (d, J=7.4 Hz, 1H), 7.37 (t, J=7.6 Hz, 1H), 7.12 (d, J=8.9 Hz, 1H), 4.33 (t, J=6.8 Hz, 1H), 3.79-3.60 (m, 2H), 3.44 (m, 1H), 3.18 (m, 1H), 3.02-2.82 (m, 2H), 2.51 (m, 1H), 2.17 (brs, 2H), 1.91 (m, 1H). LC-MS: m/z=412[M+H]⁺.

### Example 4

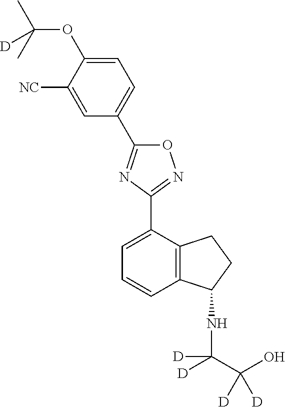 [SMILES: [2H]C(C)(C)Oc1ccc(-c2nc(-c3cccc4c3CC[C@@H]4NC([2H])([2H])C([2H])([2H])O)no2)cc1C#N]

Step 1

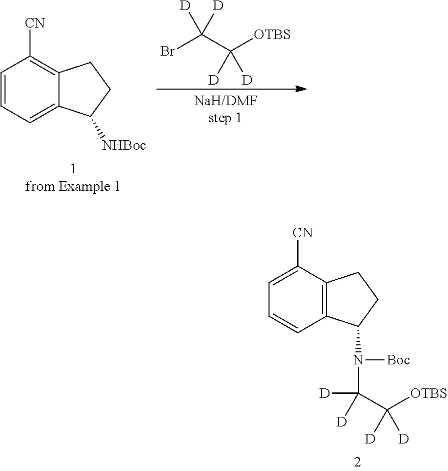 [SMILES: CC(C)(C)OC(=O)N[C@H]1CCc2c(C#N)cccc21 ; [2H]C([2H])(Br)C([2H])([2H])O[Si](C)(C)C(C)(C)C ; [2H]C([2H])(O[Si](C)(C)C(C)(C)C)C([2H])([2H])N(C(=O)OC(C)(C)C)[C@H]1CCc2c(C#N)cccc21]

Tert-butylN-[2-[(tert-butyldimethylsilyl)oxy](²H₄)ethyl]-N-[(1S)-4-cyano-2,3-dihydro-1H-inden-1-yl]carbamate: To a solution of tert-butyl N-[(1S)-4-cyano-2,3-dihydro-1H-inden-1-yl]carbamate (1.9 g, 7.36 mmol, 1.00 equiv) in DMF (20 mL) was added sodium hydride (880 mg, 3.00 equiv) at 0° C. The resulting solution was stirred for 2 h at room temperature. Then [2-bromo(²H₄)ethoxy](tert-butyl)dimethylsilane (3.6 g, 14.80 mmol, 2.00 equiv) was added. The resulting solution was stirred for 3 h at room temperature. The reaction was then quenched by the addition of water/ice, extracted with 3×100 mL of ethyl acetate and the organic layers were combined. The mixture was dried over anhydrous sodium sulfate and concentrated under vacuum. The crude product was purified by Sift chromatography, eluted with ethyl acetate/petroleum ether (1:20) to afford 1.94 g (63%) of tert-butylN-[2-[(tert-butyldimethylsilyl)oxy](²H₄)ethyl]-N-[(1S) 4-cyano-2,3-dihydro-1H-inden-1-yl]carbamate as yellow oil. LC-MS: m/z=421 [M+H]⁺.

Step 2

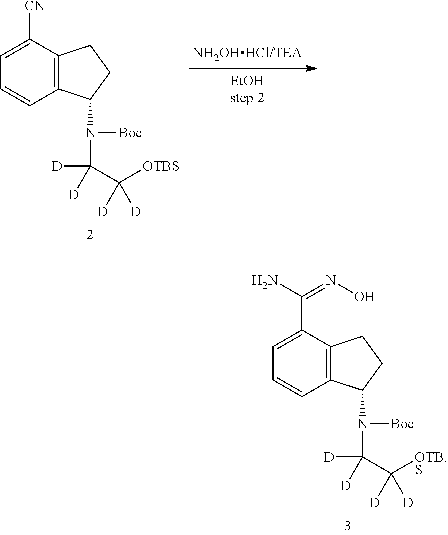

tert-butylN-[2-[(tert-butyldimethylsilyl)oxy](²H₄)ethyl]-N-[(1S)-4-[(E)-N′-hydroxycarbamimidoyl]-2,3-dihydro-1H-inden-1-yl]carbamate: To a solution of tert-butyl N-[2-[(tert-butyldimethylsilyl)oxy](²H₄)ethyl]-N-[(1S)-4-cyano-2,3-dihydro-1H-inden-1-yl]carbamate (1.94 g, 4.61 mmol, 1.00 equiv) in ethanol (20 mL) were added NH₂OH.HCl (0.96 g, 3.00 equiv) and TEA (1.40 g, 13.84 mmol, 3.00 equiv). The resulting solution was stirred for 2 h at 85° C. The reaction progress was monitored by LCMS. Then the resulting solution was concentrated under vacuum to remove ethanol. The residue was diluted with water (20 mL) and extracted with DCM (3×20 mL), dried over anhydrous sodium sulfate, filtered and concentrated under vacuum to afford 1.9 g (91%) of tert-butyl N-[2-[(tert-butyldimethylsilyl)oxy](²H₄)ethyl]-N-[(1S)-4-[(E)-N′-hydroxycarbamimidoyl]-2,3-dihydro-1H-inden-1-yl]carbamate as light yellow oil. LC-MS: m/z=454 [M+H]⁺.

Step 3

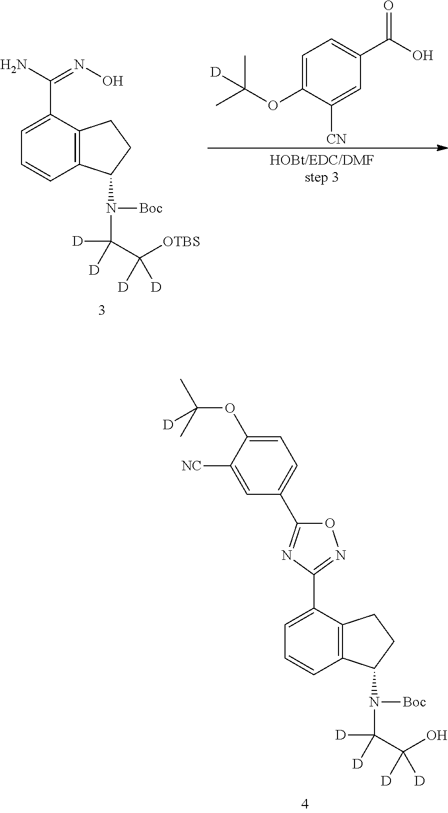 [SMILES: [2H]C([2H])(O[Si](C)(C)C(C)(C)C)C([2H])([2H])N(C(=O)OC(C)(C)C)[C@H]1CCc2c(/C(N)=N\O)cccc21 ; [2H]C(C)(C)Oc1ccc(C(=O)O)cc1C#N ; [2H]C(C)(C)Oc1ccc(-c2nc(-c3cccc4c3CC[C@@H]4N(C(=O)OC(C)(C)C)C([2H])([2H])C([2H])([2H])O)no2)cc1C#N]

tert-butylN-[(1S)-4-(5-[3-cyano-4-[(2-²H)propan-2-yloxy]phenyl]-1,2,4-oxadiazol-3-yl)-2,3-dihydro-1H-inden-1-yl]-N-[2-hydroxy(²H₄)ethyl]carbamate: To a solution of 3-cyano-4-[(2-²H)propan-2-yloxy]benzoic acid (546 mg, 2.65 mmol, 1.00 equiv) in DMF (15 mL) were added HOBT (465 mg, 3.44 mmol, 1.30 equiv) and EDC (660 mg, 4.25 mmol, 1.30 equiv). The mixture was stirred for 30 min at room temperature. Then tert-butylN-[2-[(tert-butyldimethylsilyl)oxy](²H₄)ethyl]-N-[(1S)-4-[(E)-N′-hydroxycarbamimidoyl]-2,3-dihydro-1H-inden-1-yl]carbamate (1.2 g, 2.65 mmol, 1.00 equiv) was added. The mixture solution was allowed to stir for 1 hour at room temperature. Then the resulting solution was stirred at 80° C. overnight. The reaction was diluted with water (20 mL) and extracted with ethyl acetate (3×30 mL), dried over anhydrous sodium sulfate. The crude product was purified by Sift chromatography eluted with ethyl acetate/petroleum ether (3:7) to afford 1 g (74%) of tert-butyl N-[(1S)-4-(5-[3-cyano-4-[(2-²H)propan-2-yloxy] phenyl]-1,2,4-oxadiazol-3-yl)-2,3-dihydro-1H-inden-1-yl]-N-[2-hydroxy(²H₄)ethyl]carbamate as light yellow oil. LC-MS: m/z=510 [M+H]⁺.

Step 4

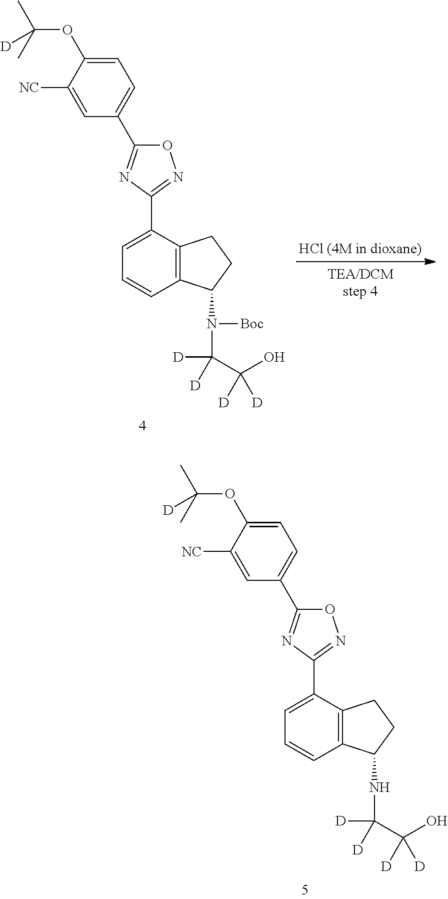 [SMILES: [2H]C(C)(C)Oc1ccc(-c2nc(-c3cccc4c3CC[C@@H]4N(C(=O)OC(C)(C)C)C([2H])([2H])C([2H])([2H])O)no2)cc1C#N ; [2H]C(C)(C)Oc1ccc(-c2nc(-c3cccc4c3CC[C@@H]4NC([2H])([2H])C([2H])([2H])O)no2)cc1C#N]

5-[3-[(1S)-1-[[2-hydroxy(²H₄)ethyl]amino]-2,3-dihydro-1H-inden-4-yl]-1,2,4-oxadiazol-5-yl]-2-[(2-²H)propan-2-yloxy]benzonitrile: A solution of tert-butyl N-[(1S)-4-(5-[3-cyano-4-[(2-²H)propan-2-yloxy]phenyl]-1,2,4-oxadiazol-3-yl)-2,3-dihydro-1H-inden-1-yl]-N-[2-hydroxy(²H₄)ethyl]carbamate (500 mg, 0.98 mmol, 1.00 equiv) in hydrogen chloride (4 M in dioxane) (10 mL) was stirred for 6 h at room temperature. The mixture was filtered to obtain 325 mg of white solid. Then white solid was suspended in DCM (10 mL) and TEA (241 mg, 3.00 equiv) was added. The resulting solution was stirred for 2 h at room temperature. The solution was washed by water (2×20 mL), dried over anhydrous sodium sulfate. The crude product was purified by Flash-Prep-HPLC with the following conditions: Column, XBridge Shield RP18 OBD Column, Sum, 19*150 mm; mobile phase, Water (10 mmol/L NH₄HCO₃) and ACN- (25.0% up to 55.0% in 7 min); Detector, UV 254 & 220 nm to afford 180 mg (45%) of 5-[3-[(1S)-1-[[2-hydroxy²H₄)ethyl]amino]-2,3-dihydro-1H-inden-4-yl]-1,2,4-oxadiazol-5-yl]-2-[(2-²H)propan-2-yloxy]benzonitrile as a white solid. ¹H NMR (300 MHz, CDCl₃) δ 8.39-8.38 (m, 1H), 8.34-8.30 (m, 1H), 8.07-8.00 (m, 1H), 7.53-7.51 (m, 1H), 7.39-7.34 (m, 1H), 7.13-7.10 (m, 1H), 4.34-4.30 (m, 1H), 3.48-3.38 (m, 1H), 3.22-3.11 (m, 1H), 2.56-2.45 (m, 1H), 2.26 (s, 2H), 1.96-1.88 (m, 1H), 1.47 (s, 6H). LC-MS: m/z=410 [M+H]⁺.

### Example 5

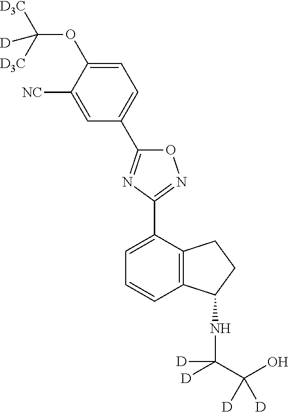 [SMILES: [2H]C([2H])([2H])C([2H])(Oc1ccc(-c2nc(-c3cccc4c3CC[C@@H]4NC([2H])([2H])C([2H])([2H])O)no2)cc1C#N)C([2H])([2H])[2H]]

Step 1

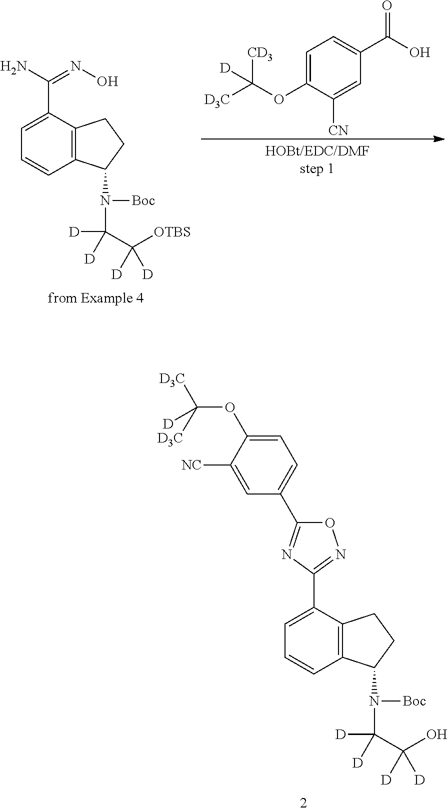 [SMILES: [2H]C([2H])(O[Si](C)(C)C(C)(C)C)C([2H])([2H])N(C(=O)OC(C)(C)C)[C@H]1CCc2c(/C(N)=N\O)cccc21 ; [2H]C([2H])([2H])C([2H])(Oc1ccc(C(=O)O)cc1C#N)C([2H])([2H])[2H] ; [2H]C([2H])([2H])C([2H])(Oc1ccc(-c2nc(-c3cccc4c3CC[C@@H]4N(C(=O)OC(C)(C)C)C([2H])([2H])C([2H])([2H])O)no2)cc1C#N)C([2H])([2H])[2H]]

Tert-butylN-[(1S)-4-(5-[3-cyano-4-[(²H₇)propan-2-yloxy]phenyl]-1,2,4-oxadiazol-3-yl)-2,3-dihydro-1H-inden-1-yl]-N-[2-hydroxy(²H₄)ethyl]carbamate: To a solution of 3-cyano-4-[(²H₇)propan-2-yloxy]benzoic acid (562 mg, 2.65 mmol, 1.00 equiv) in DMF (10 mL) were added HOBT (465 mg, 3.44 mmol, 1.30 equiv) and EDC (660 mg, 3.44 mmol, 1.30 equiv). The mixture was stirred for 30 min at room temperature. Then tert-butylN-[2-[(tert-butyldimethylsilyl)oxy](²H₄)ethyl]-N-[(1S)-4-[(E)-N′-hydroxycarbamimidoyl]-2,3-dihydro-1H-inden-1-yl]carbamate (1.2 g, 2.65 mmol, 1.00 equiv) was added. The mixture solution was allowed to stir for 1 h at room temperature. Then the resulting solution was stirred at 80° C. overnight. The reaction was diluted with water (20 mL) and extracted with ethyl acetate (3×30 mL). The organic layers were combined and dried over anhydrous sodium sulfate, filtered and concentrated. The crude product was purified by SiO₂ chromatography, eluted with ethyl acetate/petroleum ether (3:7) to afford 1 g (73%) of tert-butyl N-[(1S)-4-(5-[3-cyano-4-[(²H₇)propan-2-yloxy]phenyl]-1,2,4-oxadiazol-3-yl)-2,3-dihydro-1H-inden-1-yl]-N-[2-hydroxy (²H₄)ethyl]carbamate as light yellow oil. LC-MS: m/z=516 [M+H]⁺.

Step 2

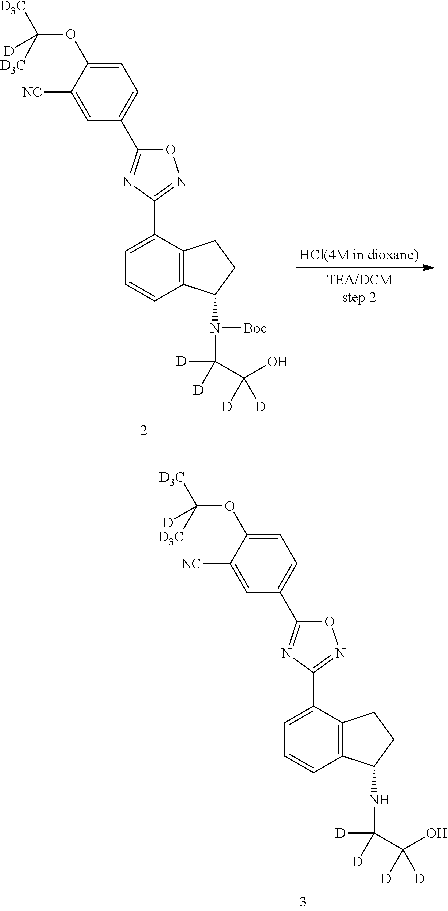 [SMILES: [2H]C([2H])([2H])C([2H])(Oc1ccc(-c2nc(-c3cccc4c3CC[C@@H]4N(C(=O)OC(C)(C)C)C([2H])([2H])C([2H])([2H])O)no2)cc1C#N)C([2H])([2H])[2H] ; [2H]C([2H])([2H])C([2H])(Oc1ccc(-c2nc(-c3cccc4c3CC[C@@H]4NC([2H])([2H])C([2H])([2H])O)no2)cc1C#N)C([2H])([2H])[2H]]

5-[3-[(1S)-1-[[2-hydroxy(²H₇)ethyl]amino]-2,3-dihydro-1H-inden-4-yl]-1,2,4-oxadiazol-5-yl]-2-[(²H₄)propan-2-yloxy]benzonitrile: A solution of tert-butyl N-[(1S)-4-(5-[3-cyano-4-[(²H₄)propan-2-yloxy]phenyl]-1,2,4-oxadiazol-3-yl)-2,3-dihydro-1H-inden-1-yl]-N-[2-hydroxy(²H₇)ethyl]carbamate (600 mg, 1.16 mmol, 1.00 equiv) in hydrogen chloride (4 M in dioxane) (10 mL) was stirred for 6 h at room temperature. The mixture was filtered to obtain 280 mg of white solid. Then the white solid was suspended in DCM (8 mL) and TEA (205 mg, 3.00 equiv) was added. The resulting solution was stirred for 2 h at room temperature. The solution was washed by water (2×20 mL), were dried over anhydrous sodium sulfate, filtered and concentrated. The crude product was purified by Flash-Prep-HPLC with the following conditions: Column, XBridge Shield RP18 OBD Column, Sum, 19*150 mm; mobile phase, Water (0.05% NH₃H₂O) and ACN (45.0% up to 65.0% in 7 min); Detector, UV 254 nm to afford 190 mg (39%) of 5-[3-[(1S)-1-[[2-hydroxy(²H₇)ethyl]amino]-2,3-dihydro-1H-inden-4-yl]-1,2,4-oxadiazol-5-yl]-2-[(²H₄)propan-2-yloxy]benzonitrile as a white solid.

¹H NMR (300 MHz, CDCl₃) δ 8.39-8.38 (m, 1H), 8.34-8.30 (m, 1H), 8.07-8.04 (m, 1H), 7.54-7.51 (m, 1H), 7.39-7.34 (m, 1H), 7.13-7.10 (m, 1H), 4.34-4.30 (m, 1H), 3.48-3.38 (m, 1H), 3.22-3.11 (m, 1H), 2.56-2.45 (m, 1H), 2.30 (s, 2H), 1.96-1.88 (m, 1H). LC-MS: m/z=416 [M+H]⁺.

### Example 6

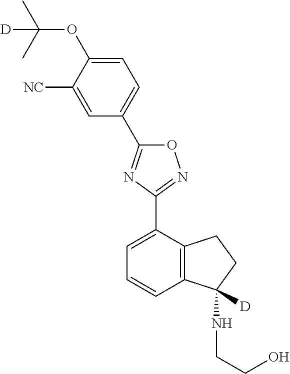 [SMILES: [2H]C(C)(C)Oc1ccc(-c2nc(-c3cccc4c3CC[C@]4([2H])NCCO)no2)cc1C#N]

Step 1

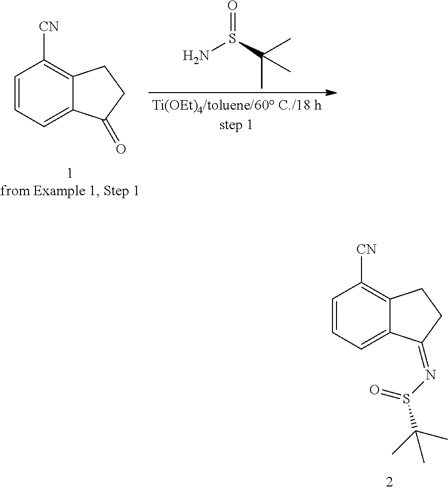

(S)—N-[(1Z)-4-cyano-2,3-dihydro-1H-inden-1-ylidene]-2-methylpropane-2-sulfinamide: To a solution of 1-oxo-2,3-dihydro-1H-indene-4-carbonitrile (5 g, 31.81 mmol, 1.00 equiv) in toluene (50 mL) was added (S)-2-methylpropane-2-sulfinamide (4.6 g, 37.95 mmol, 1.20 equiv), Ti(OEt)₄ (11 g, 1.50 equiv). The resulting solution was stirred for 18 h at 60° C. The reaction progress was monitored by LCMS. The reaction was then quenched by the addition of sat. potassium sodium tartrate (30 mL). The resulting solution was extracted with ethyl acetate (3×50 mL) and the organic layers were combined, washed with brine (2×50 mL), dried over anhydrous sodium sulfate, filtered and concentrated under vacuum to afford 5.5 g (66%) of (S)—N-[(1Z)-4-cyano-2,3-dihydro-1H-inden-1-ylidene]-2-methylpropane-2-sulfinamide as a yellow green solid. LC-MS: m/z=261[M+H]⁺.

Step 2

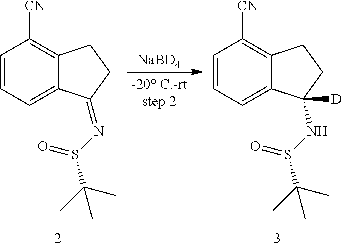

(S)—N-[(1S)-4-cyano-2,3-dihydro(1-4)-1H-inden-1-yl]-2-methylpropane-2-sulfinamide: To a solution of (S)—N-[(1Z)-4-cyano-2,3-dihydro-1H-inden-1-ylidene]-2-methylpropane-2-sulfinamide (5.5 g, 21.13 mmol, 1.00 equiv) in tetrahydrofuran (100 mL) was added NaBD₄ (977 mg, 23.26 mmol, 1.10 equiv), in portions at −20° C. in 20 min. The resulting solution was stirred for 2 h at 25° C. The reaction progress was monitored by LCMS. The reaction was then quenched by the addition of D₂O (10 mL). The resulting solution was extracted with ethyl acetate (3×50 mL) and the organic layers were combined, washed with brine (2×50 mL), dried over anhydrous sodium sulfate, filtered and concentrated under vacuum to afford 4 g (72%) of (S)—N-[(1S)-4-cyano-2,3-dihydro(1-²H)-1H-inden-1-yl]-2-methylpropane-2-sulfinamide as a green solid.

Step 3

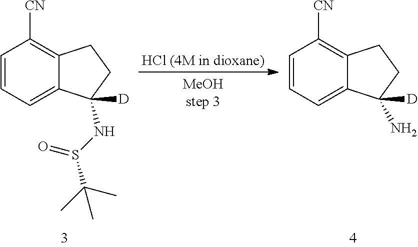

(1S)-1-amino-2,3-dihydro(1-²H)-1H-indene-4-carbonitrile hydrochloride: To a solution of (S)—N-[(1S)-4-cyano-2,3-dihydro(1-²H)-1H-inden-1-yl]-2-methylpropane-2-sulfinamide (4 g, 15.19 mmol, 1.00 equiv) in methanol (40 mL) was added hydrogen chloride (4 M in dioxane) (12 mL, 3.00 equiv). The resulting solution was stirred for 2 h at 25° C. The reaction progress was monitored by LCMS. The resulting mixture was concentrated under vacuum. The residue was dissolved in MeCN (50 mL), refluxed for 30 min and then cooled to room temperature. The solids were collected by filtration to afford 2.8 g (94%) of (1S)-1-amino-2,3-dihydro(1-²H)-1H-indene-4-carbonitrile hydrochloride as a yellow green solid. LC-MS: m/z=160[M+H]⁺.

Step 4

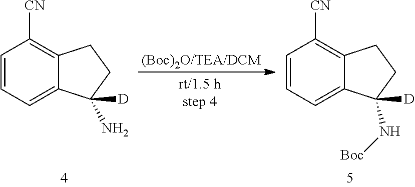

Tert-butyl N-[(1S)-4-cyano-2,3-dihydro(1-²H)-1H-inden-1-yl]carbamate: To a solution of (1S)-1-amino-2,3-dihydro (1-²H)-1H-indene-4-carbonitrile hydrochloride (2.8 g, 14.31 mmol, 1.00 equiv) in dichloromethane (50 mL) were added TEA (3.6 g, 35.58 mmol, 2.50 equiv) and (Boc)₂O (3.4 g, 15.58 mmol, 1.10 equiv). The resulting solution was stirred for 1.5 h at 25° C. The reaction progress was monitored by LCMS. The resulting solution was diluted with DCM (50 mL), washed with brine (2×50 mL), dried over anhydrous sodium sulfate, filtered and concentrated under vacuum. The residue was purified by a silica gel column eluting with ethyl acetate/petroleum ether (1:20) to afford 2.2 g (59%) of tert-butyl N-[(1S)-4-cyano-2,3-dihydro(1-²H)-1H-inden-1-yl]carbamate as a white solid. LC-MS: m/z=260[M+H]⁺.

Step 5

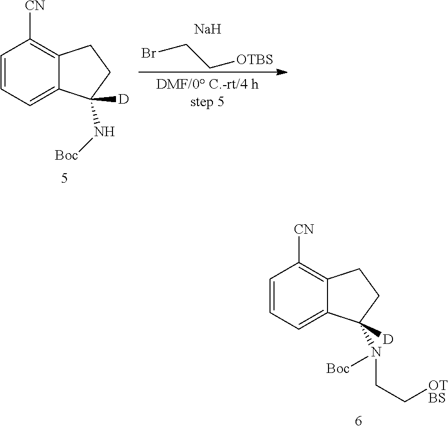

Tert-butyl N-[2-[(tert-butyldimethylsilyl)oxy]ethyl]-N-[(1S)-4-cyano-2,3-dihydro(1-²H)-1H-inden-1-yl]carbamate: To a solution of tert-butyl N-[(1S)-4-cyano-2,3-dihydro(1-²H)-1H-inden-1-yl]carbamate (1.7 g, 6.56 mmol, 1.00 equiv) in N,N-dimethylformamide (20 mL) at 0° C. was added sodium hydride (530 mg, 13.25 mmol, 2.00 equiv) in portions. The resulting solution was stirred at room temperature for 2 h. Then (2-bromoethoxy)(tert-butyl)dimethylsilane (3.14 g, 13.13 mmol, 2.00 equiv) was added. The resulting solution was stirred for 4 h at room temperature. The reaction was then quenched by the addition of water/ice, extracted with ethyl acetate (3×100 mL) and the organic layers were combined. The resulting mixture was washed with brine (2×100 mL), dried over anhydrous sodium sulfate, filtered and concentrated under vacuum. The residue was purified by a silica gel column with ethyl acetate/petroleum ether (1:20) to afford 1.27 g (46%) of tert-butyl N-[2-[(tert-butyldimethylsilyl)oxy]ethyl]-N-[(1S)-4-cyano-2,3-dihydro(1-²H)-1H-inden-1-yl]carbamate as light brown oil. LC-MS: m/z=418[M+H]⁺.

Step 6

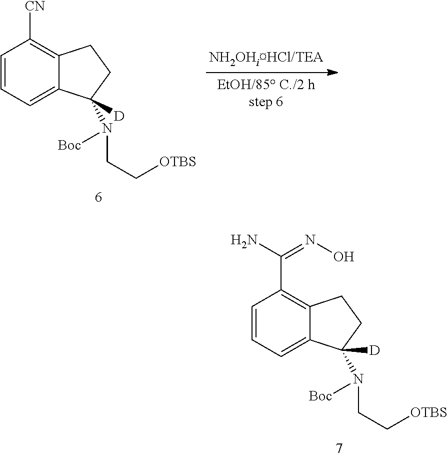 [SMILES: [2H][C@]1(N(CCO[Si](C)(C)C(C)(C)C)C(=O)OC(C)(C)C)CCc2c(C#N)cccc21 ; [2H][C@]1(N(CCO[Si](C)(C)C(C)(C)C)C(=O)OC(C)(C)C)CCc2c(/C(N)=N\O)cccc21]

Tert-butyl N-[2-[(tert-butyldimethylsilyl)oxy]ethyl]-N-[(1S)-4-[(Z)—N\_ydroxycarbamimidoyl]-2,3-dihydro(1-²H)-1H-inden-1-yl]carbamate: To a solution of tert-butyl N-[2-[(tert-butyldimethylsilyl)oxy]ethyl]-N-[(1S)-4-cyano-2,3-dihydro(1-²H)-1H-inden-1-yl]carbamate (1.27 g, 3.04 mmol, 1.00 equiv) in ethanol (20 mL) were added NH₂OHHCl (630 mg, 9.13 mmol, 3.00 equiv), TEA (920 mg, 9.09 mmol, 3.00 equiv). The resulting solution was stirred for 2 h at 85° C. and then concentrated under vacuum. The resulting solution was diluted with water (40 mL), extracted with dichloromethane (3×50 mL), dried over anhydrous sodium sulfate, filtered and concentrated under vacuum to afford 1 g (73%) of tert-butyl N-[2-[(tert-butyldimethylsilyl)oxy]ethyl]-N-[(1S)-4-[(Z)—N′-hydroxycarbamimidoyl]-2,3-dihydro(1-²H)-1H-inden-1-yl]carbamate as light yellow oil. LC-MS: m/z=451 [M+H]⁺.

Step 7

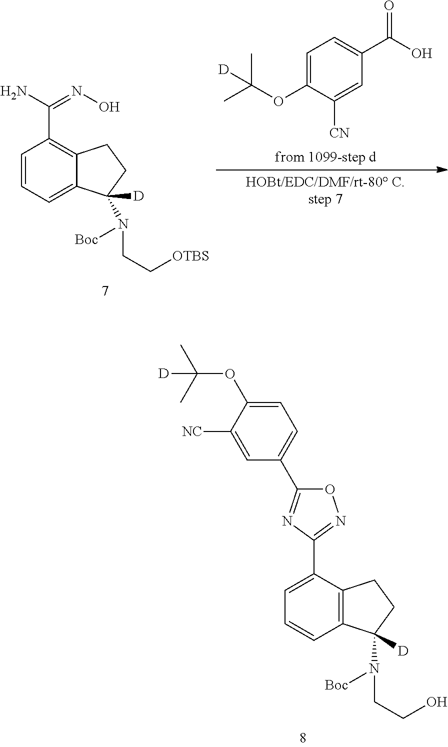 [SMILES: [2H][C@]1(N(CCO[Si](C)(C)C(C)(C)C)C(=O)OC(C)(C)C)CCc2c(/C(N)=N\O)cccc21 ; [2H]C(C)(C)Oc1ccc(C(=O)O)cc1C#N ; [2H]C(C)(C)Oc1ccc(-c2nc(-c3cccc4c3CC[C@]4([2H])N(CCO)C(=O)OC(C)(C)C)no2)cc1C#N]

Tert-butyl N-[(1S)-4-(5-[3-cyano-4-[(2-²H)propan-2-yloxy]phenyl]-1,2,4-oxadiazol-3-yl)-2,3-dihydro(1-²H)-1H-inden-1-yl]-N-(2-hydroxyethyl)carbamate: To a solution of 3-cyano-4-[(2-²H)propan-2-yloxy]benzoic acid (460 mg, 2.23 mmol, 1.00 equiv) in N,N-dimethylformamide (10 mL) was added HOBT (390 mg, 2.89 mmol, 1.30 equiv), EDC(HCl) (560 mg, 2.92 mmol, 1.31 equiv). The resulting solution was stirred at room temperature for 0.5 h. Then tert-butyl N-[2-[(tert-butyldimethylsilyl)oxy]ethyl]-N-[(1S)-4-[(Z)—N′-hydroxycarbamimidoyl]-2,3-dihydro(1-²H)-1H-inden-1-yl]carbamate (1 g, 2.22 mmol, 1.00 equiv) was added. The resulting solution was stirred at room temperature for 1 h, then stirred overnight at 80° C. The reaction mixture was cooled and diluted with water. The resulting solution was extracted with ethyl acetate (3×50 mL). The organic layers were combined, dried over anhydrous sodium sulfate, filtered and concentrated under vacuum. The residue was purified by a silica gel column eluting with ethyl acetate/petroleum ether (1:5-1:3) to afford 0.9 g (80%) of tert-butyl N-[(1S)-4-(5-[3-cyano-4-[(2-²H)propan-2-yloxy]phenyl]-1,2,4-oxadiazol-3-yl)-2,3-dihydro(1-²H)-1H-inden-1-yl]-N-(2-hydroxyethyl)carbamate as light brown oil. LC-MS: m/z=507[M+H]⁺.

Step 8

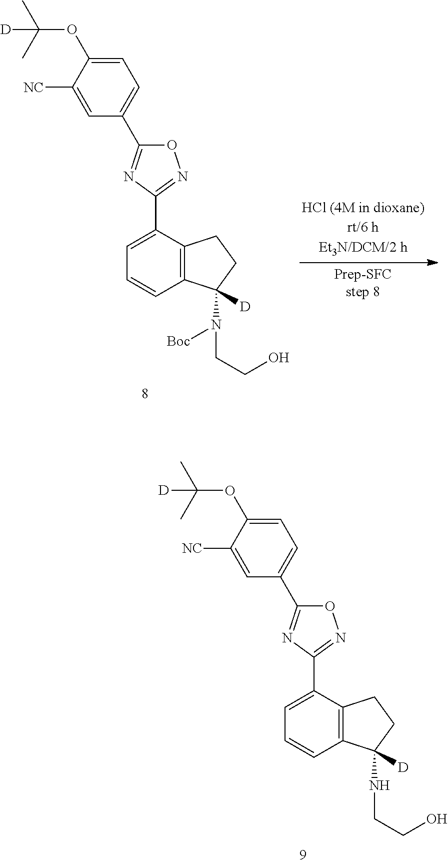 [SMILES: [2H]C(C)(C)Oc1ccc(-c2nc(-c3cccc4c3CC[C@]4([2H])N(CCO)C(=O)OC(C)(C)C)no2)cc1C#N ; [2H]C(C)(C)Oc1ccc(-c2nc(-c3cccc4c3CC[C@]4([2H])NCCO)no2)cc1C#N]

5-[3-[(1S)-1-[(2-hydroxyethyl)amino]-2,3-dihydro(1-²H)-1H-inden-4-yl]-1,2,4-oxadiazol-5-yl]-2-[(2-²H)propan-2-yloxy]benzonitrile: To a solution of tert-butyl N-[(1S)-4-(5-[3-cyano-4-[(2-²H)propan-2-yloxy]phenyl]-1,2,4-oxadiazol-3-yl)-2,3-dihydro(1-²H)-1H-inden-1-yl]-N-(2-hydroxyethyl) carbamate (500 mg, 0.99 mmol, 1.00 equiv) was added hydrogen chloride (4 M in dioxane) (10 mL). The resulting solution was stirred at room temperature for 6 h. The solid was collected by filtration and suspended in DCM (10 mL). Then triethylamine (300 mg, 2.97 mmol, 3.00 equiv) was added and the mixture was stirred for 2 h at room temperature. The resulting solution was washed by water (2×20 mL), dried over anhydrous sodium sulfate, filtered and concentrated under vacuum. The crude product was purified by Prep-SFC with the following conditions: Column: Phenomenex Lux 5u Cellulose-4, AXIA Packed, 250*21.2 mm, 5 um; Mobile Phase A: CO₂:50, Mobile Phase B: MeOH (0.2% DEA):50; Flow rate: 50 mL/min; 220 nm; RT: 6.12 to afford 139.1 mg (35%) of 5-[3-[(1S)-1-[(2-hydroxyethyl)amino]-2,3-dihydro(1-²H)-1H-inden-4-yl]-1,2,4-oxadiazol-5-yl]-2-[(2-²H)propan-2-yloxy]benzonitrile as a white solid. 1H NMR (300 MHz, Chloroform-d) δ 8.44-8.27 (m, 2H), 8.06 (m, 1H), 7.52 (m, 1H), 7.37 (t, J=7.6 Hz, 1H), 7.11 (d, J=8.9 Hz, 1H), 3.69 (m, 2H), 3.44 (m, 1H), 3.26-3.09 (m, 1H), 2.91 (m, 2H), 2.50 (m, 1H), 2.19 (brs, 2H), 1.90 (m, 1H), 1.47 (s, 6H). LC-MS: m/z=406[M+H]⁺.

The following compounds can generally be made using the methods described above. It is expected that these compounds when made will have activity similar to those described in the examples above.

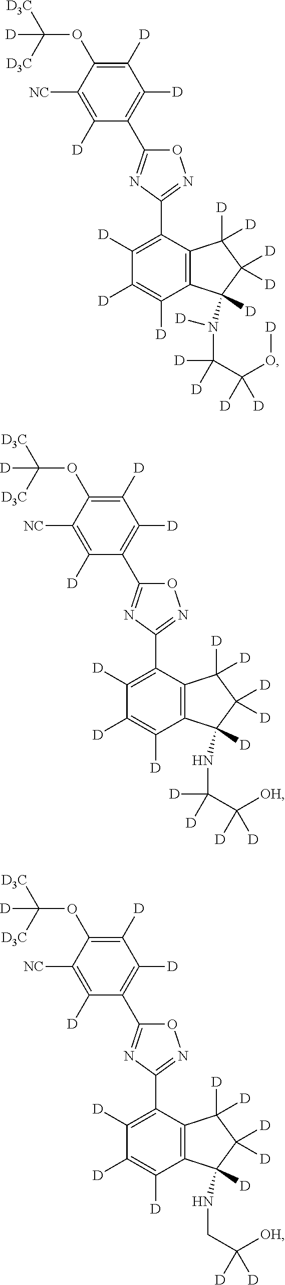

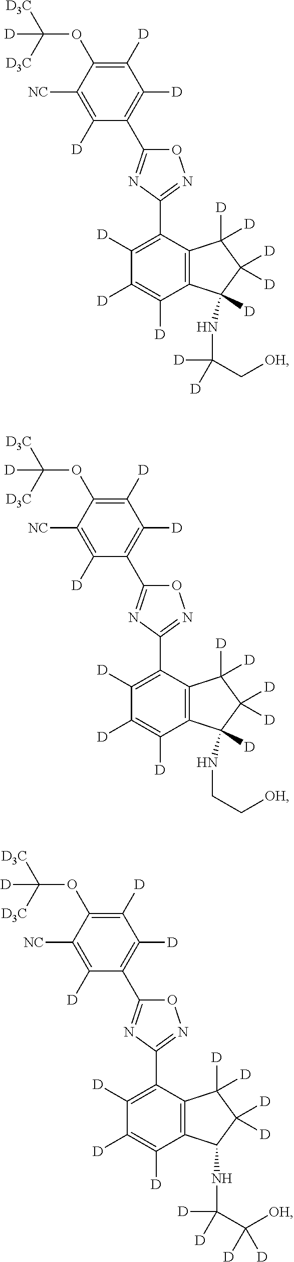

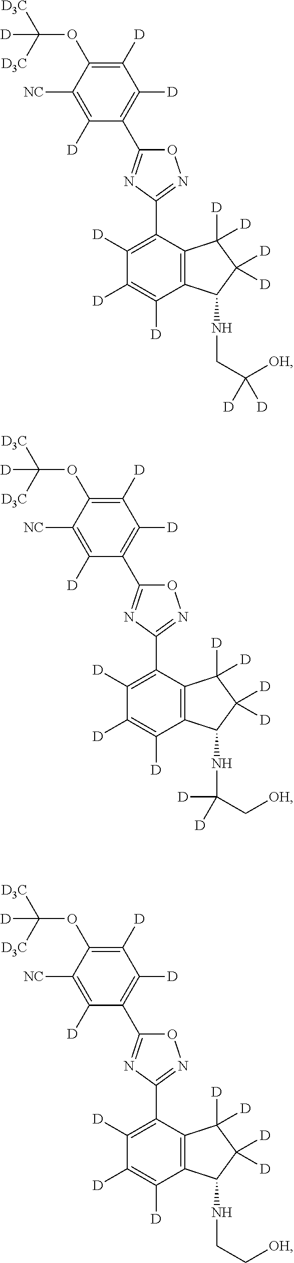 [SMILES: [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@H](NCC([2H])([2H])O)C([2H])([2H])C2([2H])[2H] ; [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@H](NC([2H])([2H])CO)C([2H])([2H])C2([2H])[2H] ; [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@H](NCCO)C([2H])([2H])C2([2H])[2H]]

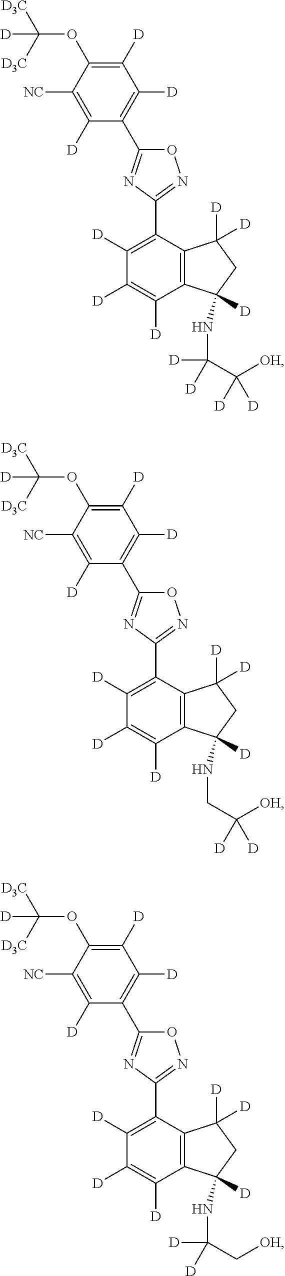 [SMILES: [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@]([2H])(NC([2H])([2H])C([2H])([2H])O)CC2([2H])[2H] ; [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@]([2H])(NCC([2H])([2H])O)CC2([2H])[2H] ; [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@]([2H])(NC([2H])([2H])CO)CC2([2H])[2H]]

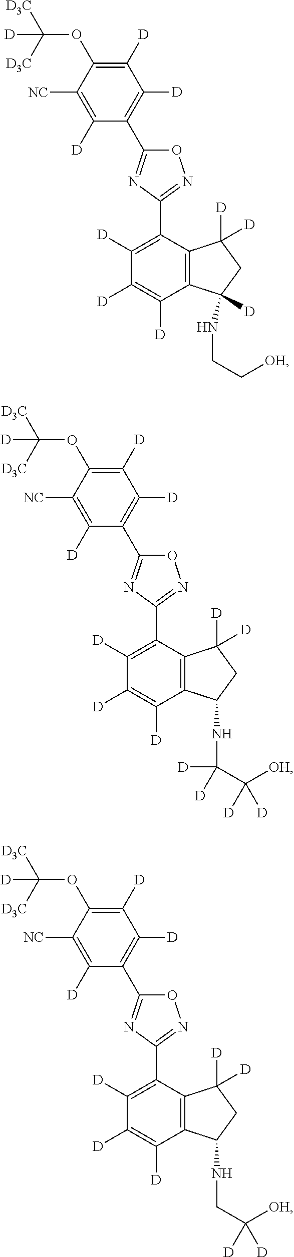 [SMILES: [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@]([2H])(NCCO)CC2([2H])[2H] ; [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@H](NC([2H])([2H])C([2H])([2H])O)CC2([2H])[2H] ; [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@H](NCC([2H])([2H])O)CC2([2H])[2H]]

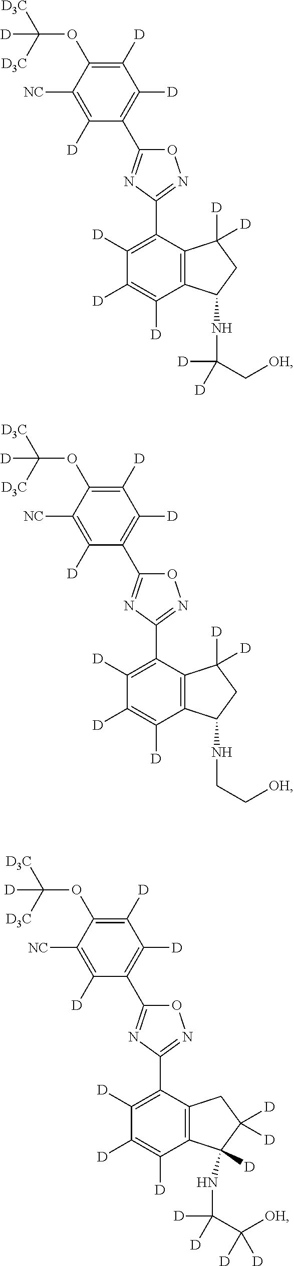 [SMILES: [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@H](NC([2H])([2H])CO)CC2([2H])[2H] ; [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@H](NCCO)CC2([2H])[2H] ; [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@]([2H])(NC([2H])([2H])C([2H])([2H])O)C([2H])([2H])C2]

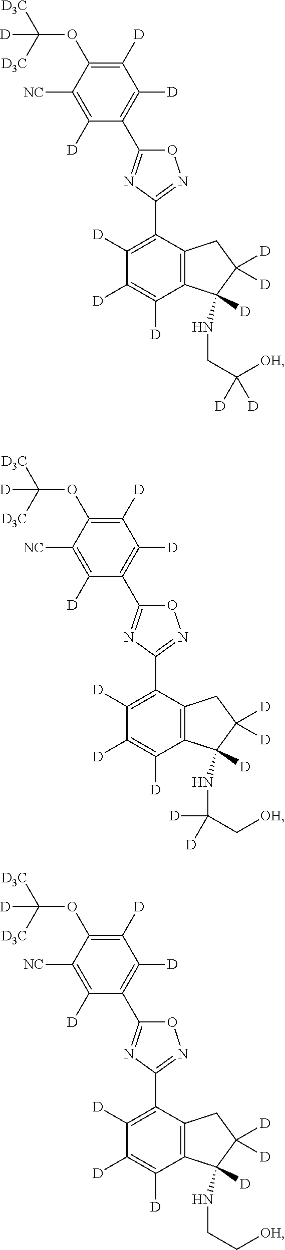 [SMILES: [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@]([2H])(NCC([2H])([2H])O)C([2H])([2H])C2 ; [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@]([2H])(NC([2H])([2H])CO)C([2H])([2H])C2 ; [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@]([2H])(NCCO)C([2H])([2H])C2]

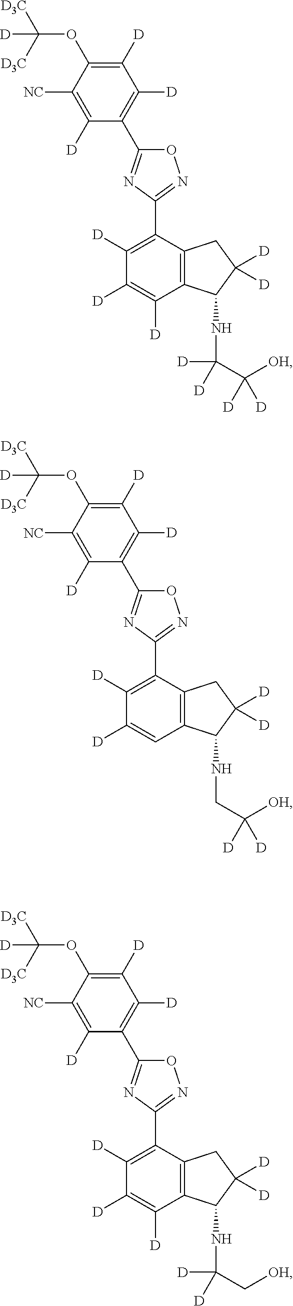 [SMILES: [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@H](NC([2H])([2H])C([2H])([2H])O)C([2H])([2H])C2 ; [2H]c1cc2c(c(-c3noc(-c4c([2H])c([2H])c(OC([2H])(C([2H])([2H])[2H])C([2H])([2H])[2H])c(C#N)c4[2H])n3)c1[2H])CC([2H])([2H])[C@@H]2NCC([2H])([2H])O ; [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@H](NC([2H])([2H])CO)C([2H])([2H])C2]

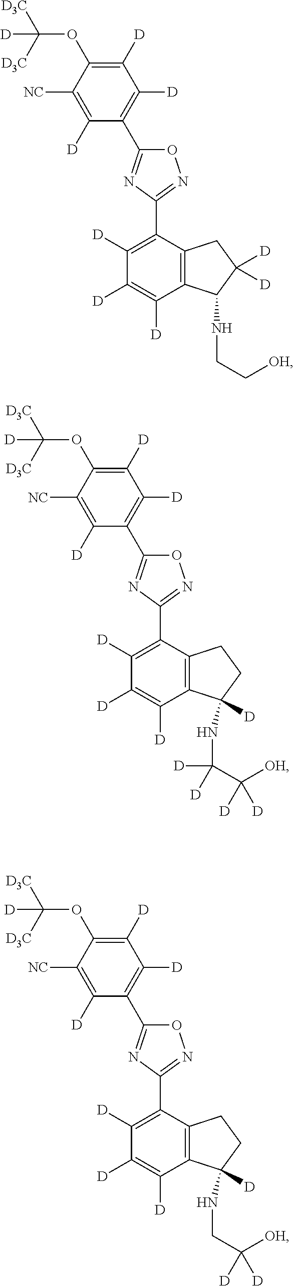 [SMILES: [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@H](NCCO)C([2H])([2H])C2 ; [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@]([2H])(NC([2H])([2H])C([2H])([2H])O)CC2 ; [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@]([2H])(NCC([2H])([2H])O)CC2]

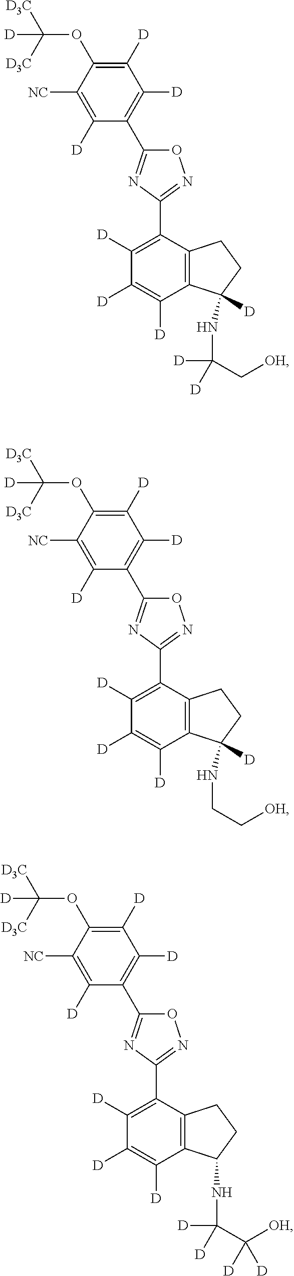 [SMILES: [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@]([2H])(NC([2H])([2H])CO)CC2 ; [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@]([2H])(NCCO)CC2 ; [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@H](NC([2H])([2H])C([2H])([2H])O)CC2]

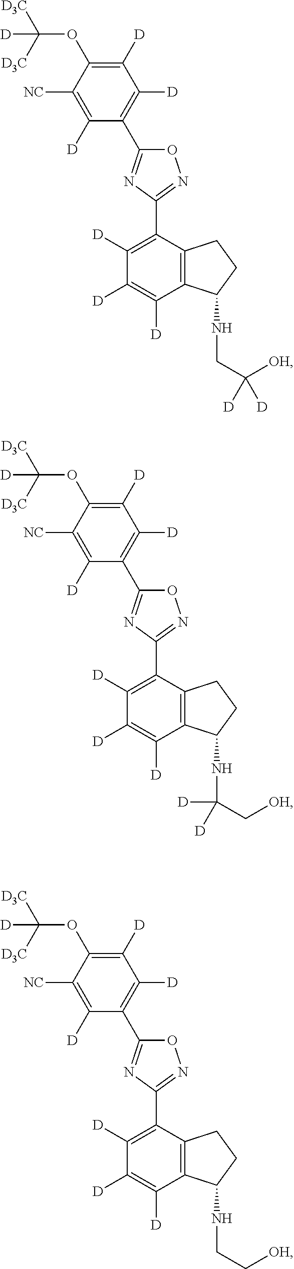 [SMILES: [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@H](NCC([2H])([2H])O)CC2 ; [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@H](NC([2H])([2H])CO)CC2 ; [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@H](NCCO)CC2]

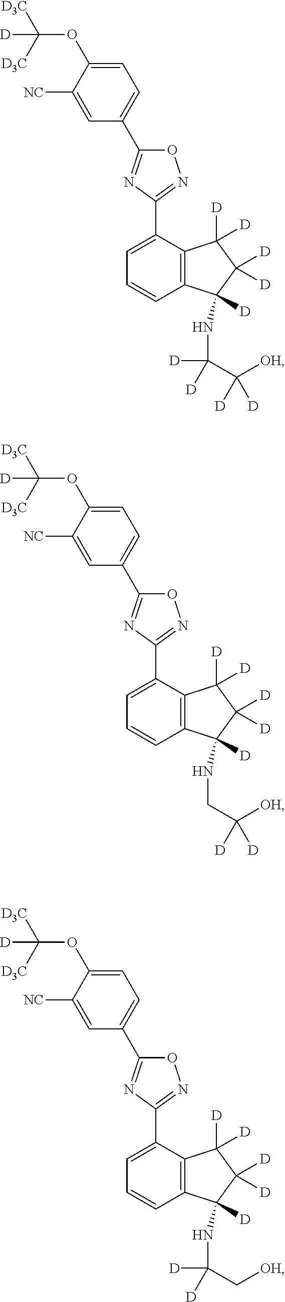 [SMILES: [2H]C([2H])([2H])C([2H])(Oc1ccc(-c2nc(-c3cccc4c3C([2H])([2H])C([2H])([2H])[C@]4([2H])NC([2H])([2H])C([2H])([2H])O)no2)cc1C#N)C([2H])([2H])[2H] ; [2H]C([2H])(O)CN[C@]1([2H])c2cccc(-c3noc(-c4ccc(OC([2H])(C([2H])([2H])[2H])C([2H])([2H])[2H])c(C#N)c4)n3)c2C([2H])([2H])C1([2H])[2H] ; [2H]C([2H])(CO)N[C@]1([2H])c2cccc(-c3noc(-c4ccc(OC([2H])(C([2H])([2H])[2H])C([2H])([2H])[2H])c(C#N)c4)n3)c2C([2H])([2H])C1([2H])[2H]]

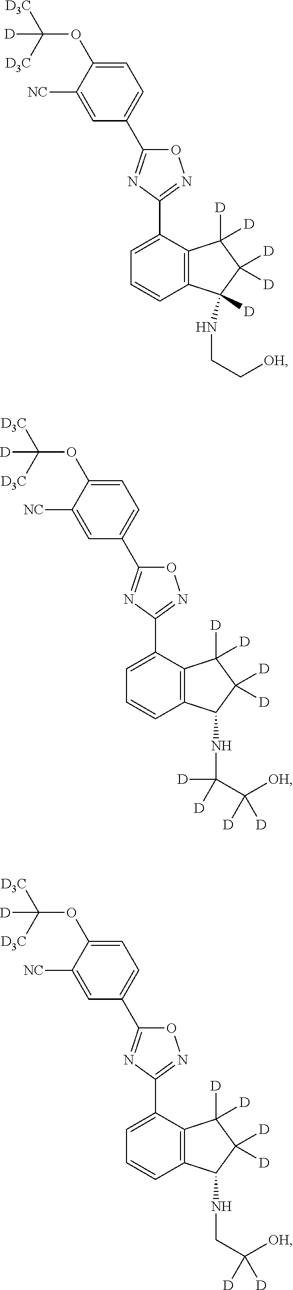 [SMILES: [2H]C([2H])([2H])C([2H])(Oc1ccc(-c2nc(-c3cccc4c3C([2H])([2H])C([2H])([2H])[C@]4([2H])NCCO)no2)cc1C#N)C([2H])([2H])[2H] ; [2H]C([2H])([2H])C([2H])(Oc1ccc(-c2nc(-c3cccc4c3C([2H])([2H])C([2H])([2H])[C@@H]4NC([2H])([2H])C([2H])([2H])O)no2)cc1C#N)C([2H])([2H])[2H] ; [2H]C([2H])(O)CN[C@@H]1c2cccc(-c3noc(-c4ccc(OC([2H])(C([2H])([2H])[2H])C([2H])([2H])[2H])c(C#N)c4)n3)c2C([2H])([2H])C1([2H])[2H]]

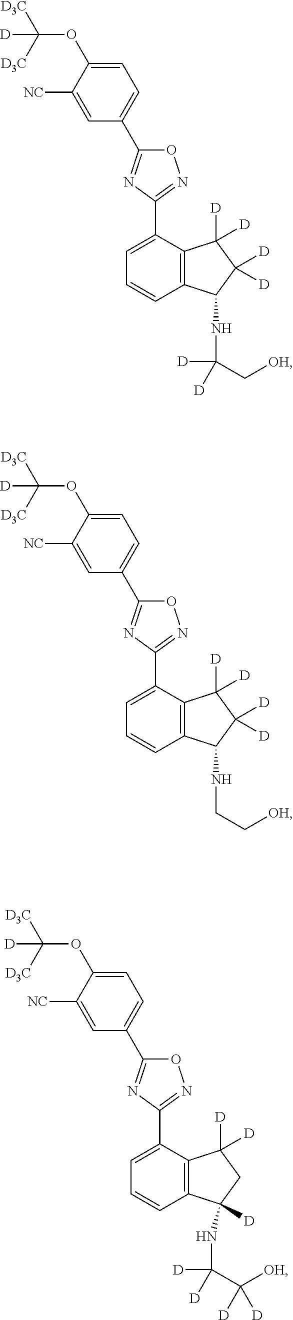 [SMILES: [2H]C([2H])(CO)N[C@@H]1c2cccc(-c3noc(-c4ccc(OC([2H])(C([2H])([2H])[2H])C([2H])([2H])[2H])c(C#N)c4)n3)c2C([2H])([2H])C1([2H])[2H] ; [2H]C([2H])([2H])C([2H])(Oc1ccc(-c2nc(-c3cccc4c3C([2H])([2H])C([2H])([2H])[C@@H]4NCCO)no2)cc1C#N)C([2H])([2H])[2H] ; [2H]C1([2H])C[C@]([2H])(NC([2H])([2H])C([2H])([2H])O)c2cccc(-c3noc(-c4ccc(OC([2H])(C([2H])([2H])[2H])C([2H])([2H])[2H])c(C#N)c4)n3)c21]

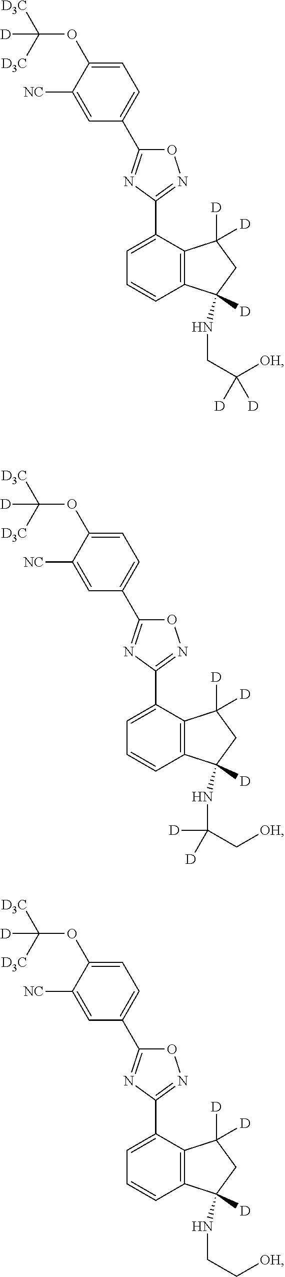 [SMILES: [2H]C([2H])(O)CN[C@@]1([2H])CC([2H])([2H])c2c(-c3noc(-c4ccc(OC([2H])(C([2H])([2H])[2H])C([2H])([2H])[2H])c(C#N)c4)n3)cccc21 ; [2H]C([2H])(CO)N[C@@]1([2H])CC([2H])([2H])c2c(-c3noc(-c4ccc(OC([2H])(C([2H])([2H])[2H])C([2H])([2H])[2H])c(C#N)c4)n3)cccc21 ; [2H]C1([2H])C[C@]([2H])(NCCO)c2cccc(-c3noc(-c4ccc(OC([2H])(C([2H])([2H])[2H])C([2H])([2H])[2H])c(C#N)c4)n3)c21]

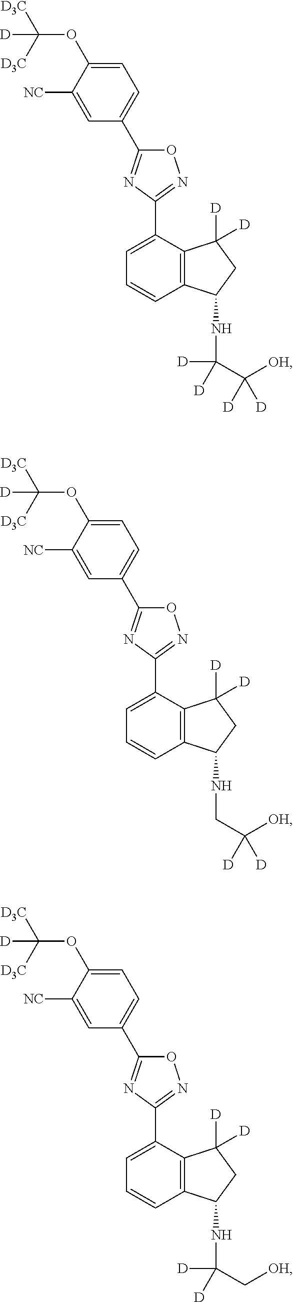 [SMILES: [2H]C1([2H])C[C@H](NC([2H])([2H])C([2H])([2H])O)c2cccc(-c3noc(-c4ccc(OC([2H])(C([2H])([2H])[2H])C([2H])([2H])[2H])c(C#N)c4)n3)c21 ; [2H]C([2H])(O)CN[C@H]1CC([2H])([2H])c2c(-c3noc(-c4ccc(OC([2H])(C([2H])([2H])[2H])C([2H])([2H])[2H])c(C#N)c4)n3)cccc21 ; [2H]C([2H])(CO)N[C@H]1CC([2H])([2H])c2c(-c3noc(-c4ccc(OC([2H])(C([2H])([2H])[2H])C([2H])([2H])[2H])c(C#N)c4)n3)cccc21]

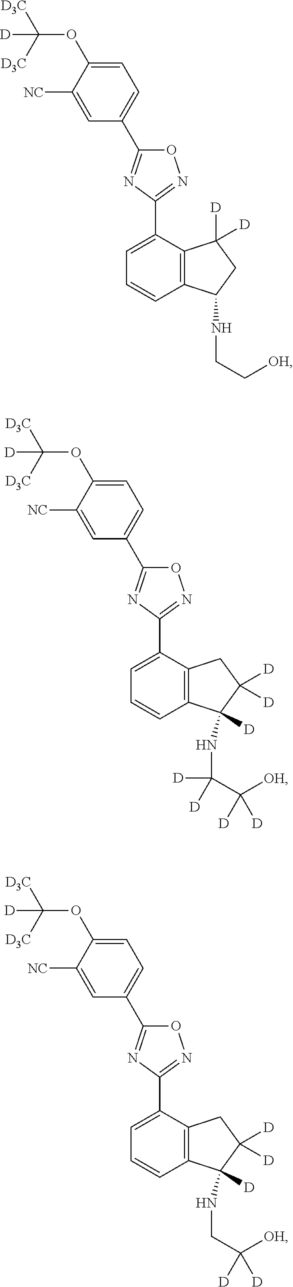 [SMILES: [2H]C1([2H])C[C@H](NCCO)c2cccc(-c3noc(-c4ccc(OC([2H])(C([2H])([2H])[2H])C([2H])([2H])[2H])c(C#N)c4)n3)c21 ; [2H]C([2H])([2H])C([2H])(Oc1ccc(-c2nc(-c3cccc4c3CC([2H])([2H])[C@]4([2H])NC([2H])([2H])C([2H])([2H])O)no2)cc1C#N)C([2H])([2H])[2H] ; [2H]C([2H])(O)CN[C@]1([2H])c2cccc(-c3noc(-c4ccc(OC([2H])(C([2H])([2H])[2H])C([2H])([2H])[2H])c(C#N)c4)n3)c2CC1([2H])[2H]]

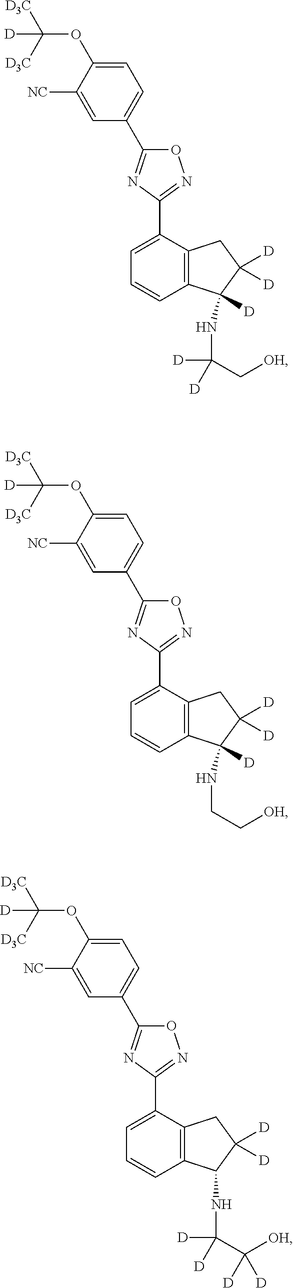 [SMILES: [2H]C([2H])(CO)N[C@]1([2H])c2cccc(-c3noc(-c4ccc(OC([2H])(C([2H])([2H])[2H])C([2H])([2H])[2H])c(C#N)c4)n3)c2CC1([2H])[2H] ; [2H]C([2H])([2H])C([2H])(Oc1ccc(-c2nc(-c3cccc4c3CC([2H])([2H])[C@]4([2H])NCCO)no2)cc1C#N)C([2H])([2H])[2H] ; [2H]C1([2H])Cc2c(-c3noc(-c4ccc(OC([2H])(C([2H])([2H])[2H])C([2H])([2H])[2H])c(C#N)c4)n3)cccc2[C@H]1NC([2H])([2H])C([2H])([2H])O]

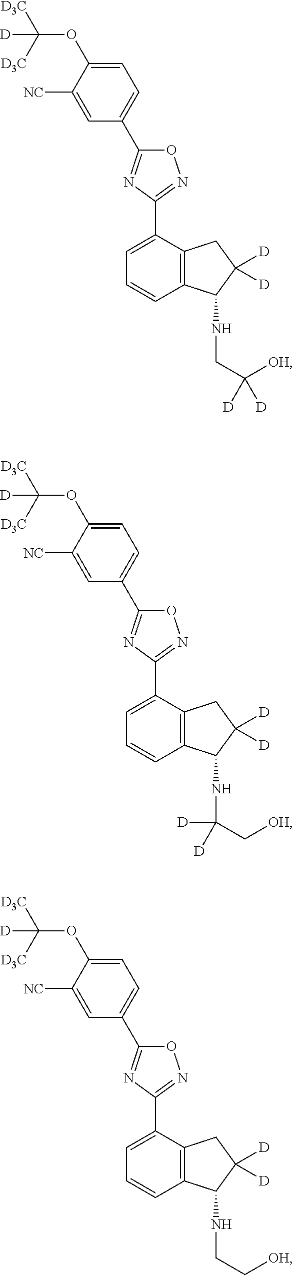 [SMILES: [2H]C([2H])(O)CN[C@@H]1c2cccc(-c3noc(-c4ccc(OC([2H])(C([2H])([2H])[2H])C([2H])([2H])[2H])c(C#N)c4)n3)c2CC1([2H])[2H] ; [2H]C([2H])(CO)N[C@@H]1c2cccc(-c3noc(-c4ccc(OC([2H])(C([2H])([2H])[2H])C([2H])([2H])[2H])c(C#N)c4)n3)c2CC1([2H])[2H] ; [2H]C1([2H])Cc2c(-c3noc(-c4ccc(OC([2H])(C([2H])([2H])[2H])C([2H])([2H])[2H])c(C#N)c4)n3)cccc2[C@H]1NCCO]

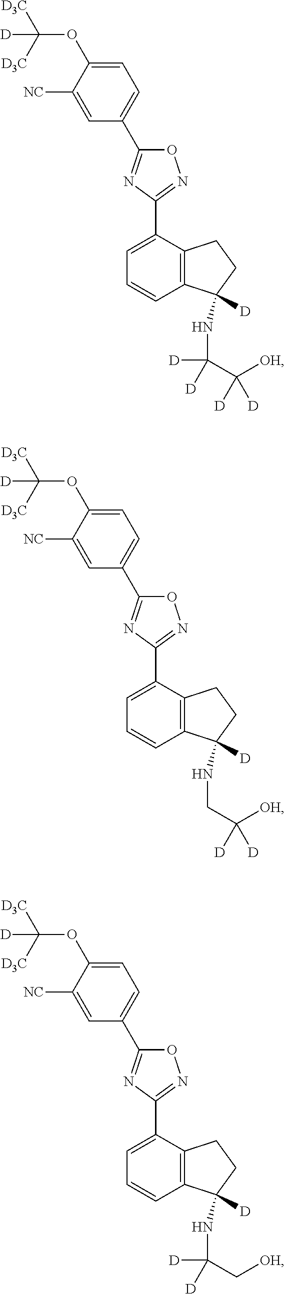 [SMILES: [2H]C([2H])([2H])C([2H])(Oc1ccc(-c2nc(-c3cccc4c3CC[C@]4([2H])NC([2H])([2H])C([2H])([2H])O)no2)cc1C#N)C([2H])([2H])[2H] ; [2H]C([2H])(O)CN[C@@]1([2H])CCc2c(-c3noc(-c4ccc(OC([2H])(C([2H])([2H])[2H])C([2H])([2H])[2H])c(C#N)c4)n3)cccc21 ; [2H]C([2H])(CO)N[C@@]1([2H])CCc2c(-c3noc(-c4ccc(OC([2H])(C([2H])([2H])[2H])C([2H])([2H])[2H])c(C#N)c4)n3)cccc21]

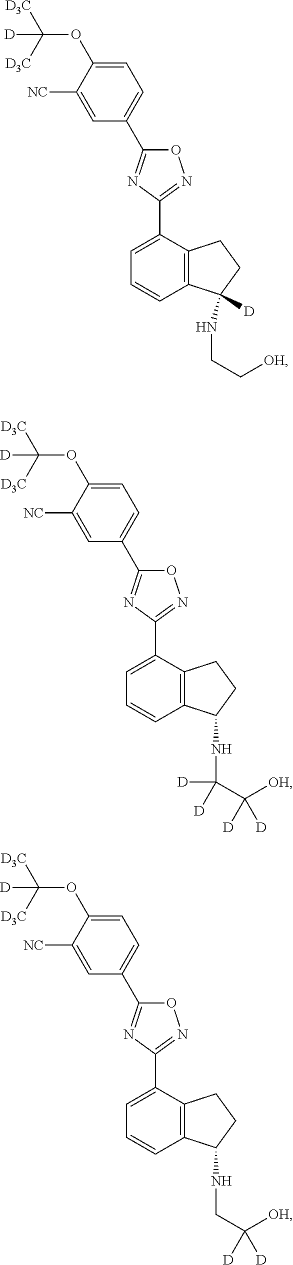 [SMILES: [2H]C([2H])([2H])C([2H])(Oc1ccc(-c2nc(-c3cccc4c3CC[C@]4([2H])NCCO)no2)cc1C#N)C([2H])([2H])[2H] ; [2H]C([2H])([2H])C([2H])(Oc1ccc(-c2nc(-c3cccc4c3CC[C@@H]4NC([2H])([2H])C([2H])([2H])O)no2)cc1C#N)C([2H])([2H])[2H] ; [2H]C([2H])(O)CN[C@H]1CCc2c(-c3noc(-c4ccc(OC([2H])(C([2H])([2H])[2H])C([2H])([2H])[2H])c(C#N)c4)n3)cccc21]

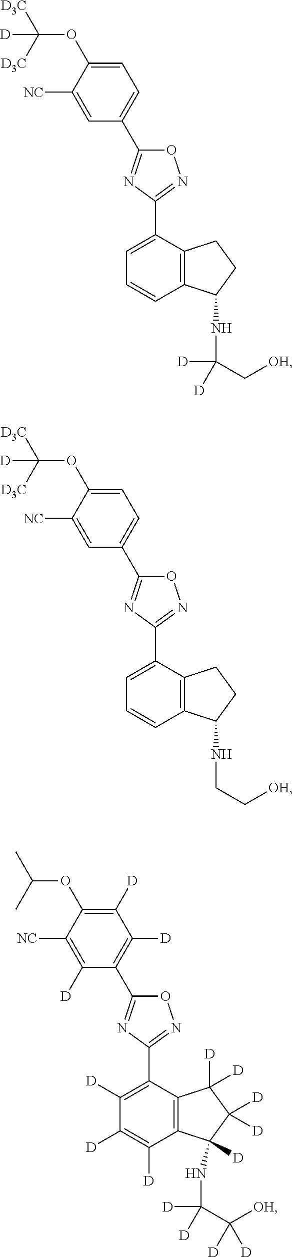 [SMILES: [2H]C([2H])(CO)N[C@H]1CCc2c(-c3noc(-c4ccc(OC([2H])(C([2H])([2H])[2H])C([2H])([2H])[2H])c(C#N)c4)n3)cccc21 ; [2H]C([2H])([2H])C([2H])(Oc1ccc(-c2nc(-c3cccc4c3CC[C@@H]4NCCO)no2)cc1C#N)C([2H])([2H])[2H] ; [2H]c1c([2H])c(-c2noc(-c3c([2H])c([2H])c(OC(C)C)c(C#N)c3[2H])n2)c2c(c1[2H])[C@@]([2H])(NC([2H])([2H])C([2H])([2H])O)C([2H])([2H])C2([2H])[2H]]

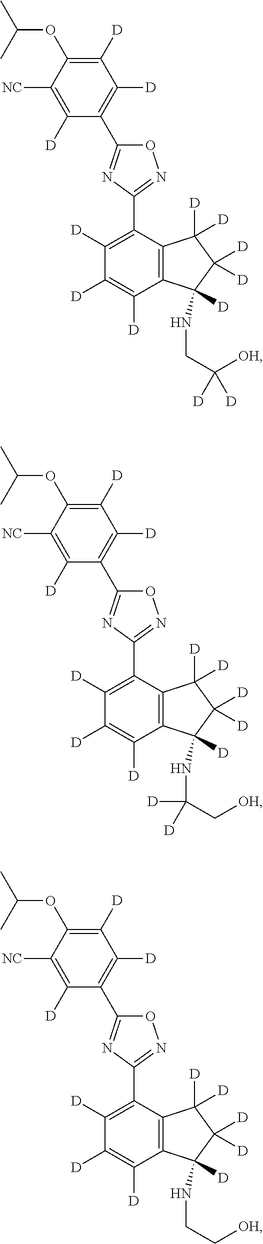 [SMILES: [2H]c1c([2H])c(-c2noc(-c3c([2H])c([2H])c(OC(C)C)c(C#N)c3[2H])n2)c2c(c1[2H])[C@@]([2H])(NCC([2H])([2H])O)C([2H])([2H])C2([2H])[2H] ; [2H]c1c([2H])c(-c2noc(-c3c([2H])c([2H])c(OC(C)C)c(C#N)c3[2H])n2)c2c(c1[2H])[C@@]([2H])(NC([2H])([2H])CO)C([2H])([2H])C2([2H])[2H] ; [2H]c1c([2H])c(-c2noc(-c3c([2H])c([2H])c(OC(C)C)c(C#N)c3[2H])n2)c2c(c1[2H])[C@@]([2H])(NCCO)C([2H])([2H])C2([2H])[2H]]

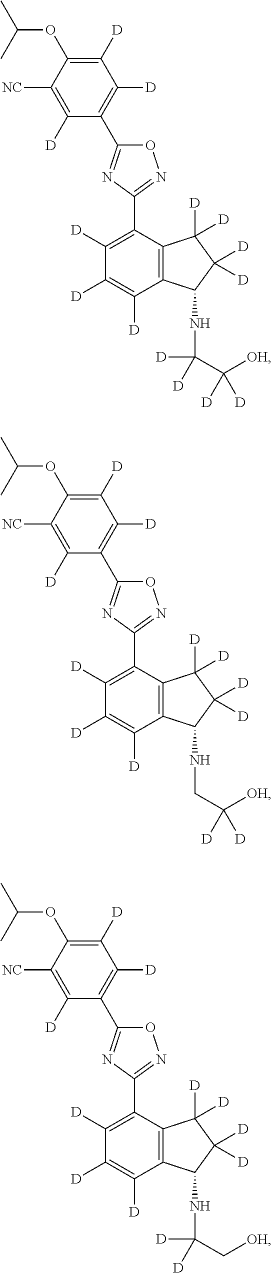 [SMILES: [2H]c1c([2H])c(-c2noc(-c3c([2H])c([2H])c(OC(C)C)c(C#N)c3[2H])n2)c2c(c1[2H])[C@@H](NC([2H])([2H])C([2H])([2H])O)C([2H])([2H])C2([2H])[2H] ; [2H]c1c([2H])c(-c2noc(-c3c([2H])c([2H])c(OC(C)C)c(C#N)c3[2H])n2)c2c(c1[2H])[C@@H](NCC([2H])([2H])O)C([2H])([2H])C2([2H])[2H] ; [2H]c1c([2H])c(-c2noc(-c3c([2H])c([2H])c(OC(C)C)c(C#N)c3[2H])n2)c2c(c1[2H])[C@@H](NC([2H])([2H])CO)C([2H])([2H])C2([2H])[2H]]

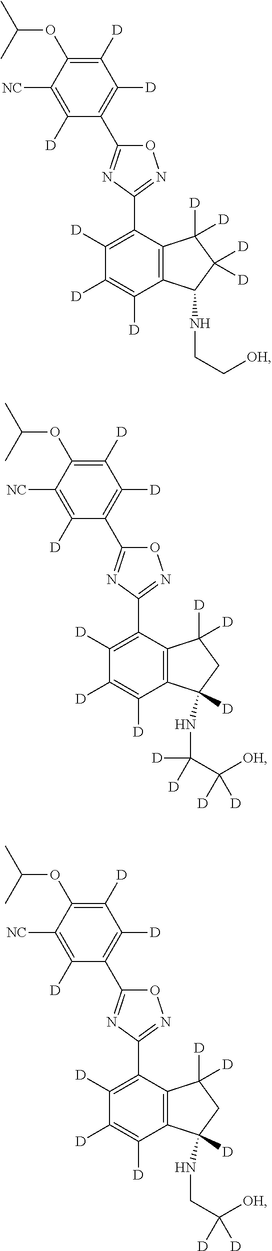 [SMILES: [2H]c1c([2H])c(-c2noc(-c3c([2H])c([2H])c(OC(C)C)c(C#N)c3[2H])n2)c2c(c1[2H])[C@@H](NCCO)C([2H])([2H])C2([2H])[2H] ; [2H]c1c([2H])c(-c2noc(-c3c([2H])c([2H])c(OC(C)C)c(C#N)c3[2H])n2)c2c(c1[2H])[C@@]([2H])(NC([2H])([2H])C([2H])([2H])O)CC2([2H])[2H] ; [2H]c1c([2H])c(-c2noc(-c3c([2H])c([2H])c(OC(C)C)c(C#N)c3[2H])n2)c2c(c1[2H])[C@@]([2H])(NCC([2H])([2H])O)CC2([2H])[2H]]

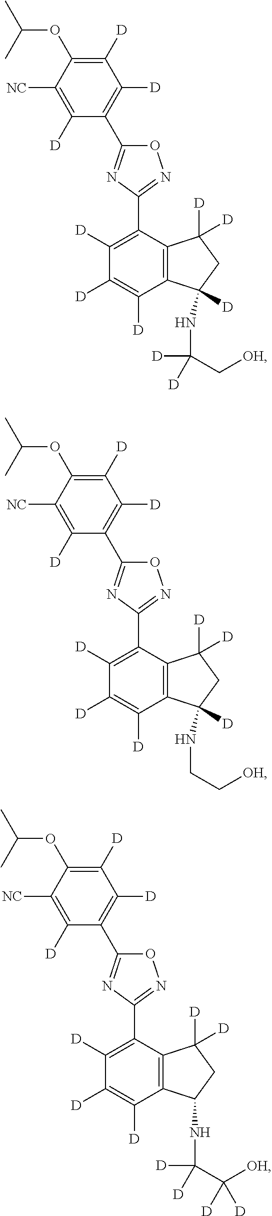 [SMILES: [2H]c1c([2H])c(-c2noc(-c3c([2H])c([2H])c(OC(C)C)c(C#N)c3[2H])n2)c2c(c1[2H])[C@@]([2H])(NC([2H])([2H])CO)CC2([2H])[2H] ; [2H]c1c([2H])c(-c2noc(-c3c([2H])c([2H])c(OC(C)C)c(C#N)c3[2H])n2)c2c(c1[2H])[C@@]([2H])(NCCO)CC2([2H])[2H] ; [2H]c1c([2H])c(-c2noc(-c3c([2H])c([2H])c(OC(C)C)c(C#N)c3[2H])n2)c2c(c1[2H])[C@@H](NC([2H])([2H])C([2H])([2H])O)CC2([2H])[2H]]

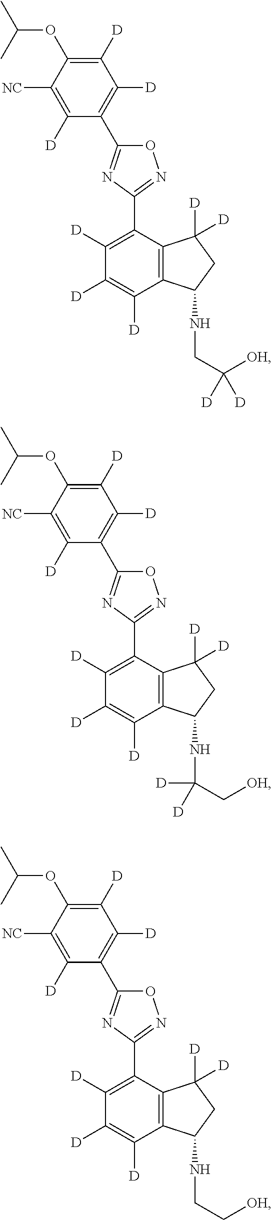 [SMILES: [2H]c1c([2H])c(-c2noc(-c3c([2H])c([2H])c(OC(C)C)c(C#N)c3[2H])n2)c2c(c1[2H])[C@@H](NCC([2H])([2H])O)CC2([2H])[2H] ; [2H]c1c([2H])c(-c2noc(-c3c([2H])c([2H])c(OC(C)C)c(C#N)c3[2H])n2)c2c(c1[2H])[C@@H](NC([2H])([2H])CO)CC2([2H])[2H] ; [2H]c1c([2H])c(-c2noc(-c3c([2H])c([2H])c(OC(C)C)c(C#N)c3[2H])n2)c2c(c1[2H])[C@@H](NCCO)CC2([2H])[2H]]

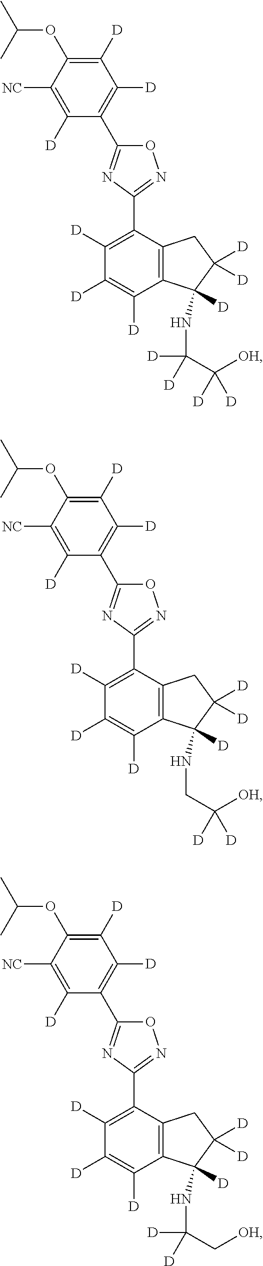 [SMILES: [2H]c1c([2H])c(-c2noc(-c3c([2H])c([2H])c(OC(C)C)c(C#N)c3[2H])n2)c2c(c1[2H])[C@@]([2H])(NC([2H])([2H])C([2H])([2H])O)C([2H])([2H])C2 ; [2H]c1c([2H])c(-c2noc(-c3c([2H])c([2H])c(OC(C)C)c(C#N)c3[2H])n2)c2c(c1[2H])[C@@]([2H])(NCC([2H])([2H])O)C([2H])([2H])C2 ; [2H]c1c([2H])c(-c2noc(-c3c([2H])c([2H])c(OC(C)C)c(C#N)c3[2H])n2)c2c(c1[2H])[C@@]([2H])(NC([2H])([2H])CO)C([2H])([2H])C2]

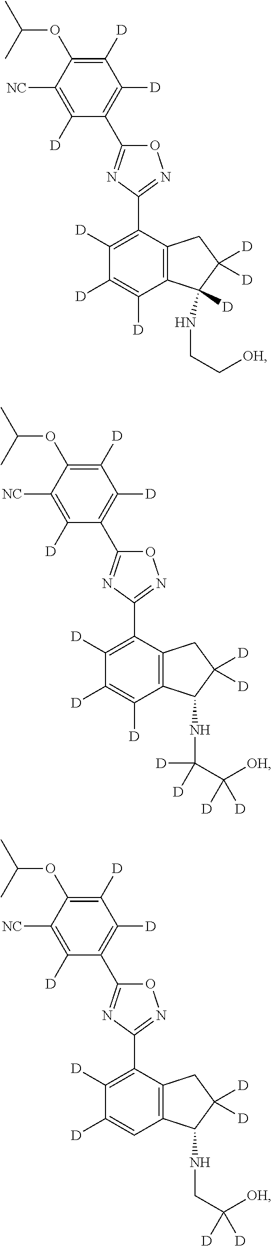 [SMILES: [2H]c1c([2H])c(-c2noc(-c3c([2H])c([2H])c(OC(C)C)c(C#N)c3[2H])n2)c2c(c1[2H])[C@@]([2H])(NCCO)C([2H])([2H])C2 ; [2H]c1c([2H])c(-c2noc(-c3c([2H])c([2H])c(OC(C)C)c(C#N)c3[2H])n2)c2c(c1[2H])[C@@H](NC([2H])([2H])C([2H])([2H])O)C([2H])([2H])C2 ; [2H]c1cc2c(c(-c3noc(-c4c([2H])c([2H])c(OC(C)C)c(C#N)c4[2H])n3)c1[2H])CC([2H])([2H])[C@@H]2NCC([2H])([2H])O]

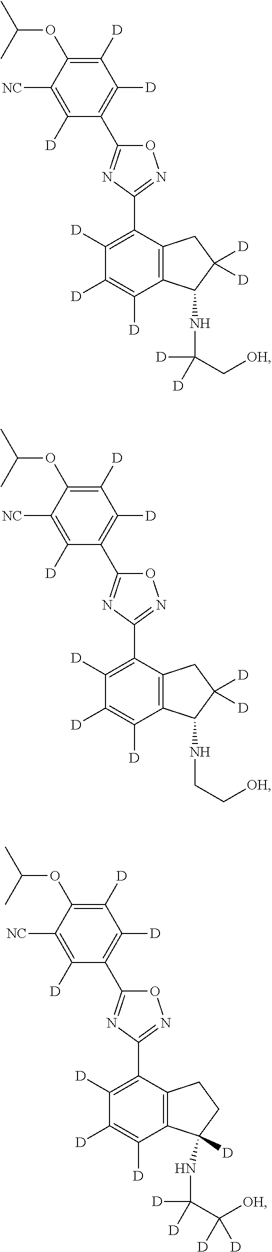 [SMILES: [2H]c1c([2H])c(-c2noc(-c3c([2H])c([2H])c(OC(C)C)c(C#N)c3[2H])n2)c2c(c1[2H])[C@@H](NC([2H])([2H])CO)C([2H])([2H])C2 ; [2H]c1c([2H])c(-c2noc(-c3c([2H])c([2H])c(OC(C)C)c(C#N)c3[2H])n2)c2c(c1[2H])[C@@H](NCCO)C([2H])([2H])C2 ; [2H]c1c([2H])c(-c2noc(-c3c([2H])c([2H])c(OC(C)C)c(C#N)c3[2H])n2)c2c(c1[2H])[C@@]([2H])(NC([2H])([2H])C([2H])([2H])O)CC2]

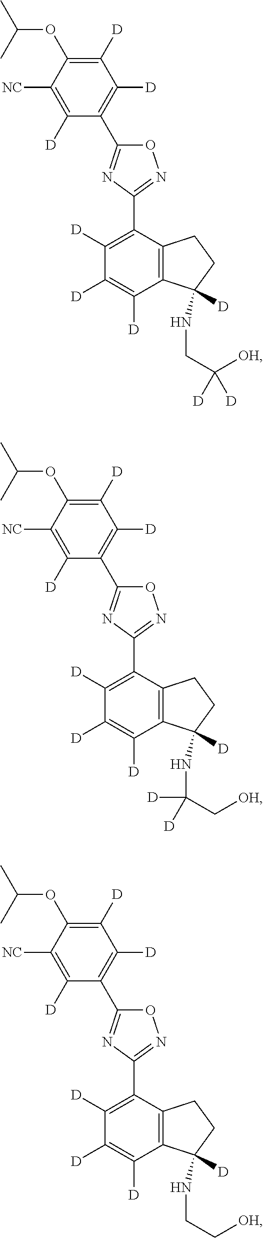 [SMILES: [2H]c1c([2H])c(-c2noc(-c3c([2H])c([2H])c(OC(C)C)c(C#N)c3[2H])n2)c2c(c1[2H])[C@@]([2H])(NCC([2H])([2H])O)CC2 ; [2H]c1c([2H])c(-c2noc(-c3c([2H])c([2H])c(OC(C)C)c(C#N)c3[2H])n2)c2c(c1[2H])[C@@]([2H])(NC([2H])([2H])CO)CC2 ; [2H]c1c([2H])c(-c2noc(-c3c([2H])c([2H])c(OC(C)C)c(C#N)c3[2H])n2)c2c(c1[2H])[C@@]([2H])(NCCO)CC2]

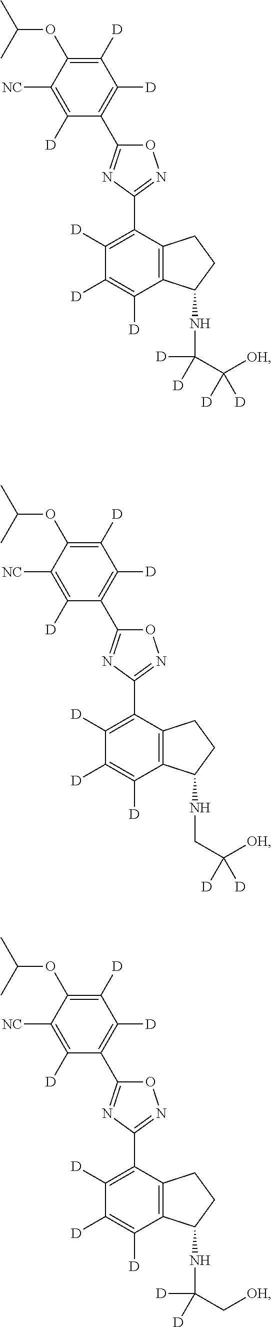 [SMILES: [2H]c1c([2H])c(-c2noc(-c3c([2H])c([2H])c(OC(C)C)c(C#N)c3[2H])n2)c2c(c1[2H])[C@@H](NC([2H])([2H])C([2H])([2H])O)CC2 ; [2H]c1c([2H])c(-c2noc(-c3c([2H])c([2H])c(OC(C)C)c(C#N)c3[2H])n2)c2c(c1[2H])[C@@H](NCC([2H])([2H])O)CC2 ; [2H]c1c([2H])c(-c2noc(-c3c([2H])c([2H])c(OC(C)C)c(C#N)c3[2H])n2)c2c(c1[2H])[C@@H](NC([2H])([2H])CO)CC2]

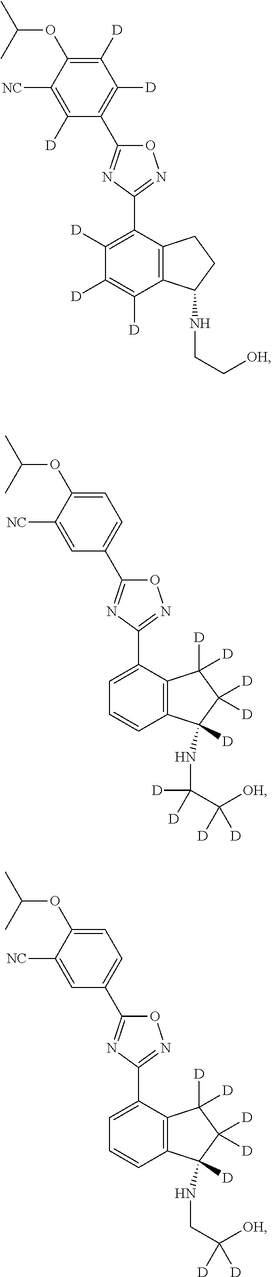 [SMILES: [2H]c1c([2H])c(-c2noc(-c3c([2H])c([2H])c(OC(C)C)c(C#N)c3[2H])n2)c2c(c1[2H])[C@@H](NCCO)CC2 ; [2H]C([2H])(O)C([2H])([2H])N[C@]1([2H])c2cccc(-c3noc(-c4ccc(OC(C)C)c(C#N)c4)n3)c2C([2H])([2H])C1([2H])[2H] ; [2H]C([2H])(O)CN[C@]1([2H])c2cccc(-c3noc(-c4ccc(OC(C)C)c(C#N)c4)n3)c2C([2H])([2H])C1([2H])[2H]]

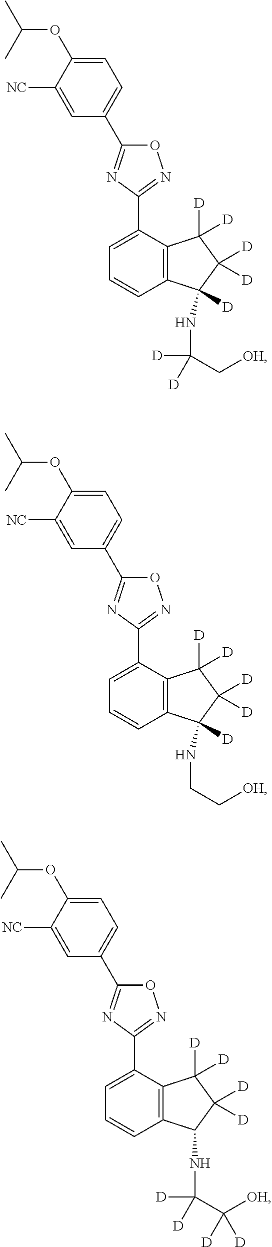 [SMILES: [2H]C([2H])(CO)N[C@]1([2H])c2cccc(-c3noc(-c4ccc(OC(C)C)c(C#N)c4)n3)c2C([2H])([2H])C1([2H])[2H] ; [2H]C1([2H])c2c(-c3noc(-c4ccc(OC(C)C)c(C#N)c4)n3)cccc2[C@@]([2H])(NCCO)C1([2H])[2H] ; [2H]C([2H])(O)C([2H])([2H])N[C@@H]1c2cccc(-c3noc(-c4ccc(OC(C)C)c(C#N)c4)n3)c2C([2H])([2H])C1([2H])[2H]]

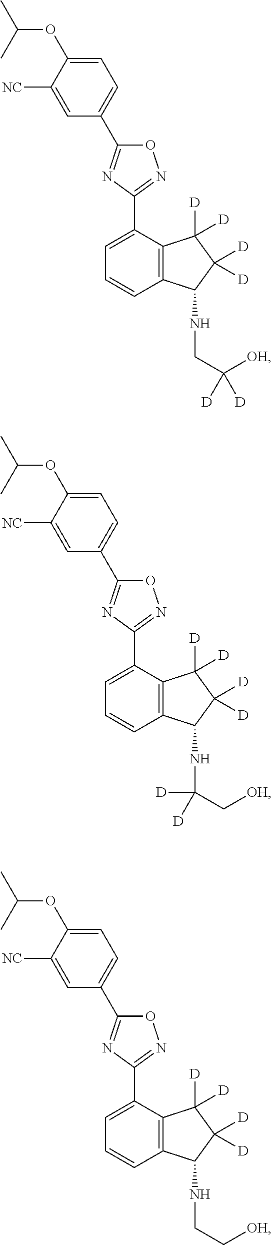 [SMILES: [2H]C([2H])(O)CN[C@@H]1c2cccc(-c3noc(-c4ccc(OC(C)C)c(C#N)c4)n3)c2C([2H])([2H])C1([2H])[2H] ; [2H]C([2H])(CO)N[C@@H]1c2cccc(-c3noc(-c4ccc(OC(C)C)c(C#N)c4)n3)c2C([2H])([2H])C1([2H])[2H] ; [2H]C1([2H])c2c(-c3noc(-c4ccc(OC(C)C)c(C#N)c4)n3)cccc2[C@@H](NCCO)C1([2H])[2H]]

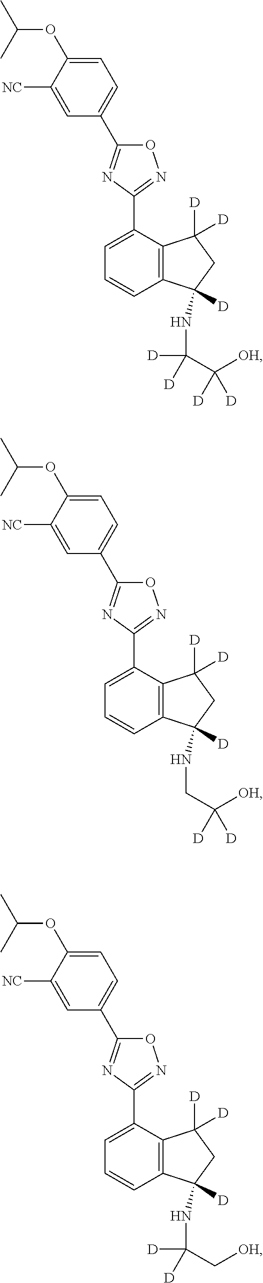 [SMILES: [2H]C1([2H])C[C@]([2H])(NC([2H])([2H])C([2H])([2H])O)c2cccc(-c3noc(-c4ccc(OC(C)C)c(C#N)c4)n3)c21 ; [2H]C([2H])(O)CN[C@@]1([2H])CC([2H])([2H])c2c(-c3noc(-c4ccc(OC(C)C)c(C#N)c4)n3)cccc21 ; [2H]C([2H])(CO)N[C@@]1([2H])CC([2H])([2H])c2c(-c3noc(-c4ccc(OC(C)C)c(C#N)c4)n3)cccc21]

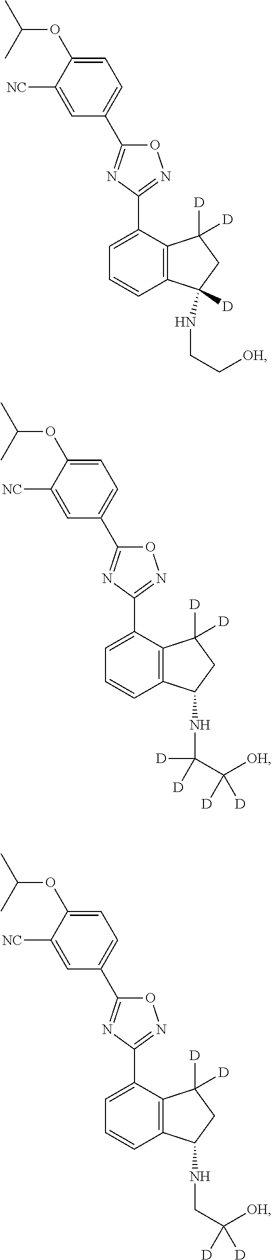 [SMILES: [2H]C1([2H])C[C@]([2H])(NCCO)c2cccc(-c3noc(-c4ccc(OC(C)C)c(C#N)c4)n3)c21 ; [2H]C1([2H])C[C@H](NC([2H])([2H])C([2H])([2H])O)c2cccc(-c3noc(-c4ccc(OC(C)C)c(C#N)c4)n3)c21 ; [2H]C([2H])(O)CN[C@H]1CC([2H])([2H])c2c(-c3noc(-c4ccc(OC(C)C)c(C#N)c4)n3)cccc21]

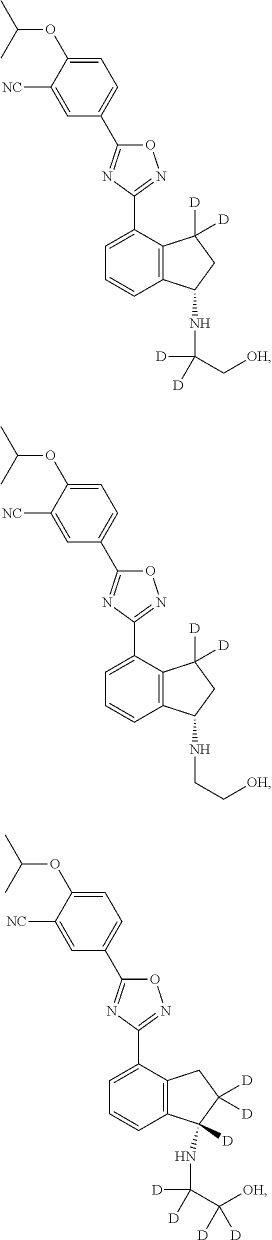 [SMILES: [2H]C([2H])(CO)N[C@H]1CC([2H])([2H])c2c(-c3noc(-c4ccc(OC(C)C)c(C#N)c4)n3)cccc21 ; [2H]C1([2H])C[C@H](NCCO)c2cccc(-c3noc(-c4ccc(OC(C)C)c(C#N)c4)n3)c21 ; [2H]C([2H])(O)C([2H])([2H])N[C@]1([2H])c2cccc(-c3noc(-c4ccc(OC(C)C)c(C#N)c4)n3)c2CC1([2H])[2H]]

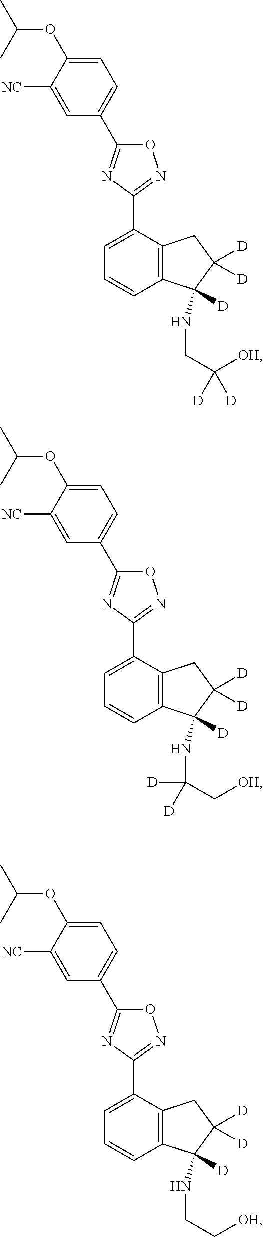 [SMILES: [2H]C([2H])(O)CN[C@]1([2H])c2cccc(-c3noc(-c4ccc(OC(C)C)c(C#N)c4)n3)c2CC1([2H])[2H] ; [2H]C([2H])(CO)N[C@]1([2H])c2cccc(-c3noc(-c4ccc(OC(C)C)c(C#N)c4)n3)c2CC1([2H])[2H] ; [2H]C1([2H])Cc2c(-c3noc(-c4ccc(OC(C)C)c(C#N)c4)n3)cccc2[C@@]1([2H])NCCO]

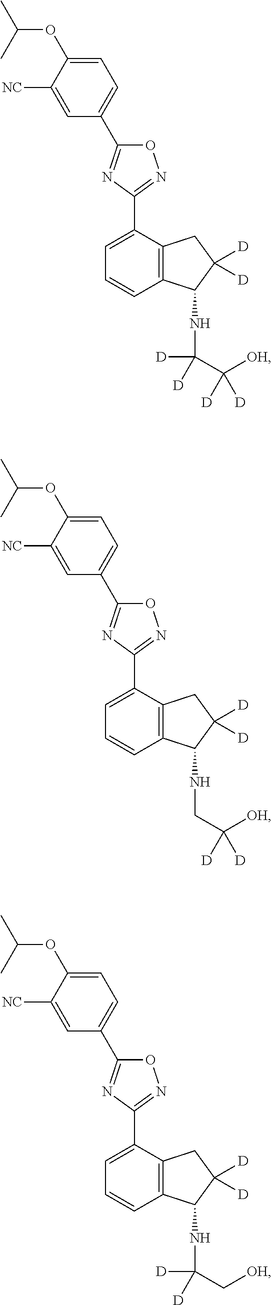 [SMILES: [2H]C1([2H])Cc2c(-c3noc(-c4ccc(OC(C)C)c(C#N)c4)n3)cccc2[C@H]1NC([2H])([2H])C([2H])([2H])O ; [2H]C([2H])(O)CN[C@@H]1c2cccc(-c3noc(-c4ccc(OC(C)C)c(C#N)c4)n3)c2CC1([2H])[2H] ; [2H]C([2H])(CO)N[C@@H]1c2cccc(-c3noc(-c4ccc(OC(C)C)c(C#N)c4)n3)c2CC1([2H])[2H]]

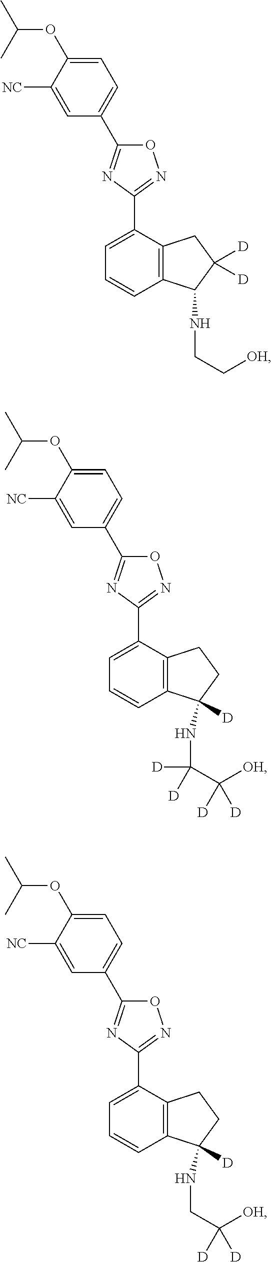 [SMILES: [2H]C1([2H])Cc2c(-c3noc(-c4ccc(OC(C)C)c(C#N)c4)n3)cccc2[C@H]1NCCO ; [2H]C([2H])(O)C([2H])([2H])N[C@@]1([2H])CCc2c(-c3noc(-c4ccc(OC(C)C)c(C#N)c4)n3)cccc21 ; [2H]C([2H])(O)CN[C@@]1([2H])CCc2c(-c3noc(-c4ccc(OC(C)C)c(C#N)c4)n3)cccc21]

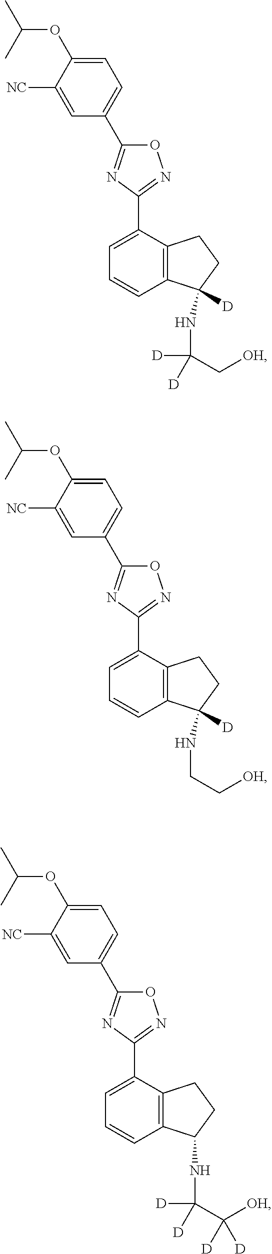 [SMILES: [2H]C([2H])(CO)N[C@@]1([2H])CCc2c(-c3noc(-c4ccc(OC(C)C)c(C#N)c4)n3)cccc21 ; [2H][C@]1(NCCO)CCc2c(-c3noc(-c4ccc(OC(C)C)c(C#N)c4)n3)cccc21 ; [2H]C([2H])(O)C([2H])([2H])N[C@H]1CCc2c(-c3noc(-c4ccc(OC(C)C)c(C#N)c4)n3)cccc21]

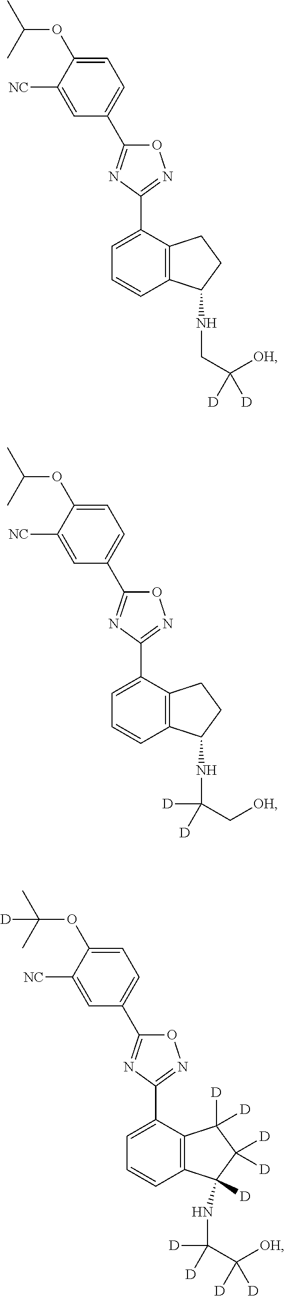 [SMILES: [2H]C([2H])(O)CN[C@H]1CCc2c(-c3noc(-c4ccc(OC(C)C)c(C#N)c4)n3)cccc21 ; [2H]C([2H])(CO)N[C@H]1CCc2c(-c3noc(-c4ccc(OC(C)C)c(C#N)c4)n3)cccc21 ; [2H]C(C)(C)Oc1ccc(-c2nc(-c3cccc4c3C([2H])([2H])C([2H])([2H])[C@]4([2H])NC([2H])([2H])C([2H])([2H])O)no2)cc1C#N]

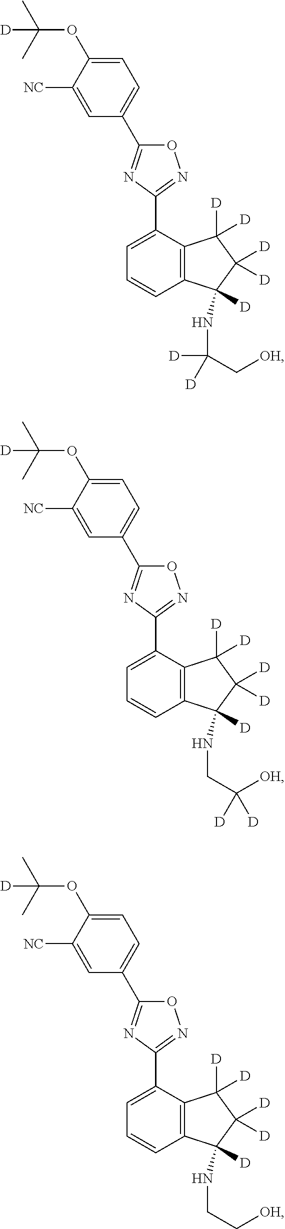 [SMILES: [2H]C([2H])(CO)N[C@]1([2H])c2cccc(-c3noc(-c4ccc(OC([2H])(C)C)c(C#N)c4)n3)c2C([2H])([2H])C1([2H])[2H] ; [2H]C([2H])(O)CN[C@]1([2H])c2cccc(-c3noc(-c4ccc(OC([2H])(C)C)c(C#N)c4)n3)c2C([2H])([2H])C1([2H])[2H] ; [2H]C(C)(C)Oc1ccc(-c2nc(-c3cccc4c3C([2H])([2H])C([2H])([2H])[C@]4([2H])NCCO)no2)cc1C#N]

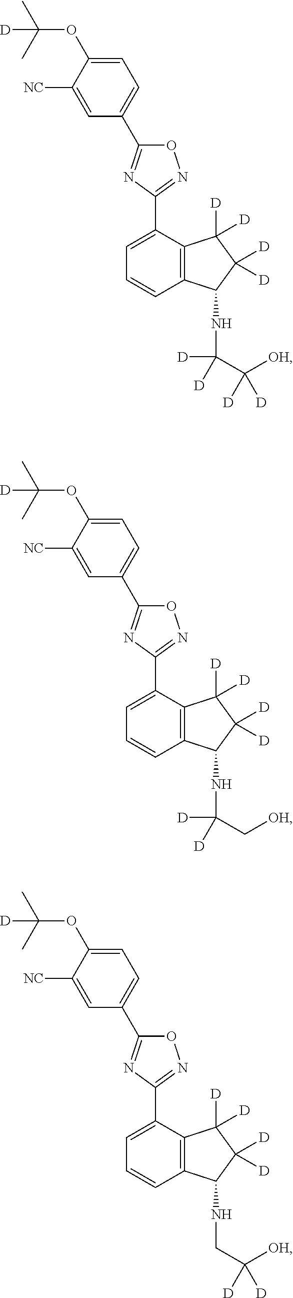 [SMILES: [2H]C(C)(C)Oc1ccc(-c2nc(-c3cccc4c3C([2H])([2H])C([2H])([2H])[C@@H]4NC([2H])([2H])C([2H])([2H])O)no2)cc1C#N ; [2H]C([2H])(CO)N[C@@H]1c2cccc(-c3noc(-c4ccc(OC([2H])(C)C)c(C#N)c4)n3)c2C([2H])([2H])C1([2H])[2H] ; [2H]C([2H])(O)CN[C@@H]1c2cccc(-c3noc(-c4ccc(OC([2H])(C)C)c(C#N)c4)n3)c2C([2H])([2H])C1([2H])[2H]]

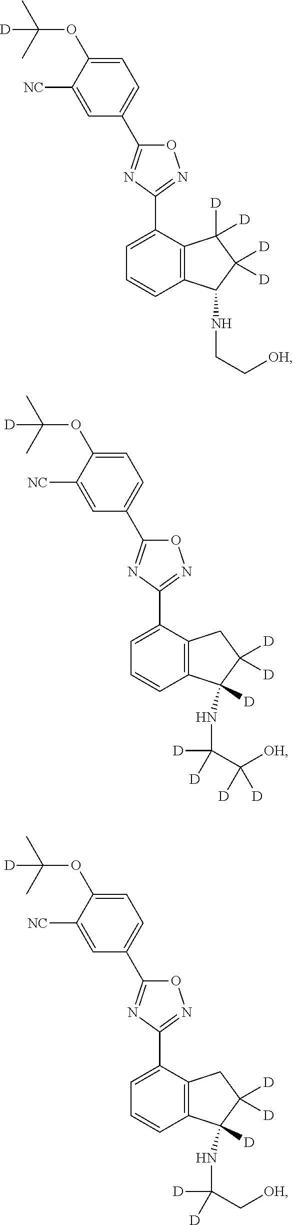 [SMILES: [2H]C(C)(C)Oc1ccc(-c2nc(-c3cccc4c3C([2H])([2H])C([2H])([2H])[C@@H]4NCCO)no2)cc1C#N ; [2H]C(C)(C)Oc1ccc(-c2nc(-c3cccc4c3CC([2H])([2H])[C@]4([2H])NC([2H])([2H])C([2H])([2H])O)no2)cc1C#N ; [2H]C([2H])(CO)N[C@]1([2H])c2cccc(-c3noc(-c4ccc(OC([2H])(C)C)c(C#N)c4)n3)c2CC1([2H])[2H]]

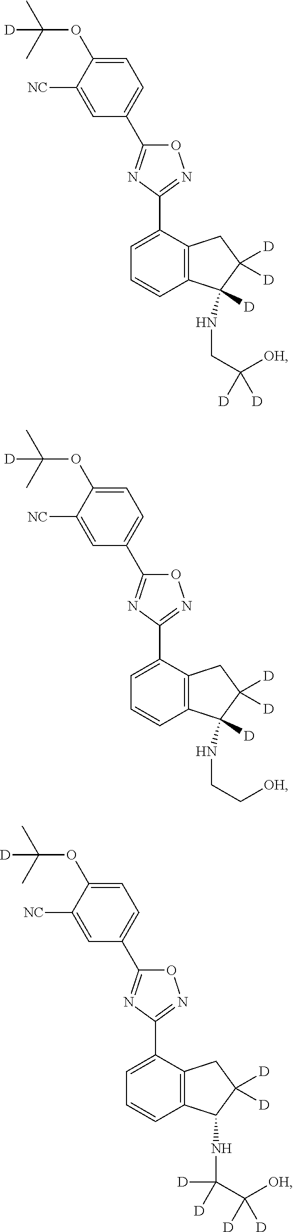 [SMILES: [2H]C([2H])(O)CN[C@]1([2H])c2cccc(-c3noc(-c4ccc(OC([2H])(C)C)c(C#N)c4)n3)c2CC1([2H])[2H] ; [2H]C(C)(C)Oc1ccc(-c2nc(-c3cccc4c3CC([2H])([2H])[C@]4([2H])NCCO)no2)cc1C#N ; [2H]C(C)(C)Oc1ccc(-c2nc(-c3cccc4c3CC([2H])([2H])[C@@H]4NC([2H])([2H])C([2H])([2H])O)no2)cc1C#N]

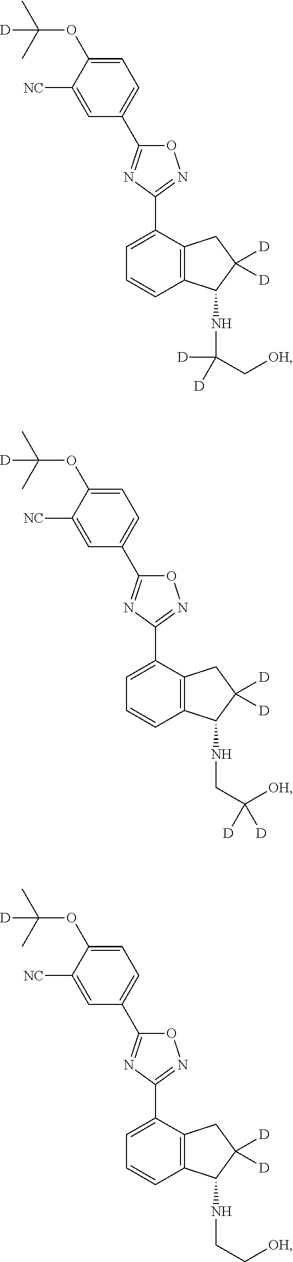 [SMILES: [2H]C([2H])(CO)N[C@@H]1c2cccc(-c3noc(-c4ccc(OC([2H])(C)C)c(C#N)c4)n3)c2CC1([2H])[2H] ; [2H]C([2H])(O)CN[C@@H]1c2cccc(-c3noc(-c4ccc(OC([2H])(C)C)c(C#N)c4)n3)c2CC1([2H])[2H] ; [2H]C(C)(C)Oc1ccc(-c2nc(-c3cccc4c3CC([2H])([2H])[C@@H]4NCCO)no2)cc1C#N]

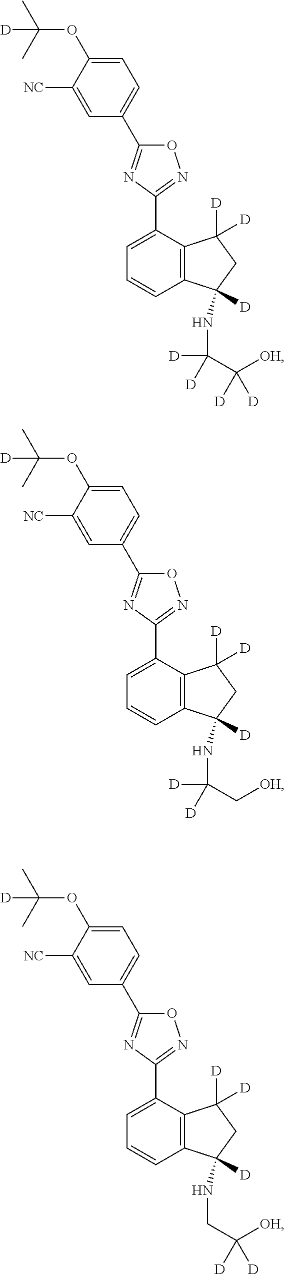 [SMILES: [2H]C(C)(C)Oc1ccc(-c2nc(-c3cccc4c3C([2H])([2H])C[C@]4([2H])NC([2H])([2H])C([2H])([2H])O)no2)cc1C#N ; [2H]C([2H])(CO)N[C@@]1([2H])CC([2H])([2H])c2c(-c3noc(-c4ccc(OC([2H])(C)C)c(C#N)c4)n3)cccc21 ; [2H]C([2H])(O)CN[C@@]1([2H])CC([2H])([2H])c2c(-c3noc(-c4ccc(OC([2H])(C)C)c(C#N)c4)n3)cccc21]

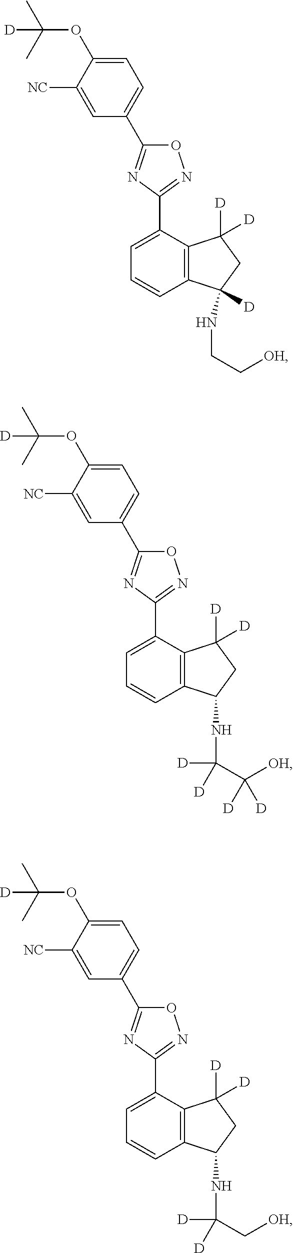 [SMILES: [2H]C(C)(C)Oc1ccc(-c2nc(-c3cccc4c3C([2H])([2H])C[C@]4([2H])NCCO)no2)cc1C#N ; [2H]C(C)(C)Oc1ccc(-c2nc(-c3cccc4c3C([2H])([2H])C[C@@H]4NC([2H])([2H])C([2H])([2H])O)no2)cc1C#N ; [2H]C([2H])(CO)N[C@H]1CC([2H])([2H])c2c(-c3noc(-c4ccc(OC([2H])(C)C)c(C#N)c4)n3)cccc21]

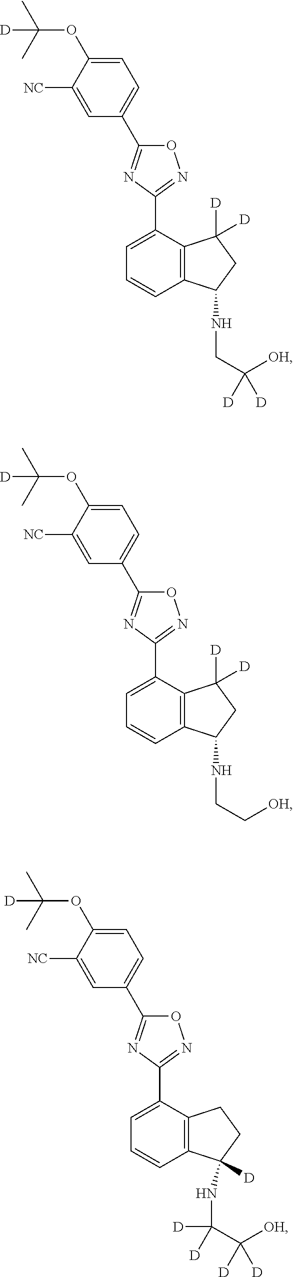 [SMILES: [2H]C([2H])(O)CN[C@H]1CC([2H])([2H])c2c(-c3noc(-c4ccc(OC([2H])(C)C)c(C#N)c4)n3)cccc21 ; [2H]C(C)(C)Oc1ccc(-c2nc(-c3cccc4c3C([2H])([2H])C[C@@H]4NCCO)no2)cc1C#N ; [2H]C(C)(C)Oc1ccc(-c2nc(-c3cccc4c3CC[C@]4([2H])NC([2H])([2H])C([2H])([2H])O)no2)cc1C#N]

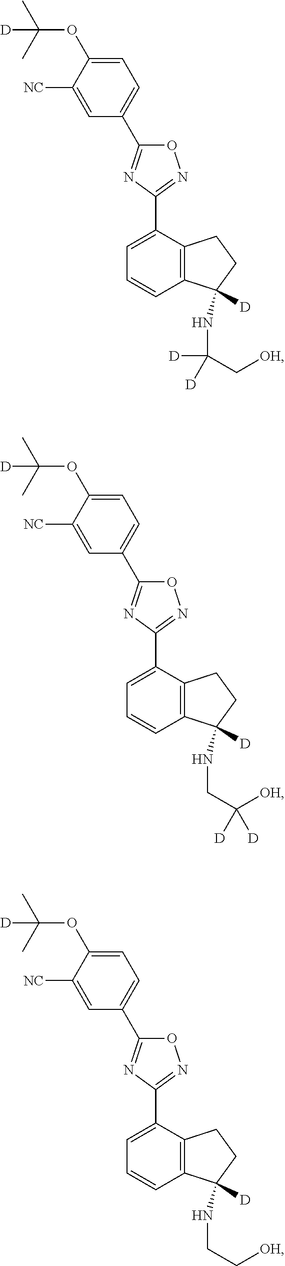 [SMILES: [2H]C([2H])(CO)N[C@@]1([2H])CCc2c(-c3noc(-c4ccc(OC([2H])(C)C)c(C#N)c4)n3)cccc21 ; [2H]C([2H])(O)CN[C@@]1([2H])CCc2c(-c3noc(-c4ccc(OC([2H])(C)C)c(C#N)c4)n3)cccc21 ; [2H]C(C)(C)Oc1ccc(-c2nc(-c3cccc4c3CC[C@]4([2H])NCCO)no2)cc1C#N]

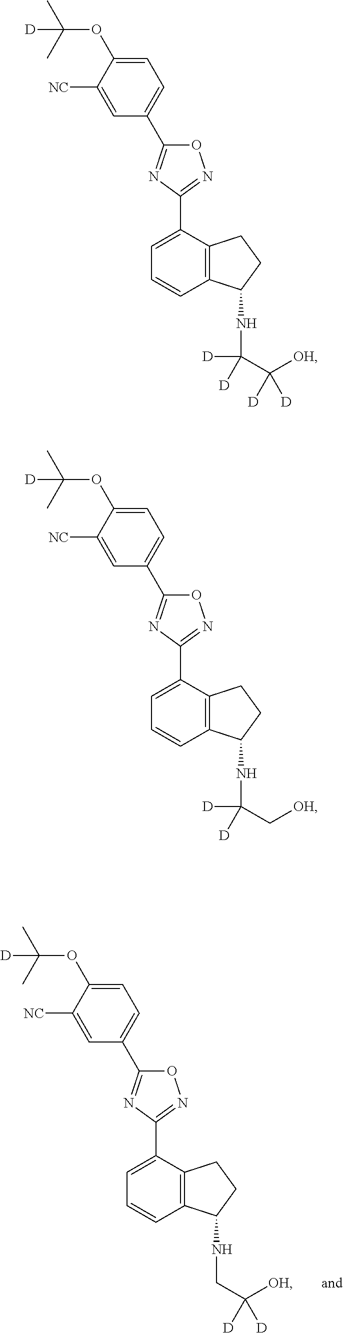 [SMILES: [2H]C(C)(C)Oc1ccc(-c2nc(-c3cccc4c3CC[C@@H]4NC([2H])([2H])C([2H])([2H])O)no2)cc1C#N ; [2H]C([2H])(CO)N[C@H]1CCc2c(-c3noc(-c4ccc(OC([2H])(C)C)c(C#N)c4)n3)cccc21 ; [2H]C([2H])(O)CN[C@H]1CCc2c(-c3noc(-c4ccc(OC([2H])(C)C)c(C#N)c4)n3)cccc21 ; "and"]

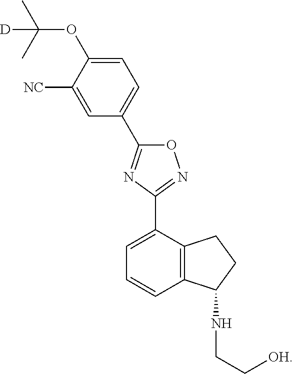 [SMILES: [2H]C(C)(C)Oc1ccc(-c2nc(-c3cccc4c3CC[C@@H]4NCCO)no2)cc1C#N]

Changes in the metabolic properties of the compounds disclosed herein as compared to their non-isotopically enriched analogs can be shown using the following assays. Compounds listed above which have not yet been made and/or tested are predicted to have changed metabolic properties as shown by one or more of these assays as well.

### Biological Activity Assays

### In Vitro Liver Microsomal Stability Assay

Human liver microsomal stability assays were conducted at 2 mg per mL liver microsome protein with an NADPH-generating system consisting of NADP (1 mM, pH 7.4), glucose-5-phosphate (5 mM, pH 7.4), and glucose-6-phosphate dehydrogenase (I unit/mL).

Test compounds were prepared as solutions in DMSO and added to the assay mixture (1 μM, final concentration in incubation) to be incubated at 37±1° C. Reactions were initiated with the addition of cofactor and were stopped at 0, 60, 120, or 240 min after cofactor addition with stop reagent (0.2 mL acetonitrile). Samples were centrifuged (920×g for 10 min at 10° C.) in 96-well plates. Supernatant fractions were analyzed by LC-MS/MS to determine the percent remaining and estimate the degradation half-life of the test compounds. The results are presented below:

| | Clearance % | Half-Life % |
|---|---|---|
| Example # | change over d0 | change over d0 |
| 1 | 0.00 | 0.0 |
| 2 | 1 | −9.0 |
| 3 | 7 | 0.0 |
| 4 | 5 | −4.6 |
| 5 | 11 | 0.0 |
| 6 | 2 | 17.9 |

### In Vitro Metabolism Using Human Cytochrome P450 Enzymes

The cytochrome P450 enzymes are expressed from the corresponding human cDNA using a baculovirus expression system (BD Biosciences, San Jose, Calif.). A 0.25 milliliter reaction mixture containing 0.8 milligrams per milliliter protein, 1.3 millimolar NADP⁺, 3.3 millimolar glucose-6-phosphate, 0.4 U/mL glucose-6-phosphate dehydrogenase, 3.3 millimolar magnesium chloride and 0.2 millimolar of a compound of Formula I, the corresponding non-isotopically enriched compound or standard or control in 100 millimolar potassium phosphate (pH 7.4) is incubated at 37° C. for 20 min. After incubation, the reaction is stopped by the addition of an appropriate solvent (e.g., acetonitrile, 20% trichloroacetic acid, 94% acetonitrile/6% glacial acetic acid, 70% perchloric acid, 94% acetonitrile/6% glacial acetic acid) and centrifuged (10,000 g) for 3 min. The supernatant is analyzed by HPLC/MS/MS.

| Cytochrome P₄₅₀ | Standard |
|---|---|
| CYP1A2 | Phenacetin |
| CYP2A6 | Coumarin |
| CYP2B6 | [¹³C]-(S)-mephenytoin |
| CYP2C8 | Paclitaxel |
| CYP2C9 | Diclofenac |
| CYP2C19 | [¹³C]-(S)-mephenytoin |
| CYP2D6 | (+/−)-Bufuralol |
| CYP2E1 | Chlorzoxazone |
| CYP3A4 | Testosterone |
| CYP4A | [¹³C]-Lauric acid |

### Monoamine Oxidase A Inhibition and Oxidative Turnover

The procedure is carried out using the methods described by Weyler, Journal of Biological Chemistry 1985, 260, 13199-13207, which is hereby incorporated by reference in its entirety. Monoamine oxidase A activity is measured spectrophotometrically by monitoring the increase in absorbance at 314 nm on oxidation of kynuramine with formation of 4-hydroxyquinoline. The measurements are carried out, at 30° C., in 50 mM NaPᵢ buffer, pH 7.2, containing 0.2% Triton X-100 (monoamine oxidase assay buffer), plus 1 mM kynuramine, and the desired amount of enzyme in 1 mL total volume.

### Monooamine Oxidase B Inhibition and Oxidative Turnover

The procedure is carried out as described in Uebelhack, Pharmacopsychiatry 1998, 31(5), 187-192, which is hereby incorporated by reference in its entirety.

### Experimental Procedures for Studying Agonist-Induced Internalization, Receptor Phosphorylation and Receptor Polyubiquitination in Stably Expressed S1P1-GFP Cells

The procedure is carried out as described in U.S. Pat. No. 8,446,183, which is hereby incorporated by reference in its entirety.

S1P1-Mediated Inhibition of cAMP Reporter Assay

The procedure is carried out as described in WO 2011060392, which is hereby incorporated by reference in its entirety.

### Rat Pharmacokinetic Assays

The procedure is carried out as described in WO 2011060392, which is hereby incorporated by reference in its entirety.

### Rat Lymphopenia Assay

The procedure is carried out as described in WO 2011060392, which is hereby incorporated by reference in its entirety.

### Rat Therapeutic Index Determination

The procedure is carried out as described in WO 2011060392, which is hereby incorporated by reference in its entirety.

### TNBS Crohn's Colitis Model in Rats

The procedure is carried out as described in WO 2011060392, which is hereby incorporated by reference in its entirety.

### Influenza A H1N1 Model in Mice

The procedure is carried out as described in WO 2011060392, which is hereby incorporated by reference in its entirety.

From the foregoing description, one skilled in the art can easily ascertain the essential characteristics of this invention, and without departing from the spirit and scope thereof, can make various changes and modifications of the invention to adapt it to various usages and conditions.

What is claimed is:

1-56. (canceled)

57. A compound of structural Formula I 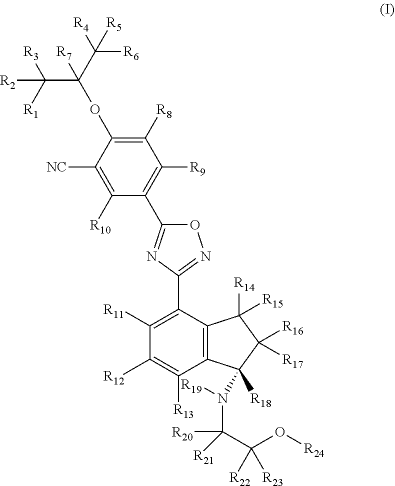
or a salt thereof, wherein:
R₁-R₂₄ are independently selected from the group consisting of hydrogen and deuterium; and
at least one of R₁-R₂₄ is deuterium or contains deuterium.

58. The compound as recited in claim 57, wherein R₇ is deuterium.

59. The compound as recited in claim 57, wherein R₁-R₆ are deuterium.

60. The compound as recited in claim 57, wherein R₁-R₇ are deuterium.

61. The compound as recited in claim 57, wherein R₁₈ is deuterium.

62. The compound as recited in claim 57, wherein R₇ and R₁₈ are deuterium.

63. The compound as recited in claim 57, wherein R₂₀-R₂₁ are deuterium.

64. The compound as recited in claim 57, wherein R₂₂-R₂₃ are deuterium.

65. The compound as recited in claim 57, wherein R₂₀-R₂₃ are deuterium.

66. The compound as recited in claim 57, wherein R₇ and R₂₀-R₂₃ are deuterium.

67. The compound as recited in claim 57, wherein R₁-R₇ and R₂₀-R₂₃ are deuterium.

68. The compound as recited in claim 57 wherein at least one of R₁-R₁₃ independently has deuterium enrichment of no less than about 10%.

69. The compound as recited in claim 57 wherein said compound has a structural formula selected from the group consisting of 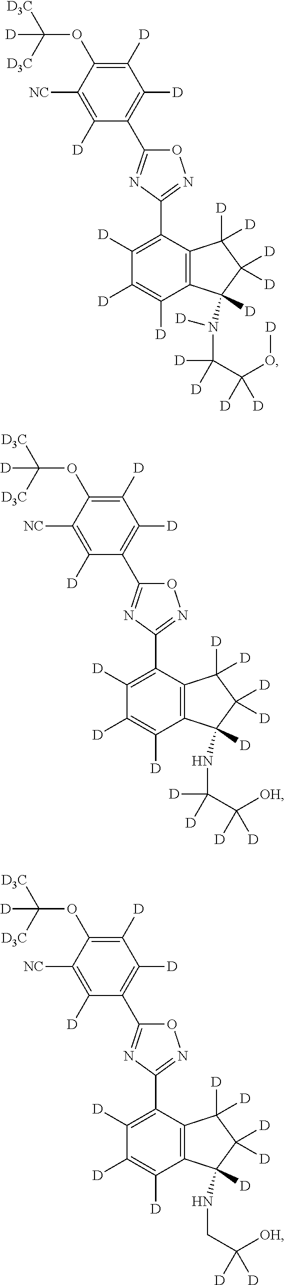 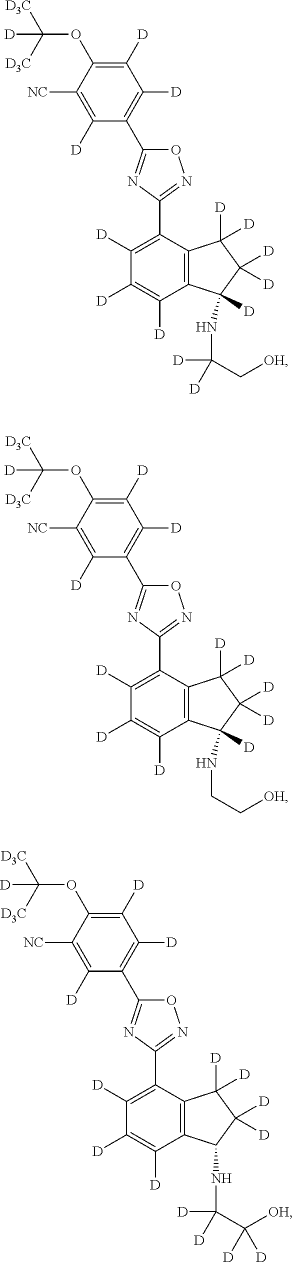 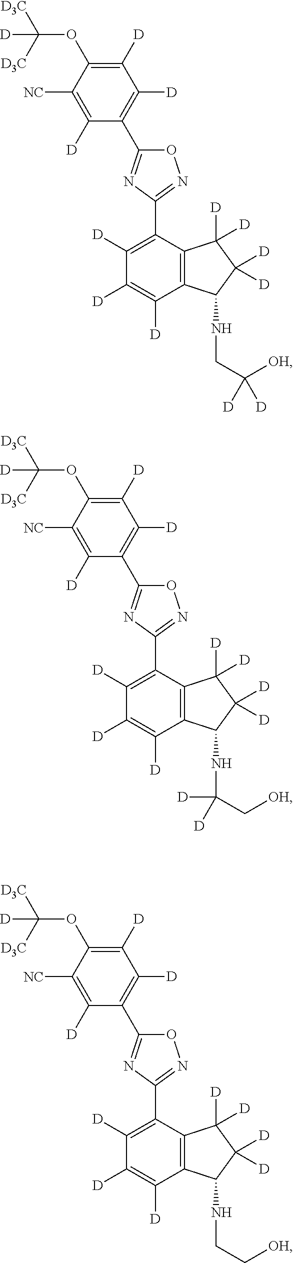 [SMILES: [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@H](NCC([2H])([2H])O)C([2H])([2H])C2([2H])[2H] ; [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@H](NC([2H])([2H])CO)C([2H])([2H])C2([2H])[2H] ; [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@H](NCCO)C([2H])([2H])C2([2H])[2H]] 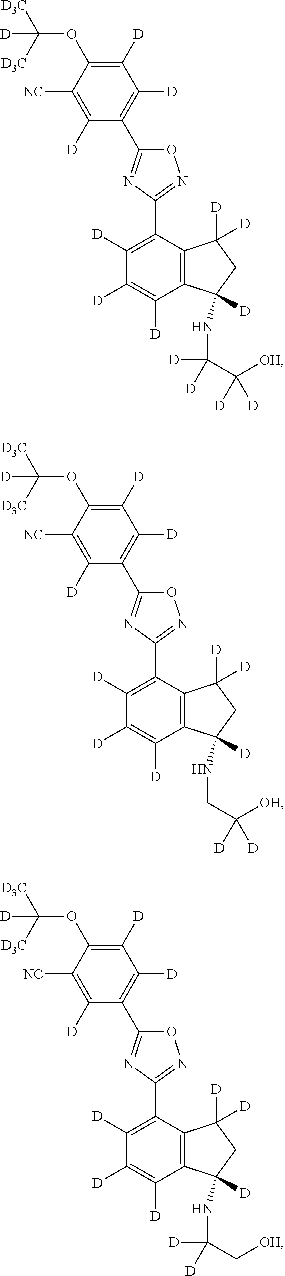 [SMILES: [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@]([2H])(NC([2H])([2H])C([2H])([2H])O)CC2([2H])[2H] ; [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@]([2H])(NCC([2H])([2H])O)CC2([2H])[2H] ; [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@]([2H])(NC([2H])([2H])CO)CC2([2H])[2H]] 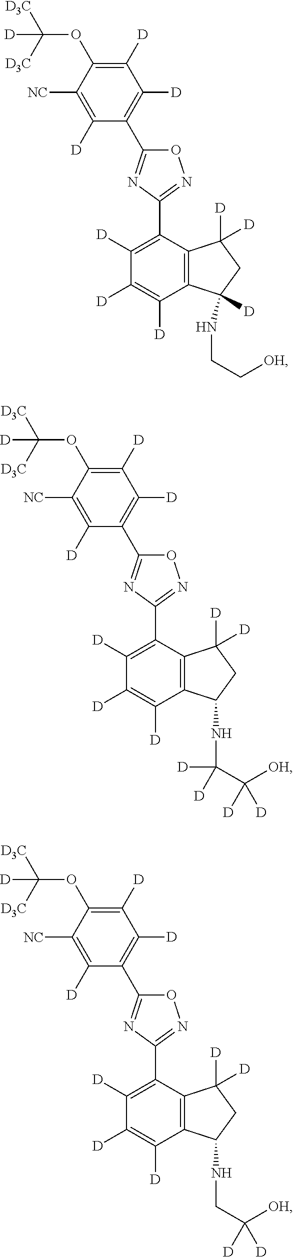 [SMILES: [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@]([2H])(NCCO)CC2([2H])[2H] ; [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@H](NC([2H])([2H])C([2H])([2H])O)CC2([2H])[2H] ; [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@H](NCC([2H])([2H])O)CC2([2H])[2H]] 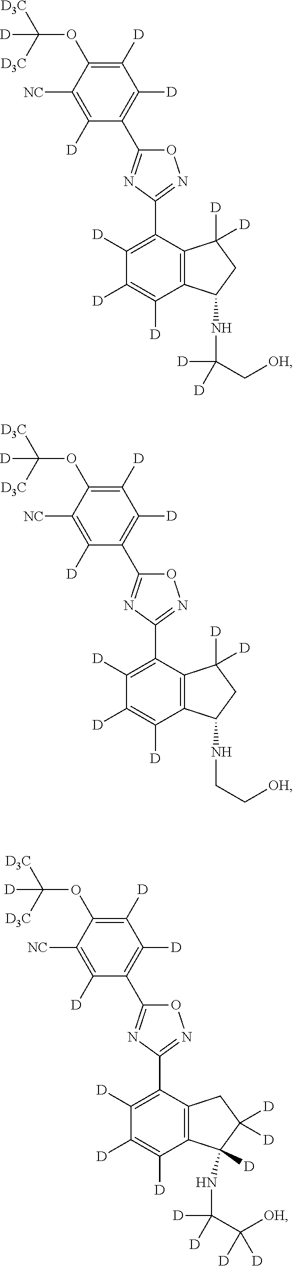 [SMILES: [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@H](NC([2H])([2H])CO)CC2([2H])[2H] ; [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@H](NCCO)CC2([2H])[2H] ; [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@]([2H])(NC([2H])([2H])C([2H])([2H])O)C([2H])([2H])C2] 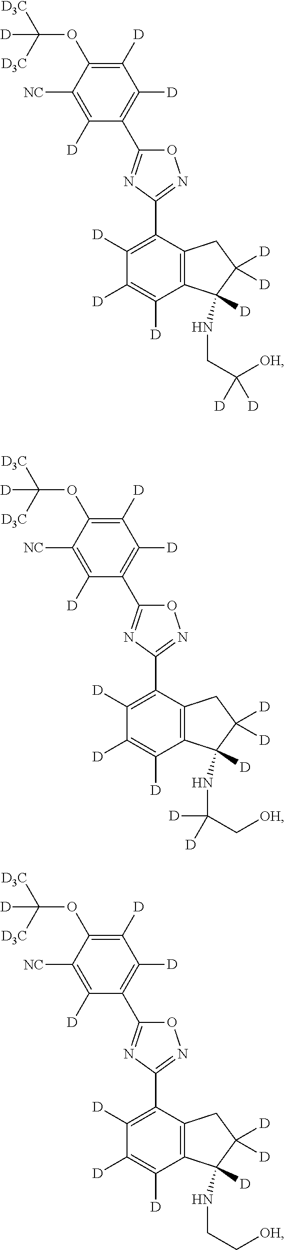 [SMILES: [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@]([2H])(NCC([2H])([2H])O)C([2H])([2H])C2 ; [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@]([2H])(NC([2H])([2H])CO)C([2H])([2H])C2 ; [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@]([2H])(NCCO)C([2H])([2H])C2] 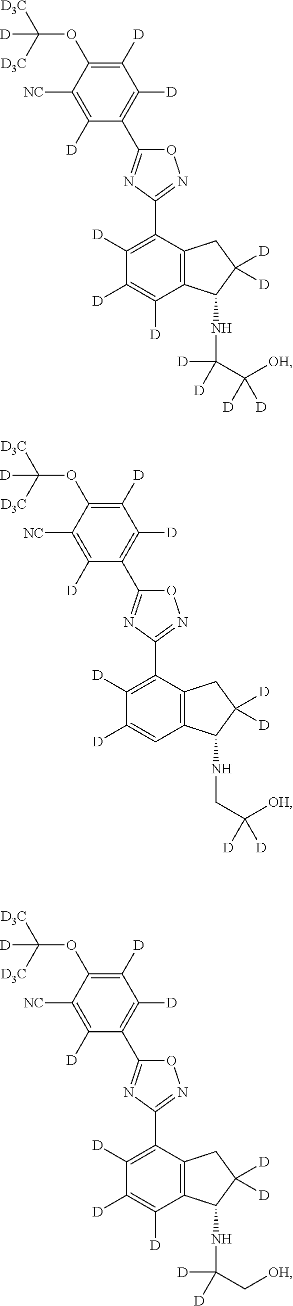 [SMILES: [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@H](NC([2H])([2H])C([2H])([2H])O)C([2H])([2H])C2 ; [2H]c1cc2c(c(-c3noc(-c4c([2H])c([2H])c(OC([2H])(C([2H])([2H])[2H])C([2H])([2H])[2H])c(C#N)c4[2H])n3)c1[2H])CC([2H])([2H])[C@@H]2NCC([2H])([2H])O ; [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@H](NC([2H])([2H])CO)C([2H])([2H])C2] 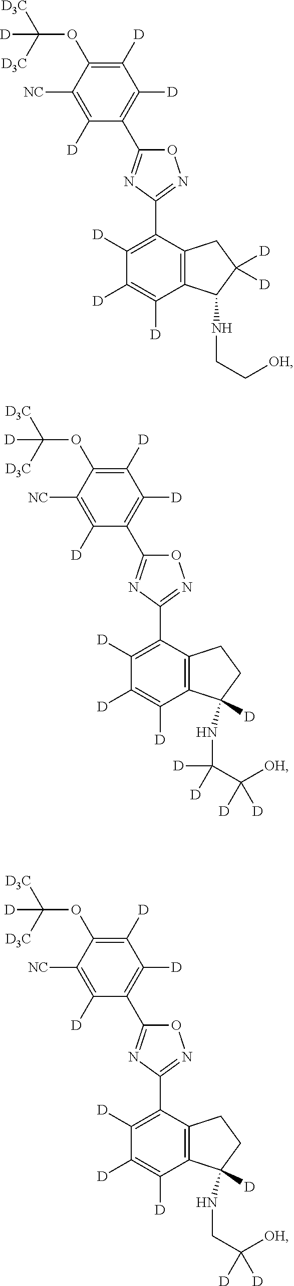 [SMILES: [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@H](NCCO)C([2H])([2H])C2 ; [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@]([2H])(NC([2H])([2H])C([2H])([2H])O)CC2 ; [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@]([2H])(NCC([2H])([2H])O)CC2] 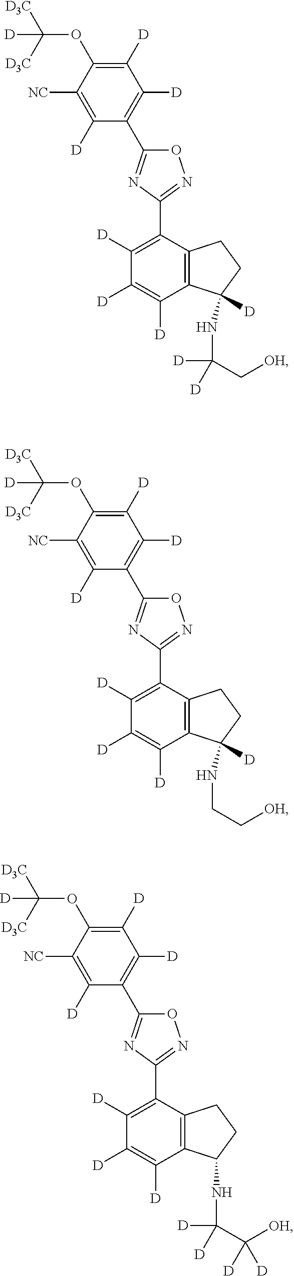 [SMILES: [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@]([2H])(NC([2H])([2H])CO)CC2 ; [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@]([2H])(NCCO)CC2 ; [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@H](NC([2H])([2H])C([2H])([2H])O)CC2] 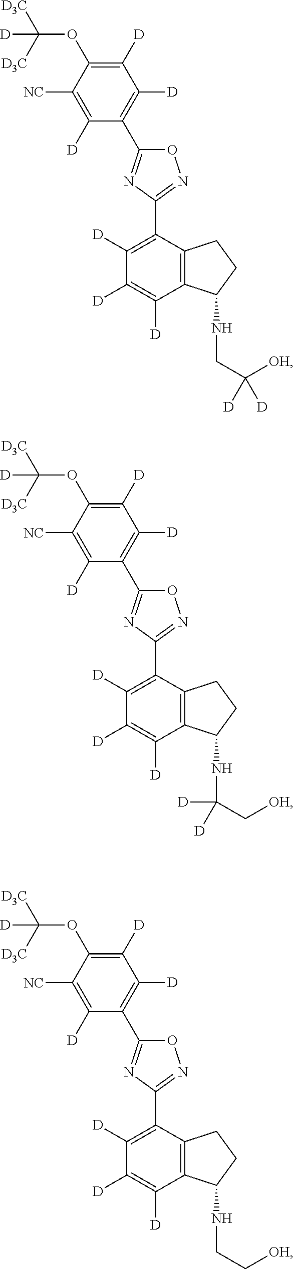 [SMILES: [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@H](NCC([2H])([2H])O)CC2 ; [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@H](NC([2H])([2H])CO)CC2 ; [2H]c1c([2H])c(-c2noc(-c3c([2H])c([2H])c(OC([2H])(C([2H])([2H])[2H])C([2H])([2H])[2H])c(C#N)c3[2H])n2)c2c(c1[2H])[C@@H](NCCO)CC2] 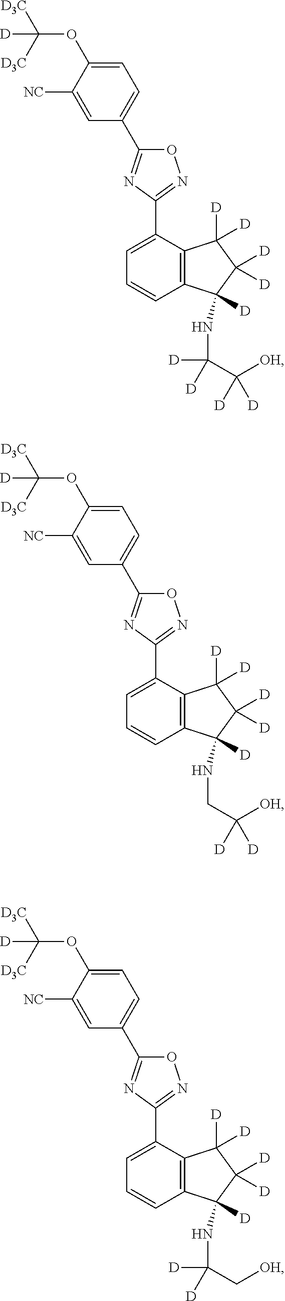 [SMILES: [2H]C([2H])([2H])C([2H])(Oc1ccc(-c2nc(-c3cccc4c3C([2H])([2H])C([2H])([2H])[C@]4([2H])NC([2H])([2H])C([2H])([2H])O)no2)cc1C#N)C([2H])([2H])[2H] ; [2H]C([2H])(O)CN[C@]1([2H])c2cccc(-c3noc(-c4ccc(OC([2H])(C([2H])([2H])[2H])C([2H])([2H])[2H])c(C#N)c4)n3)c2C([2H])([2H])C1([2H])[2H] ; [2H]C([2H])(CO)N[C@]1([2H])c2cccc(-c3noc(-c4ccc(OC([2H])(C([2H])([2H])[2H])C([2H])([2H])[2H])c(C#N)c4)n3)c2C([2H])([2H])C1([2H])[2H]] 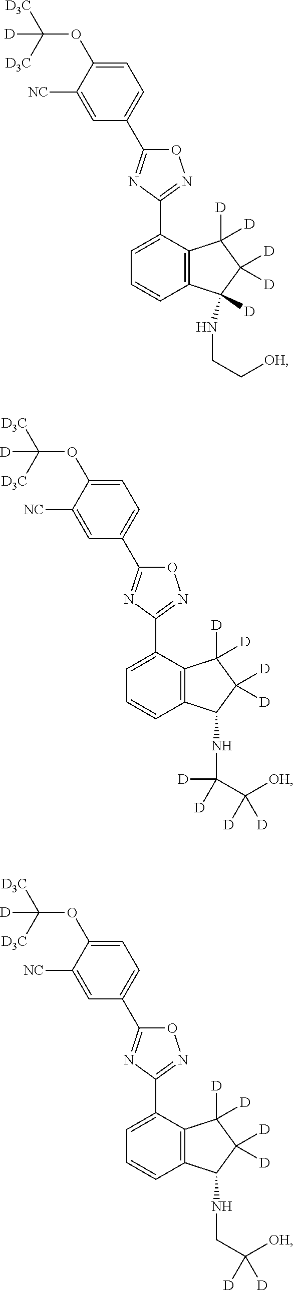 [SMILES: [2H]C([2H])([2H])C([2H])(Oc1ccc(-c2nc(-c3cccc4c3C([2H])([2H])C([2H])([2H])[C@]4([2H])NCCO)no2)cc1C#N)C([2H])([2H])[2H] ; [2H]C([2H])([2H])C([2H])(Oc1ccc(-c2nc(-c3cccc4c3C([2H])([2H])C([2H])([2H])[C@@H]4NC([2H])([2H])C([2H])([2H])O)no2)cc1C#N)C([2H])([2H])[2H] ; [2H]C([2H])(O)CN[C@@H]1c2cccc(-c3noc(-c4ccc(OC([2H])(C([2H])([2H])[2H])C([2H])([2H])[2H])c(C#N)c4)n3)c2C([2H])([2H])C1([2H])[2H]] 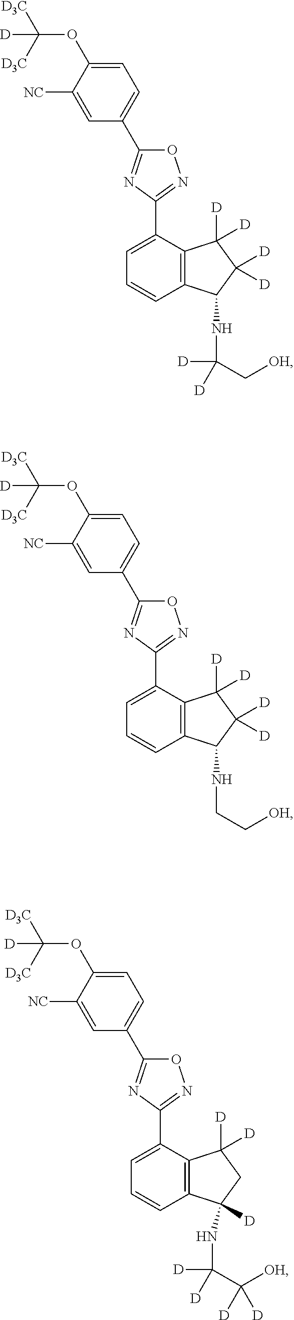 [SMILES: [2H]C([2H])(CO)N[C@@H]1c2cccc(-c3noc(-c4ccc(OC([2H])(C([2H])([2H])[2H])C([2H])([2H])[2H])c(C#N)c4)n3)c2C([2H])([2H])C1([2H])[2H] ; [2H]C([2H])([2H])C([2H])(Oc1ccc(-c2nc(-c3cccc4c3C([2H])([2H])C([2H])([2H])[C@@H]4NCCO)no2)cc1C#N)C([2H])([2H])[2H] ; [2H]C1([2H])C[C@]([2H])(NC([2H])([2H])C([2H])([2H])O)c2cccc(-c3noc(-c4ccc(OC([2H])(C([2H])([2H])[2H])C([2H])([2H])[2H])c(C#N)c4)n3)c21] 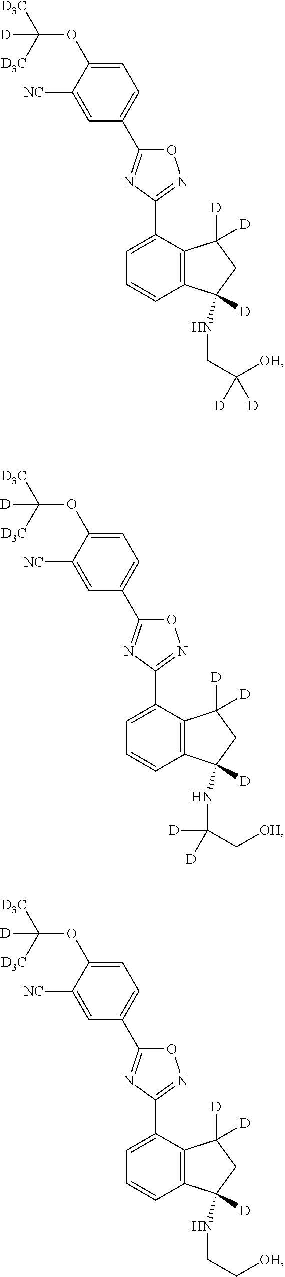 [SMILES: [2H]C([2H])(O)CN[C@@]1([2H])CC([2H])([2H])c2c(-c3noc(-c4ccc(OC([2H])(C([2H])([2H])[2H])C([2H])([2H])[2H])c(C#N)c4)n3)cccc21 ; [2H]C([2H])(CO)N[C@@]1([2H])CC([2H])([2H])c2c(-c3noc(-c4ccc(OC([2H])(C([2H])([2H])[2H])C([2H])([2H])[2H])c(C#N)c4)n3)cccc21 ; [2H]C1([2H])C[C@]([2H])(NCCO)c2cccc(-c3noc(-c4ccc(OC([2H])(C([2H])([2H])[2H])C([2H])([2H])[2H])c(C#N)c4)n3)c21] 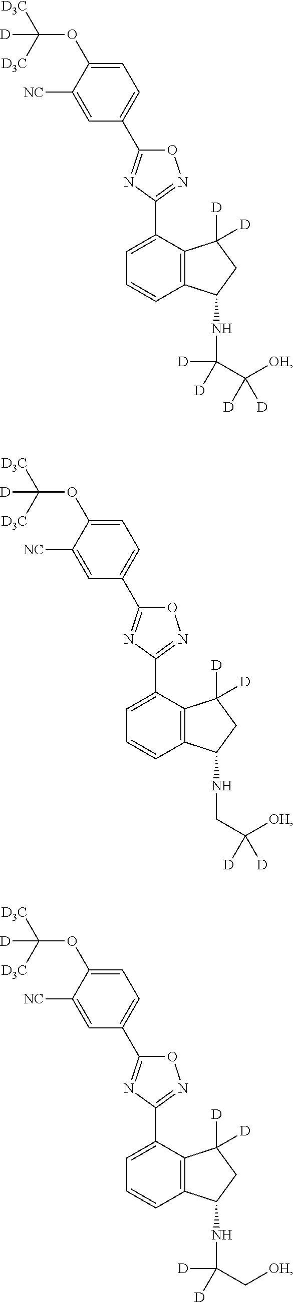 [SMILES: [2H]C1([2H])C[C@H](NC([2H])([2H])C([2H])([2H])O)c2cccc(-c3noc(-c4ccc(OC([2H])(C([2H])([2H])[2H])C([2H])([2H])[2H])c(C#N)c4)n3)c21 ; [2H]C([2H])(O)CN[C@H]1CC([2H])([2H])c2c(-c3noc(-c4ccc(OC([2H])(C([2H])([2H])[2H])C([2H])([2H])[2H])c(C#N)c4)n3)cccc21 ; [2H]C([2H])(CO)N[C@H]1CC([2H])([2H])c2c(-c3noc(-c4ccc(OC([2H])(C([2H])([2H])[2H])C([2H])([2H])[2H])c(C#N)c4)n3)cccc21] 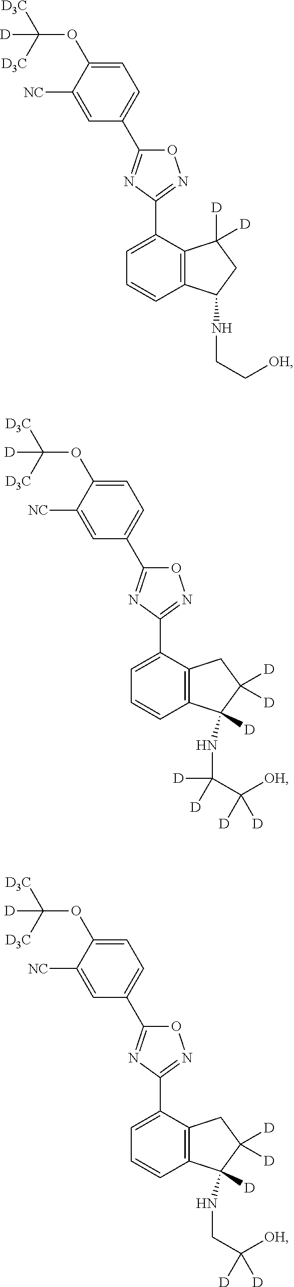 [SMILES: [2H]C1([2H])C[C@H](NCCO)c2cccc(-c3noc(-c4ccc(OC([2H])(C([2H])([2H])[2H])C([2H])([2H])[2H])c(C#N)c4)n3)c21 ; [2H]C([2H])([2H])C([2H])(Oc1ccc(-c2nc(-c3cccc4c3CC([2H])([2H])[C@]4([2H])NC([2H])([2H])C([2H])([2H])O)no2)cc1C#N)C([2H])([2H])[2H] ; [2H]C([2H])(O)CN[C@]1([2H])c2cccc(-c3noc(-c4ccc(OC([2H])(C([2H])([2H])[2H])C([2H])([2H])[2H])c(C#N)c4)n3)c2CC1([2H])[2H]] 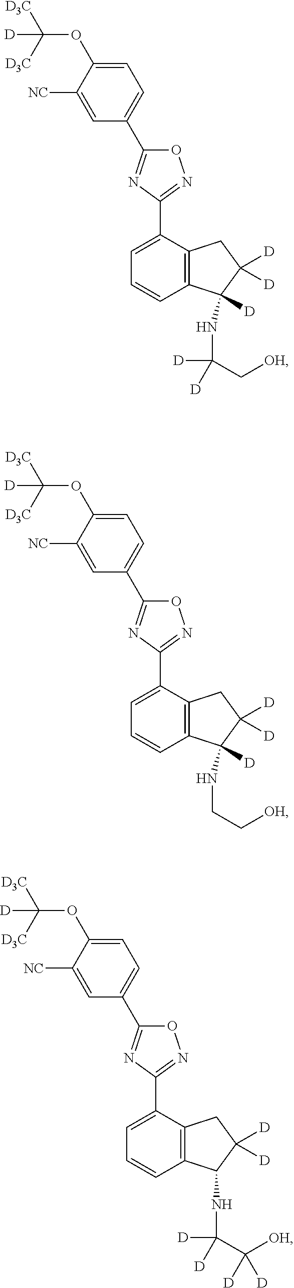 [SMILES: [2H]C([2H])(CO)N[C@]1([2H])c2cccc(-c3noc(-c4ccc(OC([2H])(C([2H])([2H])[2H])C([2H])([2H])[2H])c(C#N)c4)n3)c2CC1([2H])[2H] ; [2H]C([2H])([2H])C([2H])(Oc1ccc(-c2nc(-c3cccc4c3CC([2H])([2H])[C@]4([2H])NCCO)no2)cc1C#N)C([2H])([2H])[2H] ; [2H]C1([2H])Cc2c(-c3noc(-c4ccc(OC([2H])(C([2H])([2H])[2H])C([2H])([2H])[2H])c(C#N)c4)n3)cccc2[C@H]1NC([2H])([2H])C([2H])([2H])O] 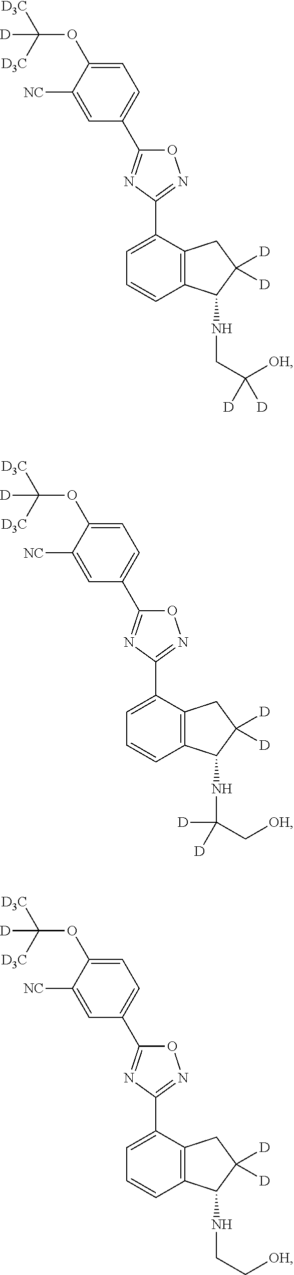 [SMILES: [2H]C([2H])(O)CN[C@@H]1c2cccc(-c3noc(-c4ccc(OC([2H])(C([2H])([2H])[2H])C([2H])([2H])[2H])c(C#N)c4)n3)c2CC1([2H])[2H] ; [2H]C([2H])(CO)N[C@@H]1c2cccc(-c3noc(-c4ccc(OC([2H])(C([2H])([2H])[2H])C([2H])([2H])[2H])c(C#N)c4)n3)c2CC1([2H])[2H] ; [2H]C1([2H])Cc2c(-c3noc(-c4ccc(OC([2H])(C([2H])([2H])[2H])C([2H])([2H])[2H])c(C#N)c4)n3)cccc2[C@H]1NCCO] 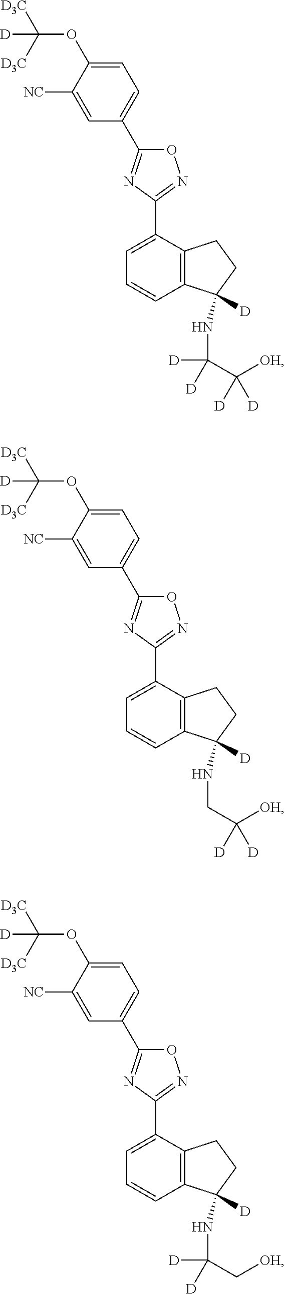 [SMILES: [2H]C([2H])([2H])C([2H])(Oc1ccc(-c2nc(-c3cccc4c3CC[C@]4([2H])NC([2H])([2H])C([2H])([2H])O)no2)cc1C#N)C([2H])([2H])[2H] ; [2H]C([2H])(O)CN[C@@]1([2H])CCc2c(-c3noc(-c4ccc(OC([2H])(C([2H])([2H])[2H])C([2H])([2H])[2H])c(C#N)c4)n3)cccc21 ; [2H]C([2H])(CO)N[C@@]1([2H])CCc2c(-c3noc(-c4ccc(OC([2H])(C([2H])([2H])[2H])C([2H])([2H])[2H])c(C#N)c4)n3)cccc21] 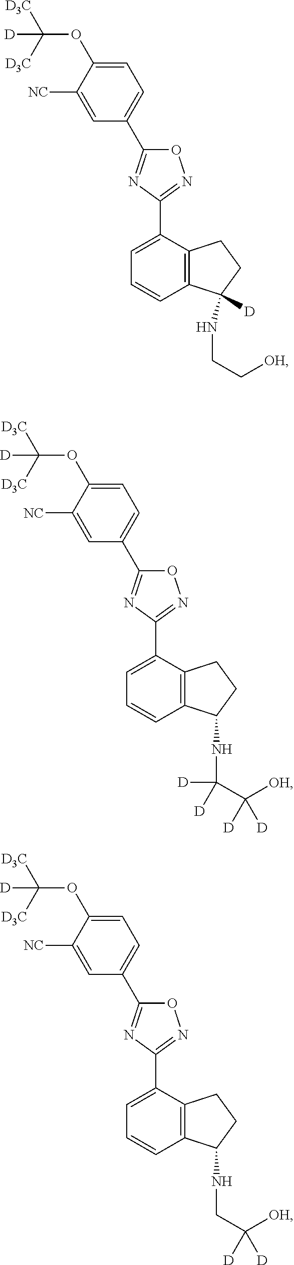 [SMILES: [2H]C([2H])([2H])C([2H])(Oc1ccc(-c2nc(-c3cccc4c3CC[C@]4([2H])NCCO)no2)cc1C#N)C([2H])([2H])[2H] ; [2H]C([2H])([2H])C([2H])(Oc1ccc(-c2nc(-c3cccc4c3CC[C@@H]4NC([2H])([2H])C([2H])([2H])O)no2)cc1C#N)C([2H])([2H])[2H] ; [2H]C([2H])(O)CN[C@H]1CCc2c(-c3noc(-c4ccc(OC([2H])(C([2H])([2H])[2H])C([2H])([2H])[2H])c(C#N)c4)n3)cccc21] 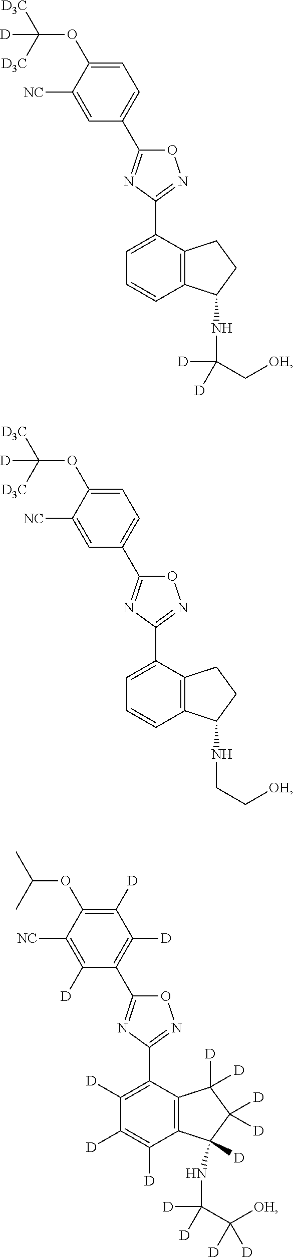 [SMILES: [2H]C([2H])(CO)N[C@H]1CCc2c(-c3noc(-c4ccc(OC([2H])(C([2H])([2H])[2H])C([2H])([2H])[2H])c(C#N)c4)n3)cccc21 ; [2H]C([2H])([2H])C([2H])(Oc1ccc(-c2nc(-c3cccc4c3CC[C@@H]4NCCO)no2)cc1C#N)C([2H])([2H])[2H] ; [2H]c1c([2H])c(-c2noc(-c3c([2H])c([2H])c(OC(C)C)c(C#N)c3[2H])n2)c2c(c1[2H])[C@@]([2H])(NC([2H])([2H])C([2H])([2H])O)C([2H])([2H])C2([2H])[2H]] 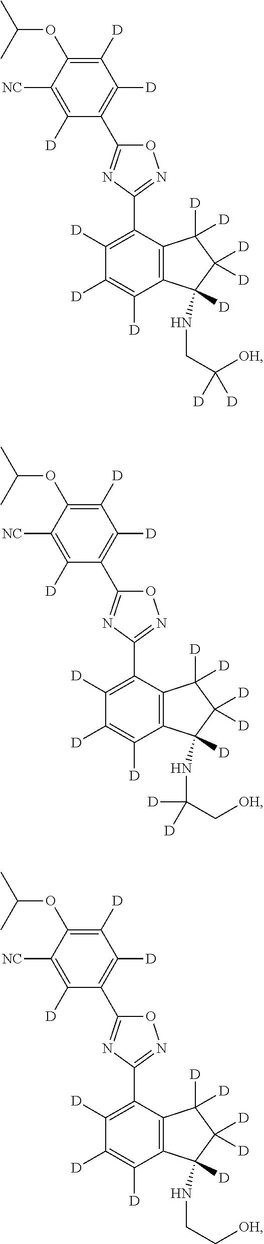 [SMILES: [2H]c1c([2H])c(-c2noc(-c3c([2H])c([2H])c(OC(C)C)c(C#N)c3[2H])n2)c2c(c1[2H])[C@@]([2H])(NCC([2H])([2H])O)C([2H])([2H])C2([2H])[2H] ; [2H]c1c([2H])c(-c2noc(-c3c([2H])c([2H])c(OC(C)C)c(C#N)c3[2H])n2)c2c(c1[2H])[C@@]([2H])(NC([2H])([2H])CO)C([2H])([2H])C2([2H])[2H] ; [2H]c1c([2H])c(-c2noc(-c3c([2H])c([2H])c(OC(C)C)c(C#N)c3[2H])n2)c2c(c1[2H])[C@@]([2H])(NCCO)C([2H])([2H])C2([2H])[2H]] 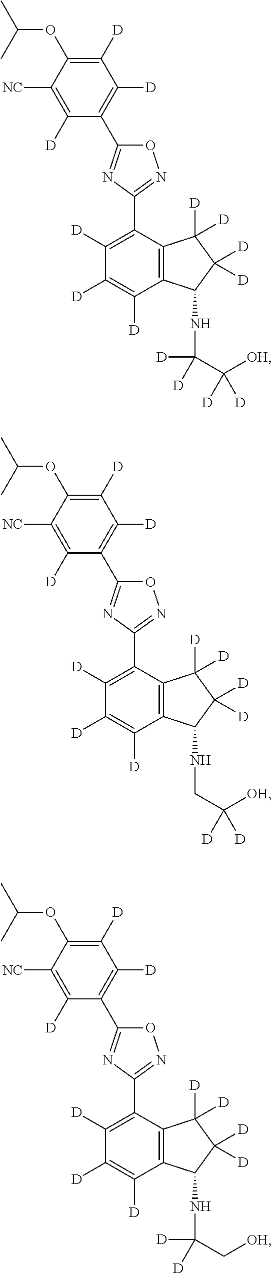 [SMILES: [2H]c1c([2H])c(-c2noc(-c3c([2H])c([2H])c(OC(C)C)c(C#N)c3[2H])n2)c2c(c1[2H])[C@@H](NC([2H])([2H])C([2H])([2H])O)C([2H])([2H])C2([2H])[2H] ; [2H]c1c([2H])c(-c2noc(-c3c([2H])c([2H])c(OC(C)C)c(C#N)c3[2H])n2)c2c(c1[2H])[C@@H](NCC([2H])([2H])O)C([2H])([2H])C2([2H])[2H] ; [2H]c1c([2H])c(-c2noc(-c3c([2H])c([2H])c(OC(C)C)c(C#N)c3[2H])n2)c2c(c1[2H])[C@@H](NC([2H])([2H])CO)C([2H])([2H])C2([2H])[2H]] 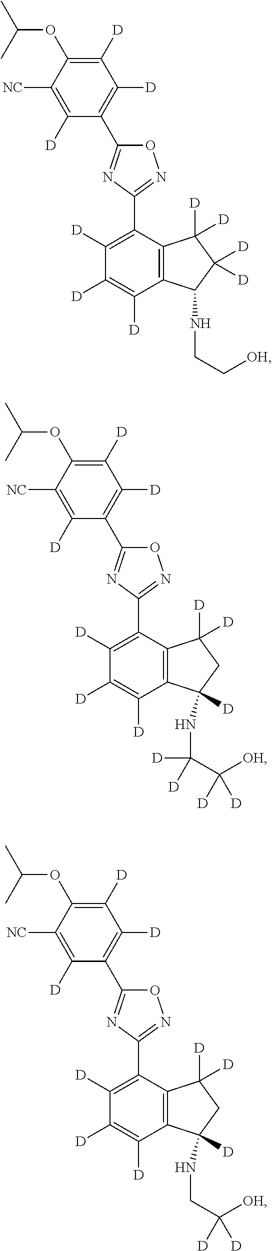 [SMILES: [2H]c1c([2H])c(-c2noc(-c3c([2H])c([2H])c(OC(C)C)c(C#N)c3[2H])n2)c2c(c1[2H])[C@@H](NCCO)C([2H])([2H])C2([2H])[2H] ; [2H]c1c([2H])c(-c2noc(-c3c([2H])c([2H])c(OC(C)C)c(C#N)c3[2H])n2)c2c(c1[2H])[C@@]([2H])(NC([2H])([2H])C([2H])([2H])O)CC2([2H])[2H] ; [2H]c1c([2H])c(-c2noc(-c3c([2H])c([2H])c(OC(C)C)c(C#N)c3[2H])n2)c2c(c1[2H])[C@@]([2H])(NCC([2H])([2H])O)CC2([2H])[2H]] 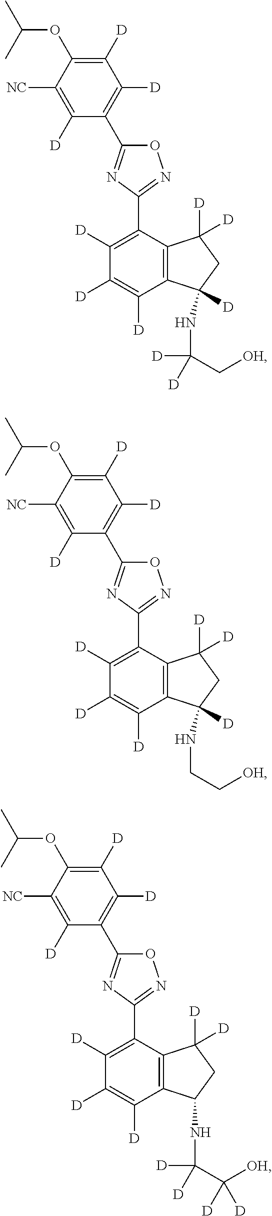 [SMILES: [2H]c1c([2H])c(-c2noc(-c3c([2H])c([2H])c(OC(C)C)c(C#N)c3[2H])n2)c2c(c1[2H])[C@@]([2H])(NC([2H])([2H])CO)CC2([2H])[2H] ; [2H]c1c([2H])c(-c2noc(-c3c([2H])c([2H])c(OC(C)C)c(C#N)c3[2H])n2)c2c(c1[2H])[C@@]([2H])(NCCO)CC2([2H])[2H] ; [2H]c1c([2H])c(-c2noc(-c3c([2H])c([2H])c(OC(C)C)c(C#N)c3[2H])n2)c2c(c1[2H])[C@@H](NC([2H])([2H])C([2H])([2H])O)CC2([2H])[2H]] 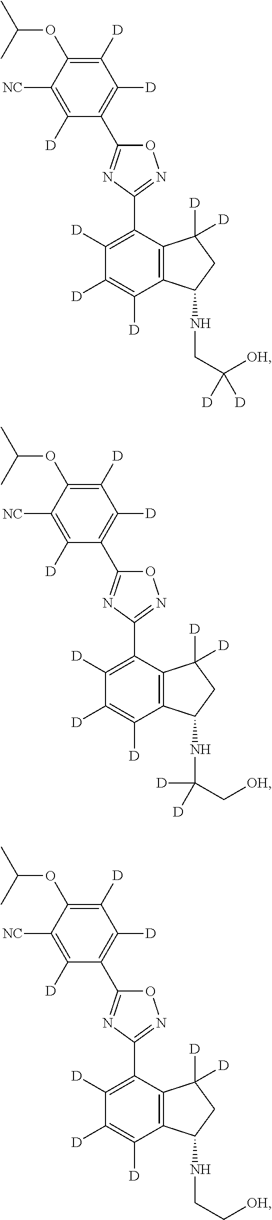 [SMILES: [2H]c1c([2H])c(-c2noc(-c3c([2H])c([2H])c(OC(C)C)c(C#N)c3[2H])n2)c2c(c1[2H])[C@@H](NCC([2H])([2H])O)CC2([2H])[2H] ; [2H]c1c([2H])c(-c2noc(-c3c([2H])c([2H])c(OC(C)C)c(C#N)c3[2H])n2)c2c(c1[2H])[C@@H](NC([2H])([2H])CO)CC2([2H])[2H] ; [2H]c1c([2H])c(-c2noc(-c3c([2H])c([2H])c(OC(C)C)c(C#N)c3[2H])n2)c2c(c1[2H])[C@@H](NCCO)CC2([2H])[2H]] 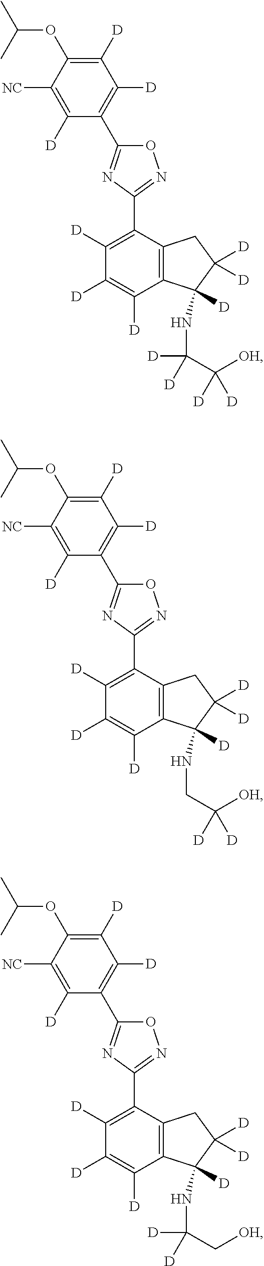 [SMILES: [2H]c1c([2H])c(-c2noc(-c3c([2H])c([2H])c(OC(C)C)c(C#N)c3[2H])n2)c2c(c1[2H])[C@@]([2H])(NC([2H])([2H])C([2H])([2H])O)C([2H])([2H])C2 ; [2H]c1c([2H])c(-c2noc(-c3c([2H])c([2H])c(OC(C)C)c(C#N)c3[2H])n2)c2c(c1[2H])[C@@]([2H])(NCC([2H])([2H])O)C([2H])([2H])C2 ; [2H]c1c([2H])c(-c2noc(-c3c([2H])c([2H])c(OC(C)C)c(C#N)c3[2H])n2)c2c(c1[2H])[C@@]([2H])(NC([2H])([2H])CO)C([2H])([2H])C2] 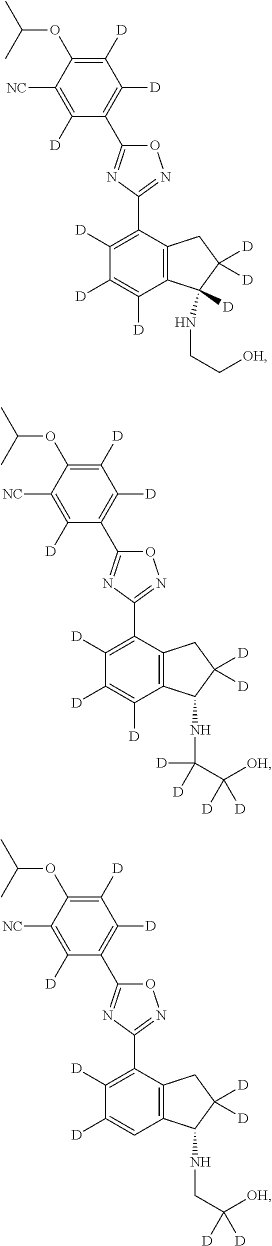 [SMILES: [2H]c1c([2H])c(-c2noc(-c3c([2H])c([2H])c(OC(C)C)c(C#N)c3[2H])n2)c2c(c1[2H])[C@@]([2H])(NCCO)C([2H])([2H])C2 ; [2H]c1c([2H])c(-c2noc(-c3c([2H])c([2H])c(OC(C)C)c(C#N)c3[2H])n2)c2c(c1[2H])[C@@H](NC([2H])([2H])C([2H])([2H])O)C([2H])([2H])C2 ; [2H]c1cc2c(c(-c3noc(-c4c([2H])c([2H])c(OC(C)C)c(C#N)c4[2H])n3)c1[2H])CC([2H])([2H])[C@@H]2NCC([2H])([2H])O] 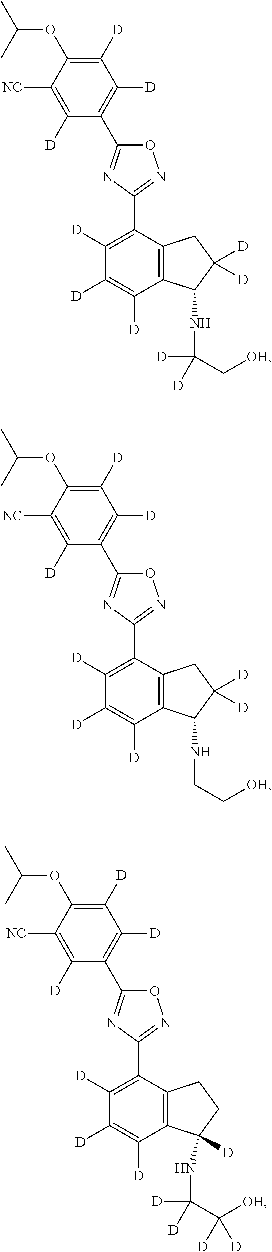 [SMILES: [2H]c1c([2H])c(-c2noc(-c3c([2H])c([2H])c(OC(C)C)c(C#N)c3[2H])n2)c2c(c1[2H])[C@@H](NC([2H])([2H])CO)C([2H])([2H])C2 ; [2H]c1c([2H])c(-c2noc(-c3c([2H])c([2H])c(OC(C)C)c(C#N)c3[2H])n2)c2c(c1[2H])[C@@H](NCCO)C([2H])([2H])C2 ; [2H]c1c([2H])c(-c2noc(-c3c([2H])c([2H])c(OC(C)C)c(C#N)c3[2H])n2)c2c(c1[2H])[C@@]([2H])(NC([2H])([2H])C([2H])([2H])O)CC2] 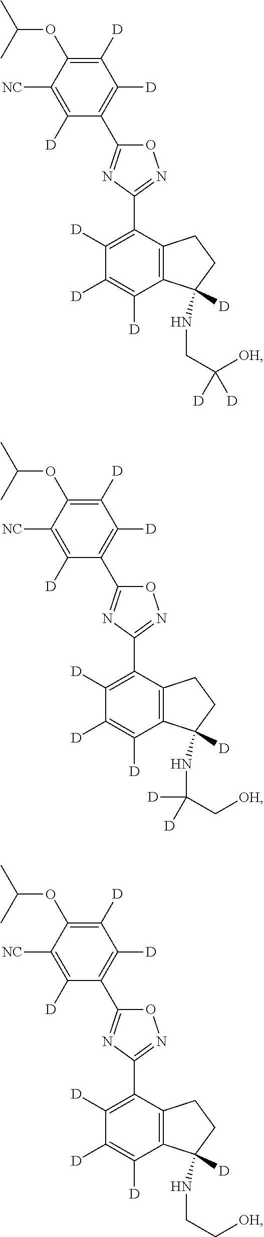 [SMILES: [2H]c1c([2H])c(-c2noc(-c3c([2H])c([2H])c(OC(C)C)c(C#N)c3[2H])n2)c2c(c1[2H])[C@@]([2H])(NCC([2H])([2H])O)CC2 ; [2H]c1c([2H])c(-c2noc(-c3c([2H])c([2H])c(OC(C)C)c(C#N)c3[2H])n2)c2c(c1[2H])[C@@]([2H])(NC([2H])([2H])CO)CC2 ; [2H]c1c([2H])c(-c2noc(-c3c([2H])c([2H])c(OC(C)C)c(C#N)c3[2H])n2)c2c(c1[2H])[C@@]([2H])(NCCO)CC2] 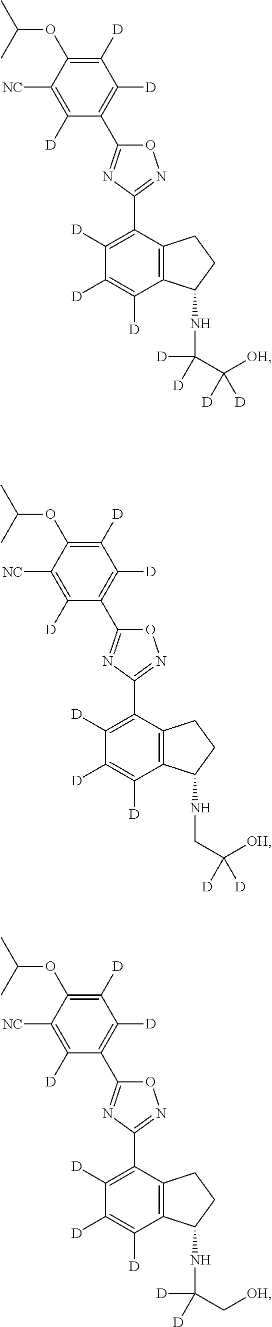 [SMILES: [2H]c1c([2H])c(-c2noc(-c3c([2H])c([2H])c(OC(C)C)c(C#N)c3[2H])n2)c2c(c1[2H])[C@@H](NC([2H])([2H])C([2H])([2H])O)CC2 ; [2H]c1c([2H])c(-c2noc(-c3c([2H])c([2H])c(OC(C)C)c(C#N)c3[2H])n2)c2c(c1[2H])[C@@H](NCC([2H])([2H])O)CC2 ; [2H]c1c([2H])c(-c2noc(-c3c([2H])c([2H])c(OC(C)C)c(C#N)c3[2H])n2)c2c(c1[2H])[C@@H](NC([2H])([2H])CO)CC2] 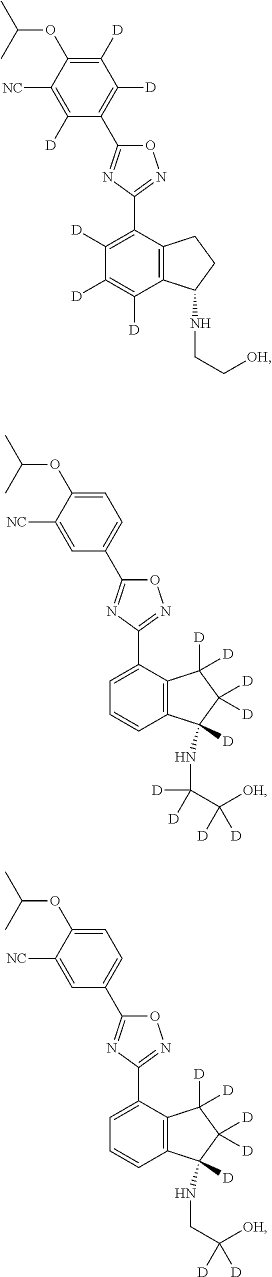 [SMILES: [2H]c1c([2H])c(-c2noc(-c3c([2H])c([2H])c(OC(C)C)c(C#N)c3[2H])n2)c2c(c1[2H])[C@@H](NCCO)CC2 ; [2H]C([2H])(O)C([2H])([2H])N[C@]1([2H])c2cccc(-c3noc(-c4ccc(OC(C)C)c(C#N)c4)n3)c2C([2H])([2H])C1([2H])[2H] ; [2H]C([2H])(O)CN[C@]1([2H])c2cccc(-c3noc(-c4ccc(OC(C)C)c(C#N)c4)n3)c2C([2H])([2H])C1([2H])[2H]] 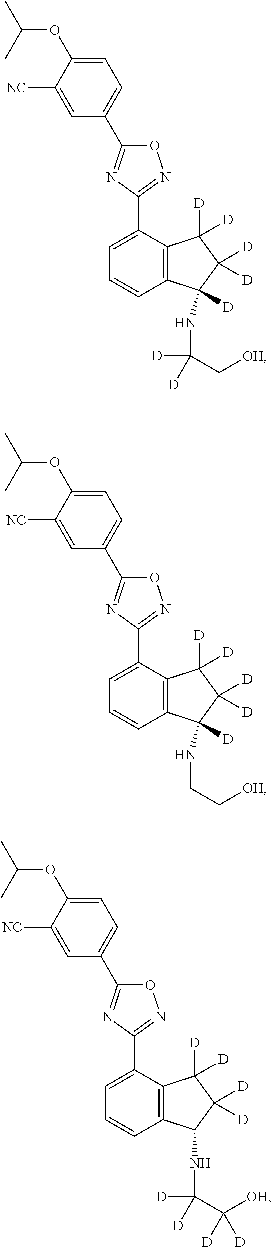 [SMILES: [2H]C([2H])(CO)N[C@]1([2H])c2cccc(-c3noc(-c4ccc(OC(C)C)c(C#N)c4)n3)c2C([2H])([2H])C1([2H])[2H] ; [2H]C1([2H])c2c(-c3noc(-c4ccc(OC(C)C)c(C#N)c4)n3)cccc2[C@@]([2H])(NCCO)C1([2H])[2H] ; [2H]C([2H])(O)C([2H])([2H])N[C@@H]1c2cccc(-c3noc(-c4ccc(OC(C)C)c(C#N)c4)n3)c2C([2H])([2H])C1([2H])[2H]] 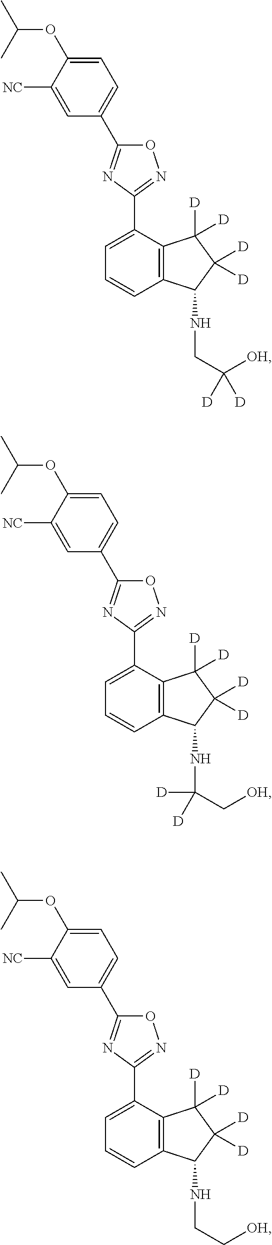 [SMILES: [2H]C([2H])(O)CN[C@@H]1c2cccc(-c3noc(-c4ccc(OC(C)C)c(C#N)c4)n3)c2C([2H])([2H])C1([2H])[2H] ; [2H]C([2H])(CO)N[C@@H]1c2cccc(-c3noc(-c4ccc(OC(C)C)c(C#N)c4)n3)c2C([2H])([2H])C1([2H])[2H] ; [2H]C1([2H])c2c(-c3noc(-c4ccc(OC(C)C)c(C#N)c4)n3)cccc2[C@@H](NCCO)C1([2H])[2H]] 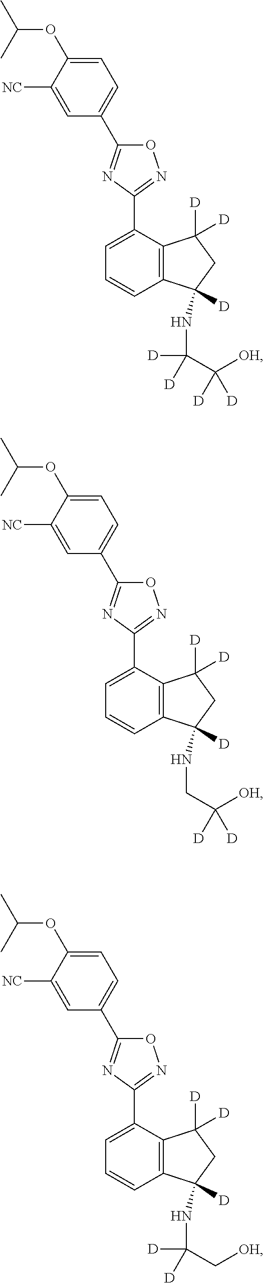 [SMILES: [2H]C1([2H])C[C@]([2H])(NC([2H])([2H])C([2H])([2H])O)c2cccc(-c3noc(-c4ccc(OC(C)C)c(C#N)c4)n3)c21 ; [2H]C([2H])(O)CN[C@@]1([2H])CC([2H])([2H])c2c(-c3noc(-c4ccc(OC(C)C)c(C#N)c4)n3)cccc21 ; [2H]C([2H])(CO)N[C@@]1([2H])CC([2H])([2H])c2c(-c3noc(-c4ccc(OC(C)C)c(C#N)c4)n3)cccc21] 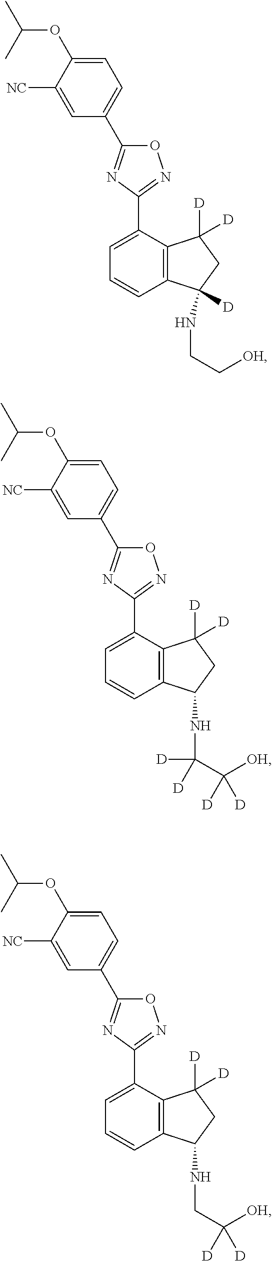 [SMILES: [2H]C1([2H])C[C@]([2H])(NCCO)c2cccc(-c3noc(-c4ccc(OC(C)C)c(C#N)c4)n3)c21 ; [2H]C1([2H])C[C@H](NC([2H])([2H])C([2H])([2H])O)c2cccc(-c3noc(-c4ccc(OC(C)C)c(C#N)c4)n3)c21 ; [2H]C([2H])(O)CN[C@H]1CC([2H])([2H])c2c(-c3noc(-c4ccc(OC(C)C)c(C#N)c4)n3)cccc21] 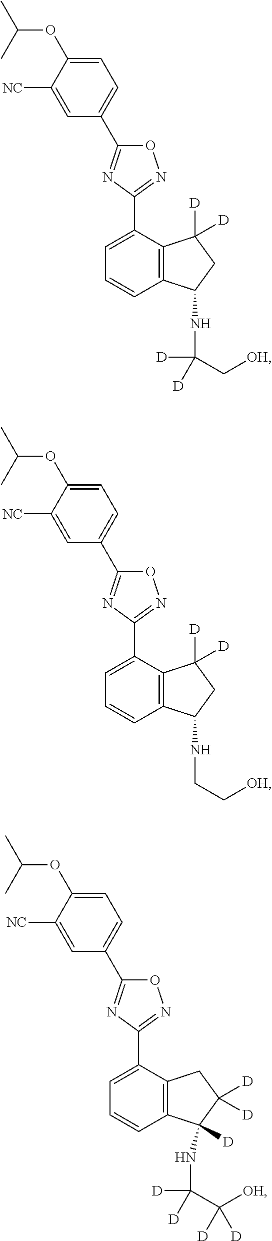 [SMILES: [2H]C([2H])(CO)N[C@H]1CC([2H])([2H])c2c(-c3noc(-c4ccc(OC(C)C)c(C#N)c4)n3)cccc21 ; [2H]C1([2H])C[C@H](NCCO)c2cccc(-c3noc(-c4ccc(OC(C)C)c(C#N)c4)n3)c21 ; [2H]C([2H])(O)C([2H])([2H])N[C@]1([2H])c2cccc(-c3noc(-c4ccc(OC(C)C)c(C#N)c4)n3)c2CC1([2H])[2H]] 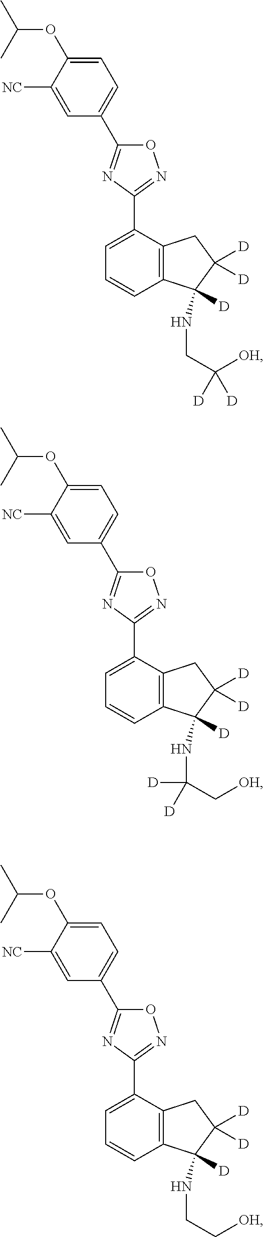 [SMILES: [2H]C([2H])(O)CN[C@]1([2H])c2cccc(-c3noc(-c4ccc(OC(C)C)c(C#N)c4)n3)c2CC1([2H])[2H] ; [2H]C([2H])(CO)N[C@]1([2H])c2cccc(-c3noc(-c4ccc(OC(C)C)c(C#N)c4)n3)c2CC1([2H])[2H] ; [2H]C1([2H])Cc2c(-c3noc(-c4ccc(OC(C)C)c(C#N)c4)n3)cccc2[C@@]1([2H])NCCO] 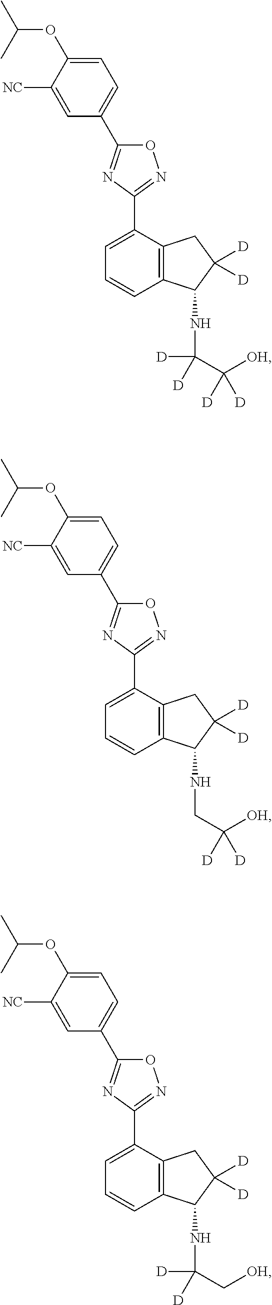 [SMILES: [2H]C1([2H])Cc2c(-c3noc(-c4ccc(OC(C)C)c(C#N)c4)n3)cccc2[C@H]1NC([2H])([2H])C([2H])([2H])O ; [2H]C([2H])(O)CN[C@@H]1c2cccc(-c3noc(-c4ccc(OC(C)C)c(C#N)c4)n3)c2CC1([2H])[2H] ; [2H]C([2H])(CO)N[C@@H]1c2cccc(-c3noc(-c4ccc(OC(C)C)c(C#N)c4)n3)c2CC1([2H])[2H]] 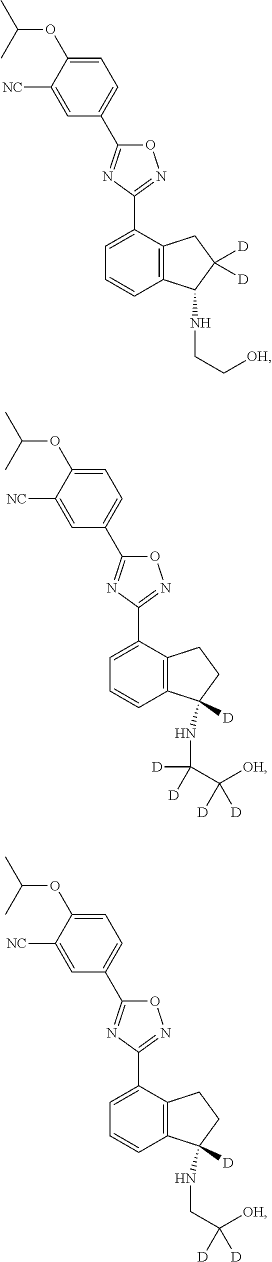 [SMILES: [2H]C1([2H])Cc2c(-c3noc(-c4ccc(OC(C)C)c(C#N)c4)n3)cccc2[C@H]1NCCO ; [2H]C([2H])(O)C([2H])([2H])N[C@@]1([2H])CCc2c(-c3noc(-c4ccc(OC(C)C)c(C#N)c4)n3)cccc21 ; [2H]C([2H])(O)CN[C@@]1([2H])CCc2c(-c3noc(-c4ccc(OC(C)C)c(C#N)c4)n3)cccc21] 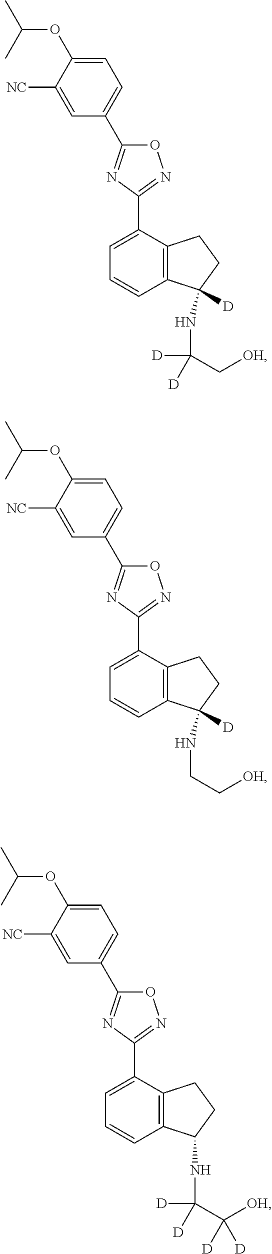 [SMILES: [2H]C([2H])(CO)N[C@@]1([2H])CCc2c(-c3noc(-c4ccc(OC(C)C)c(C#N)c4)n3)cccc21 ; [2H][C@]1(NCCO)CCc2c(-c3noc(-c4ccc(OC(C)C)c(C#N)c4)n3)cccc21 ; [2H]C([2H])(O)C([2H])([2H])N[C@H]1CCc2c(-c3noc(-c4ccc(OC(C)C)c(C#N)c4)n3)cccc21] 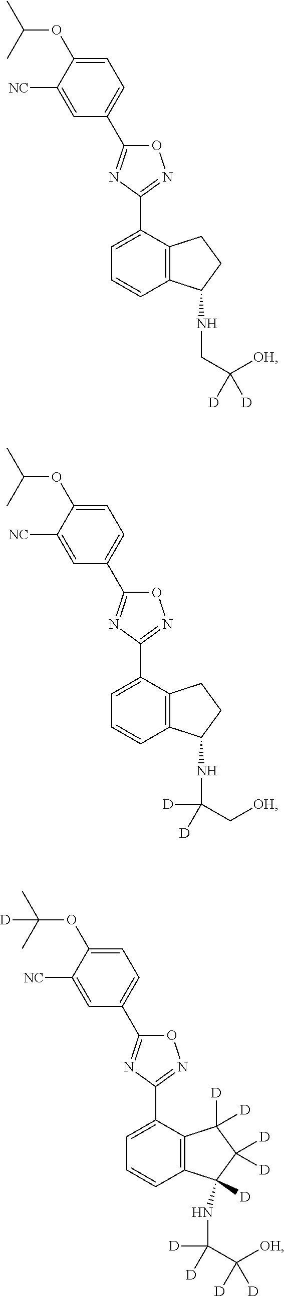 [SMILES: [2H]C([2H])(O)CN[C@H]1CCc2c(-c3noc(-c4ccc(OC(C)C)c(C#N)c4)n3)cccc21 ; [2H]C([2H])(CO)N[C@H]1CCc2c(-c3noc(-c4ccc(OC(C)C)c(C#N)c4)n3)cccc21 ; [2H]C(C)(C)Oc1ccc(-c2nc(-c3cccc4c3C([2H])([2H])C([2H])([2H])[C@]4([2H])NC([2H])([2H])C([2H])([2H])O)no2)cc1C#N] 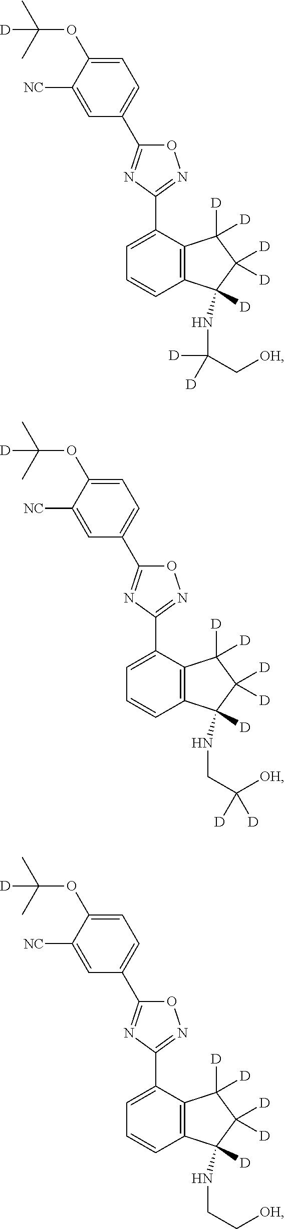 [SMILES: [2H]C([2H])(CO)N[C@]1([2H])c2cccc(-c3noc(-c4ccc(OC([2H])(C)C)c(C#N)c4)n3)c2C([2H])([2H])C1([2H])[2H] ; [2H]C([2H])(O)CN[C@]1([2H])c2cccc(-c3noc(-c4ccc(OC([2H])(C)C)c(C#N)c4)n3)c2C([2H])([2H])C1([2H])[2H] ; [2H]C(C)(C)Oc1ccc(-c2nc(-c3cccc4c3C([2H])([2H])C([2H])([2H])[C@]4([2H])NCCO)no2)cc1C#N] 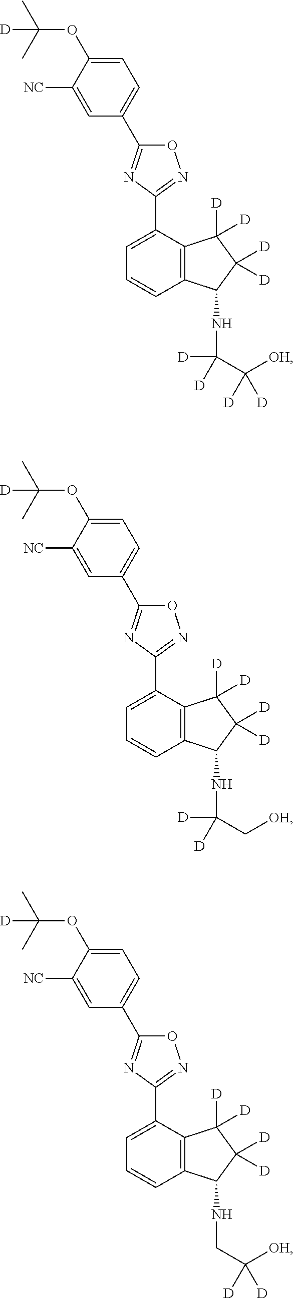 [SMILES: [2H]C(C)(C)Oc1ccc(-c2nc(-c3cccc4c3C([2H])([2H])C([2H])([2H])[C@@H]4NC([2H])([2H])C([2H])([2H])O)no2)cc1C#N ; [2H]C([2H])(CO)N[C@@H]1c2cccc(-c3noc(-c4ccc(OC([2H])(C)C)c(C#N)c4)n3)c2C([2H])([2H])C1([2H])[2H] ; [2H]C([2H])(O)CN[C@@H]1c2cccc(-c3noc(-c4ccc(OC([2H])(C)C)c(C#N)c4)n3)c2C([2H])([2H])C1([2H])[2H]] 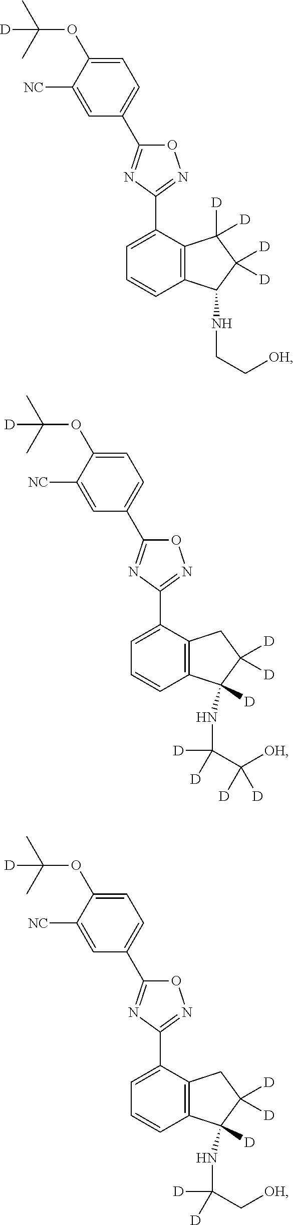 [SMILES: [2H]C(C)(C)Oc1ccc(-c2nc(-c3cccc4c3C([2H])([2H])C([2H])([2H])[C@@H]4NCCO)no2)cc1C#N ; [2H]C(C)(C)Oc1ccc(-c2nc(-c3cccc4c3CC([2H])([2H])[C@]4([2H])NC([2H])([2H])C([2H])([2H])O)no2)cc1C#N ; [2H]C([2H])(CO)N[C@]1([2H])c2cccc(-c3noc(-c4ccc(OC([2H])(C)C)c(C#N)c4)n3)c2CC1([2H])[2H]] 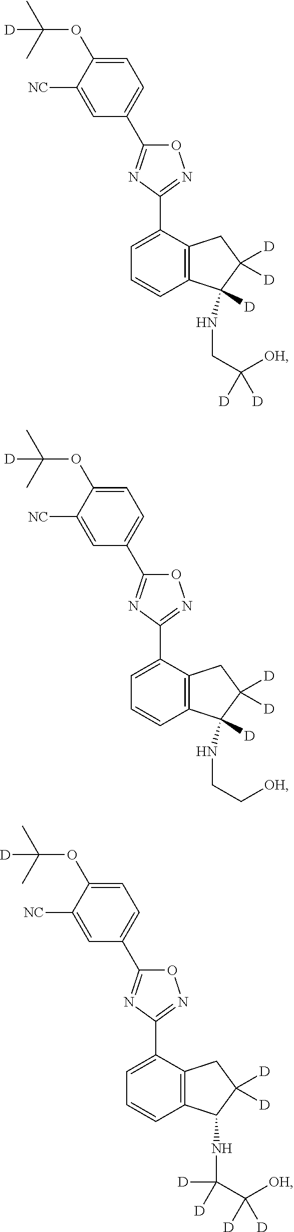 [SMILES: [2H]C([2H])(O)CN[C@]1([2H])c2cccc(-c3noc(-c4ccc(OC([2H])(C)C)c(C#N)c4)n3)c2CC1([2H])[2H] ; [2H]C(C)(C)Oc1ccc(-c2nc(-c3cccc4c3CC([2H])([2H])[C@]4([2H])NCCO)no2)cc1C#N ; [2H]C(C)(C)Oc1ccc(-c2nc(-c3cccc4c3CC([2H])([2H])[C@@H]4NC([2H])([2H])C([2H])([2H])O)no2)cc1C#N] 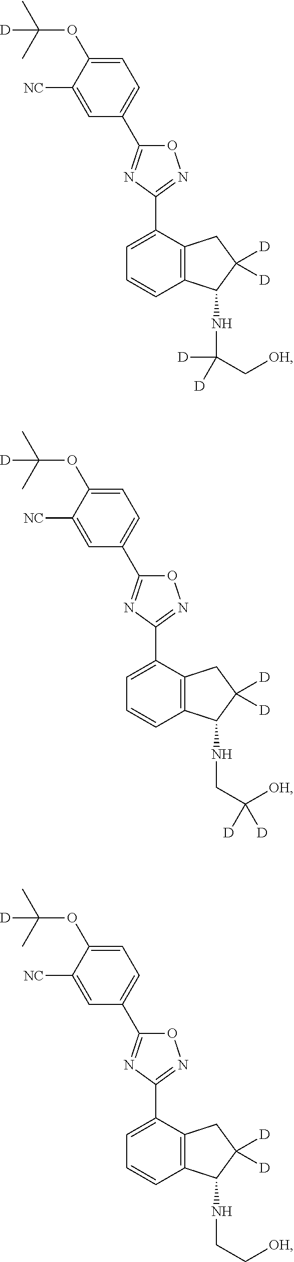 [SMILES: [2H]C([2H])(CO)N[C@@H]1c2cccc(-c3noc(-c4ccc(OC([2H])(C)C)c(C#N)c4)n3)c2CC1([2H])[2H] ; [2H]C([2H])(O)CN[C@@H]1c2cccc(-c3noc(-c4ccc(OC([2H])(C)C)c(C#N)c4)n3)c2CC1([2H])[2H] ; [2H]C(C)(C)Oc1ccc(-c2nc(-c3cccc4c3CC([2H])([2H])[C@@H]4NCCO)no2)cc1C#N] 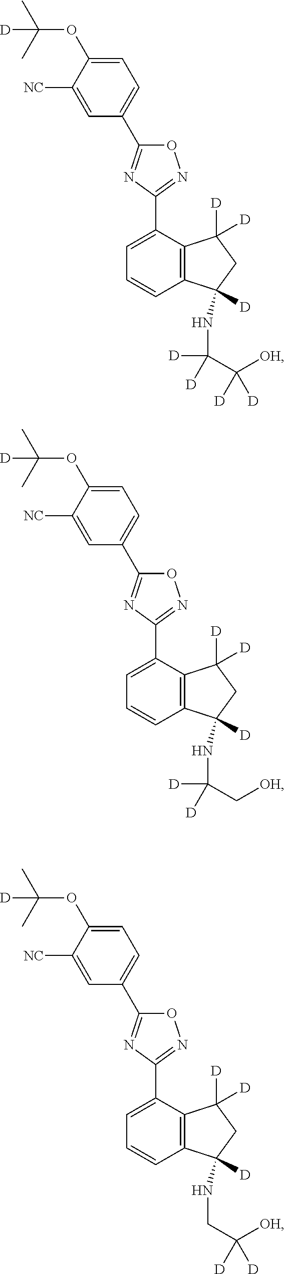 [SMILES: [2H]C(C)(C)Oc1ccc(-c2nc(-c3cccc4c3C([2H])([2H])C[C@]4([2H])NC([2H])([2H])C([2H])([2H])O)no2)cc1C#N ; [2H]C([2H])(CO)N[C@@]1([2H])CC([2H])([2H])c2c(-c3noc(-c4ccc(OC([2H])(C)C)c(C#N)c4)n3)cccc21 ; [2H]C([2H])(O)CN[C@@]1([2H])CC([2H])([2H])c2c(-c3noc(-c4ccc(OC([2H])(C)C)c(C#N)c4)n3)cccc21] 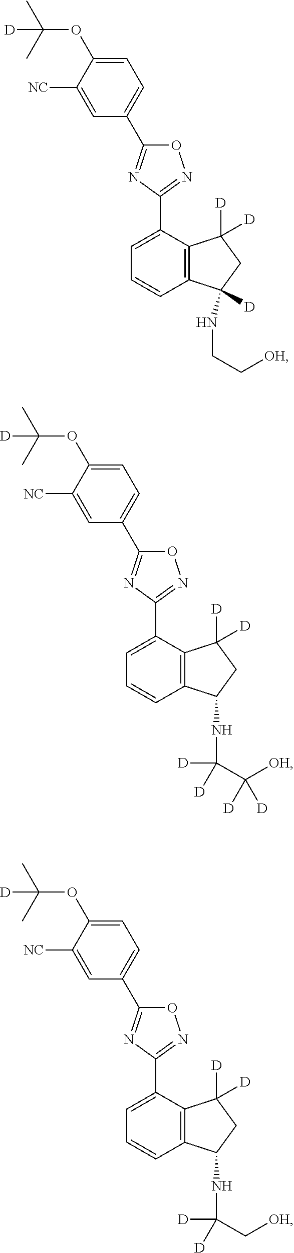 [SMILES: [2H]C(C)(C)Oc1ccc(-c2nc(-c3cccc4c3C([2H])([2H])C[C@]4([2H])NCCO)no2)cc1C#N ; [2H]C(C)(C)Oc1ccc(-c2nc(-c3cccc4c3C([2H])([2H])C[C@@H]4NC([2H])([2H])C([2H])([2H])O)no2)cc1C#N ; [2H]C([2H])(CO)N[C@H]1CC([2H])([2H])c2c(-c3noc(-c4ccc(OC([2H])(C)C)c(C#N)c4)n3)cccc21] 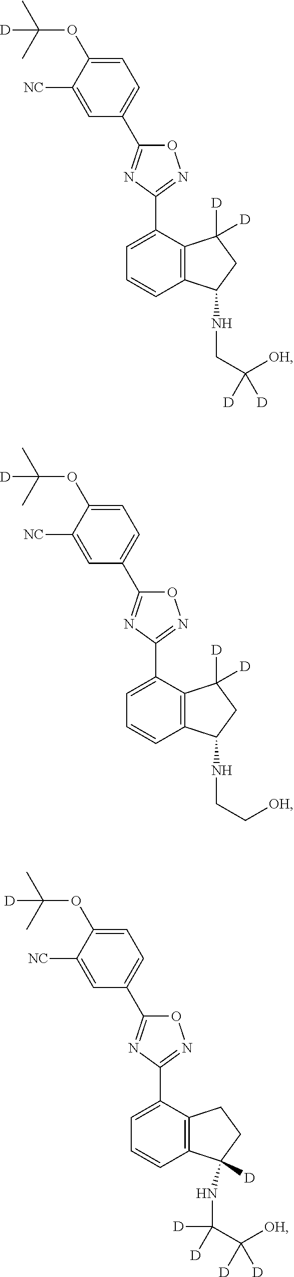 [SMILES: [2H]C([2H])(O)CN[C@H]1CC([2H])([2H])c2c(-c3noc(-c4ccc(OC([2H])(C)C)c(C#N)c4)n3)cccc21 ; [2H]C(C)(C)Oc1ccc(-c2nc(-c3cccc4c3C([2H])([2H])C[C@@H]4NCCO)no2)cc1C#N ; [2H]C(C)(C)Oc1ccc(-c2nc(-c3cccc4c3CC[C@]4([2H])NC([2H])([2H])C([2H])([2H])O)no2)cc1C#N] 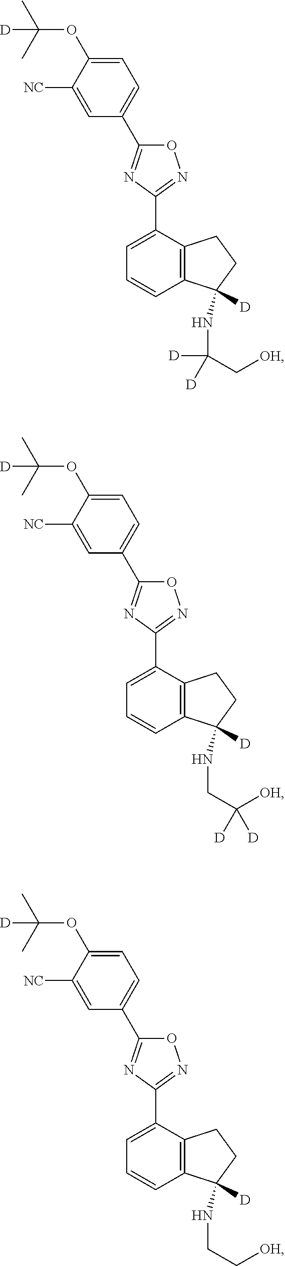 [SMILES: [2H]C([2H])(CO)N[C@@]1([2H])CCc2c(-c3noc(-c4ccc(OC([2H])(C)C)c(C#N)c4)n3)cccc21 ; [2H]C([2H])(O)CN[C@@]1([2H])CCc2c(-c3noc(-c4ccc(OC([2H])(C)C)c(C#N)c4)n3)cccc21 ; [2H]C(C)(C)Oc1ccc(-c2nc(-c3cccc4c3CC[C@]4([2H])NCCO)no2)cc1C#N] 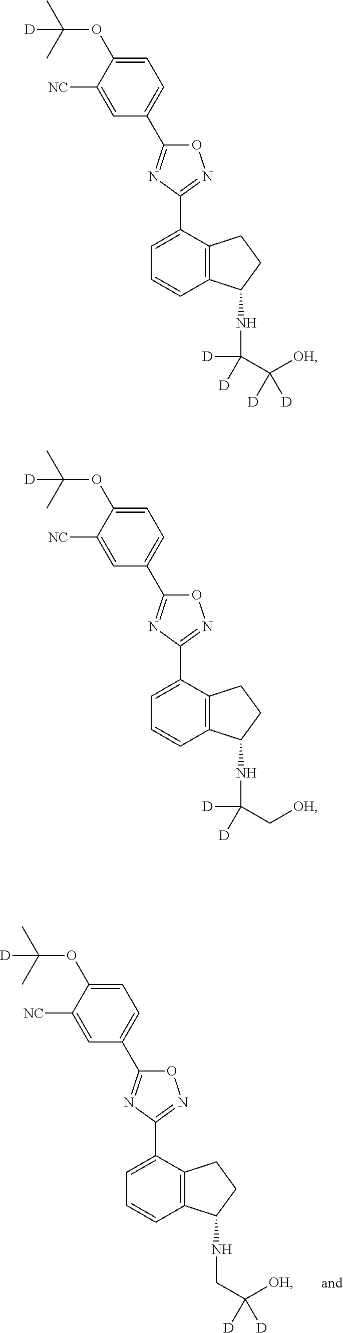 [SMILES: [2H]C(C)(C)Oc1ccc(-c2nc(-c3cccc4c3CC[C@@H]4NC([2H])([2H])C([2H])([2H])O)no2)cc1C#N ; [2H]C([2H])(CO)N[C@H]1CCc2c(-c3noc(-c4ccc(OC([2H])(C)C)c(C#N)c4)n3)cccc21 ; [2H]C([2H])(O)CN[C@H]1CCc2c(-c3noc(-c4ccc(OC([2H])(C)C)c(C#N)c4)n3)cccc21 ; "and"] 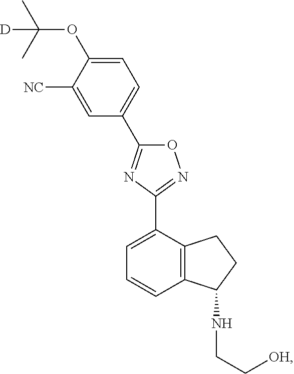 [SMILES: [2H]C(C)(C)Oc1ccc(-c2nc(-c3cccc4c3CC[C@@H]4NCCO)no2)cc1C#N]
or a salt thereof.

70. The compound as recited in claim 57 wherein said compound has a structural formula selected from the group consisting of 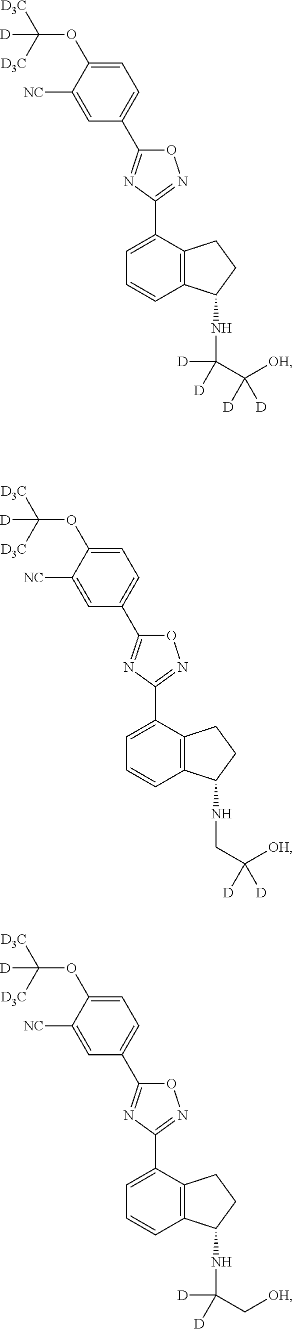 [SMILES: [2H]C([2H])([2H])C([2H])(Oc1ccc(-c2nc(-c3cccc4c3CC[C@@H]4NC([2H])([2H])C([2H])([2H])O)no2)cc1C#N)C([2H])([2H])[2H] ; [2H]C([2H])(O)CN[C@H]1CCc2c(-c3noc(-c4ccc(OC([2H])(C([2H])([2H])[2H])C([2H])([2H])[2H])c(C#N)c4)n3)cccc21 ; [2H]C([2H])(CO)N[C@H]1CCc2c(-c3noc(-c4ccc(OC([2H])(C([2H])([2H])[2H])C([2H])([2H])[2H])c(C#N)c4)n3)cccc21] 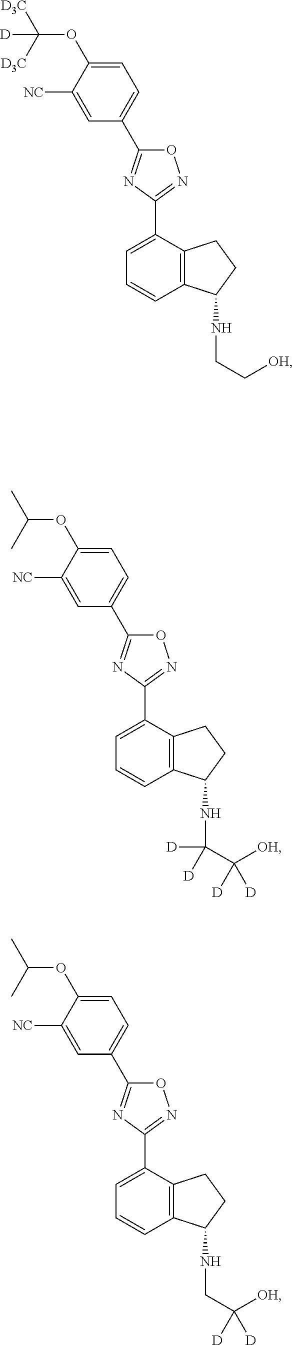 [SMILES: [2H]C([2H])([2H])C([2H])(Oc1ccc(-c2nc(-c3cccc4c3CC[C@@H]4NCCO)no2)cc1C#N)C([2H])([2H])[2H] ; [2H]C([2H])(O)C([2H])([2H])N[C@H]1CCc2c(-c3noc(-c4ccc(OC(C)C)c(C#N)c4)n3)cccc21 ; [2H]C([2H])(O)CN[C@H]1CCc2c(-c3noc(-c4ccc(OC(C)C)c(C#N)c4)n3)cccc21] 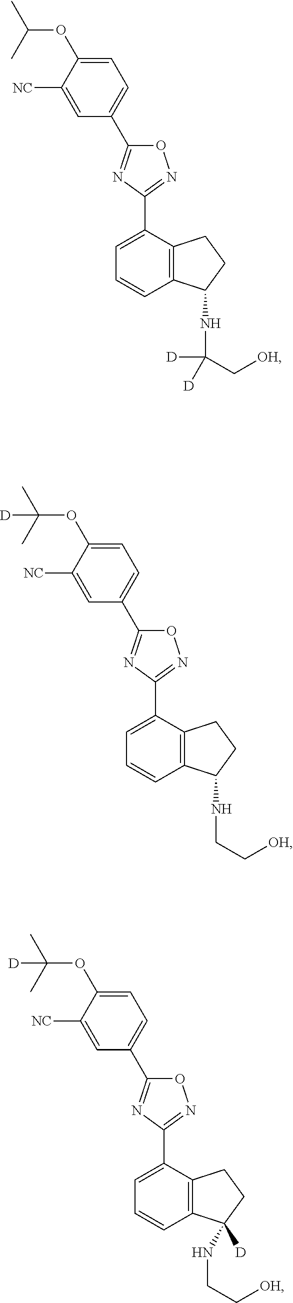 [SMILES: [2H]C([2H])(CO)N[C@H]1CCc2c(-c3noc(-c4ccc(OC(C)C)c(C#N)c4)n3)cccc21 ; [2H]C(C)(C)Oc1ccc(-c2nc(-c3cccc4c3CC[C@@H]4NCCO)no2)cc1C#N ; [2H]C(C)(C)Oc1ccc(-c2nc(-c3cccc4c3CC[C@]4([2H])NCCO)no2)cc1C#N] 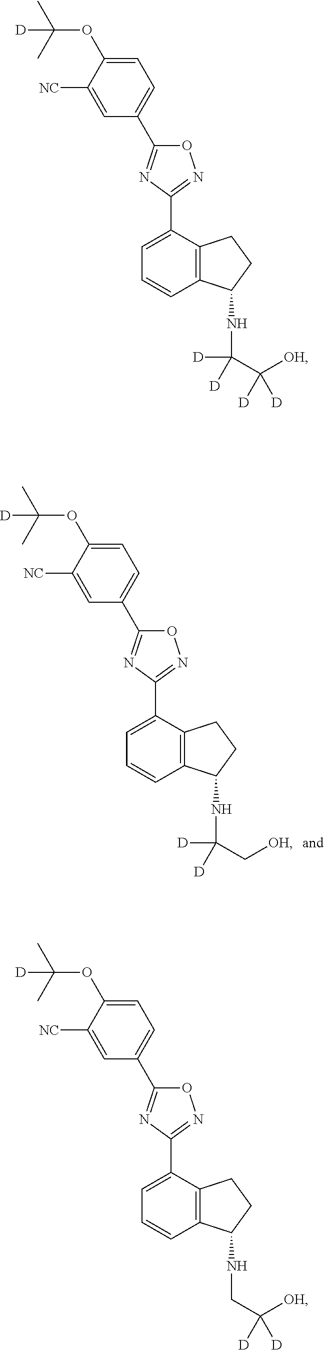 [SMILES: [2H]C(C)(C)Oc1ccc(-c2nc(-c3cccc4c3CC[C@@H]4NC([2H])([2H])C([2H])([2H])O)no2)cc1C#N ; [2H]C([2H])(CO)N[C@H]1CCc2c(-c3noc(-c4ccc(OC([2H])(C)C)c(C#N)c4)n3)cccc21 ; [2H]C([2H])(O)CN[C@H]1CCc2c(-c3noc(-c4ccc(OC([2H])(C)C)c(C#N)c4)n3)cccc21]
or a salt thereof.

71. The compound as recited in claim 57 wherein said compound has a structural formula selected from the group consisting of 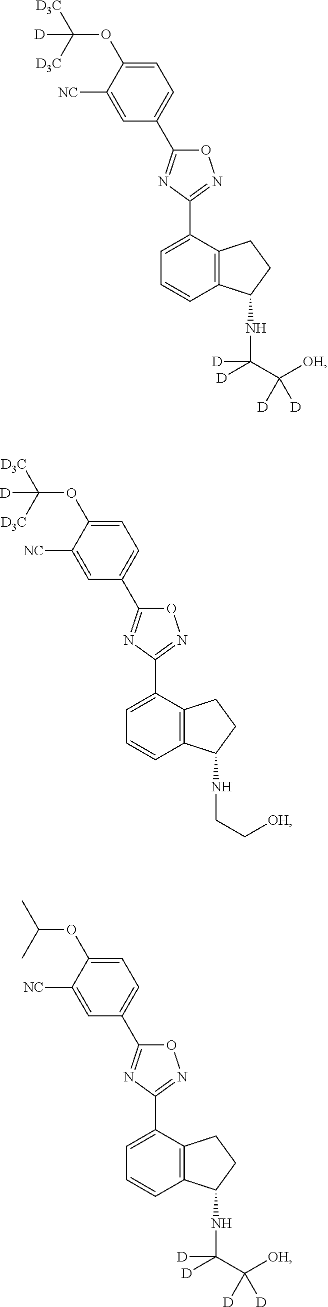 [SMILES: [2H]C([2H])([2H])C([2H])(Oc1ccc(-c2nc(-c3cccc4c3CC[C@@H]4NC([2H])([2H])C([2H])([2H])O)no2)cc1C#N)C([2H])([2H])[2H] ; [2H]C([2H])([2H])C([2H])(Oc1ccc(-c2nc(-c3cccc4c3CC[C@@H]4NCCO)no2)cc1C#N)C([2H])([2H])[2H] ; [2H]C([2H])(O)C([2H])([2H])N[C@H]1CCc2c(-c3noc(-c4ccc(OC(C)C)c(C#N)c4)n3)cccc21] 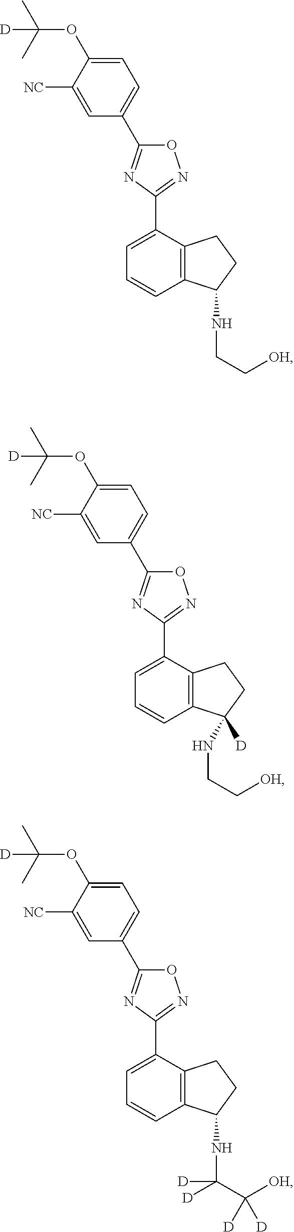 [SMILES: [2H]C(C)(C)Oc1ccc(-c2nc(-c3cccc4c3CC[C@@H]4NCCO)no2)cc1C#N ; [2H]C(C)(C)Oc1ccc(-c2nc(-c3cccc4c3CC[C@]4([2H])NCCO)no2)cc1C#N ; [2H]C(C)(C)Oc1ccc(-c2nc(-c3cccc4c3CC[C@@H]4NC([2H])([2H])C([2H])([2H])O)no2)cc1C#N] 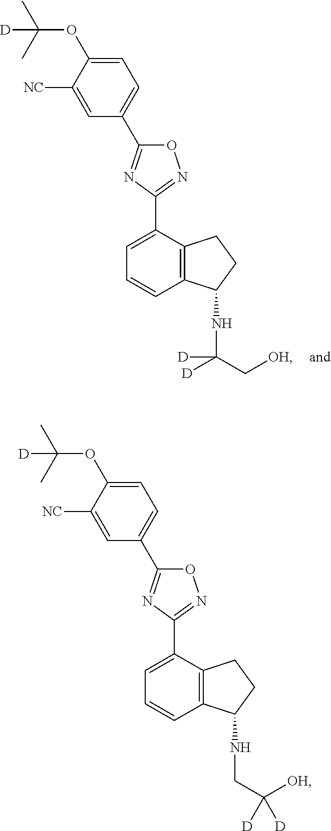 [SMILES: [2H]C([2H])(CO)N[C@H]1CCc2c(-c3noc(-c4ccc(OC([2H])(C)C)c(C#N)c4)n3)cccc21 ; [2H]C([2H])(O)CN[C@H]1CCc2c(-c3noc(-c4ccc(OC([2H])(C)C)c(C#N)c4)n3)cccc21]
or a salt thereof.

72. The compound as recited in claim 57 wherein said compound has a structural formula selected from the group consisting of 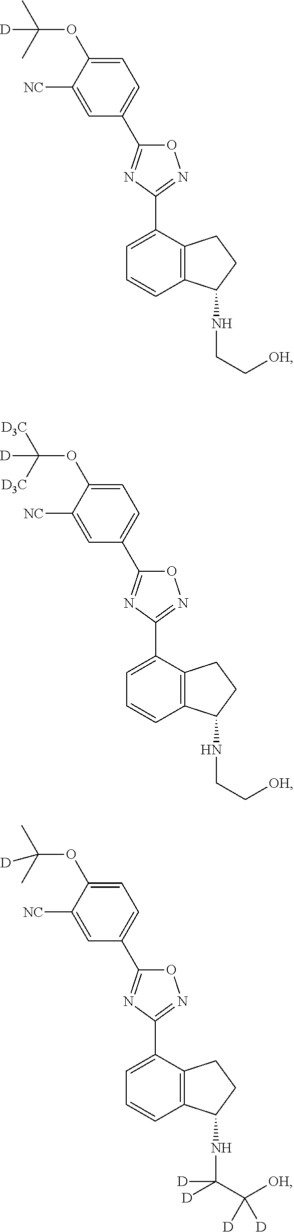 [SMILES: [2H]C(C)(C)Oc1ccc(-c2nc(-c3cccc4c3CC[C@@H]4NCCO)no2)cc1C#N ; [2H]C([2H])([2H])C([2H])(Oc1ccc(-c2nc(-c3cccc4c3CC[C@@H]4NCCO)no2)cc1C#N)C([2H])([2H])[2H] ; [2H]C(C)(C)Oc1ccc(-c2nc(-c3cccc4c3CC[C@@H]4NC([2H])([2H])C([2H])([2H])O)no2)cc1C#N] 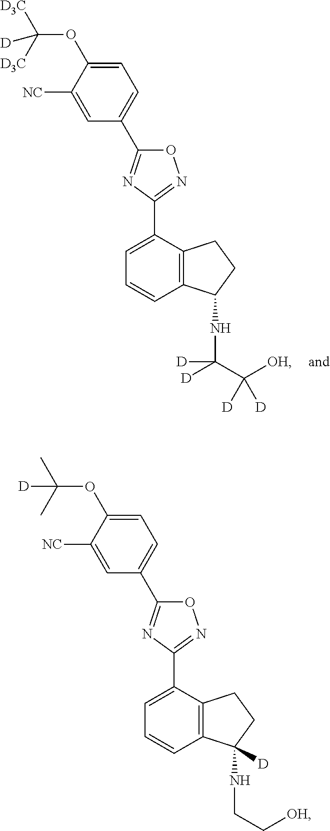 [SMILES: [2H]C([2H])([2H])C([2H])(Oc1ccc(-c2nc(-c3cccc4c3CC[C@@H]4NC([2H])([2H])C([2H])([2H])O)no2)cc1C#N)C([2H])([2H])[2H] ; [2H]C(C)(C)Oc1ccc(-c2nc(-c3cccc4c3CC[C@]4([2H])NCCO)no2)cc1C#N]
or a salt thereof.

73. The compound as recited in claim 72, wherein each position represented as D has deuterium enrichment of no less than about 10%.

74. The compound as recited in claim 73, wherein each position represented as D has deuterium enrichment of no less than about 10%.

75. A pharmaceutical composition comprising a compound as recited in claim 57 together with a pharmaceutically acceptable carrier.

76. A method of treatment or prevention of a S1P1 receptor-mediated disorder comprising the administration, to a patient in need thereof, of a therapeutically effective amount of a compound as recited in claim 57.
